(19) United States
(12) Patent Application Publication
(10) Pub. No.: US 2005/0089885 A1
(43) Pub. Date: Apr. 28, 2005

(54) IRF6 polymorphisms associated with cleft lip and/or palate

(75) Inventors: Brian C. Schutte, Iowa City, IA (US); Jeffrey C. Murray, Iowa City, IA (US); Shinji Kondo, Iowa City, IA (US); Michael J. Dixon, Manchester (GB)

Correspondence Address: MCKEE, VOORHEES & SEASE, P.L.C., 801 GRAND AVENUE, SUITE 3200, DES MOINES, IA, 50309-2721, US

(73) Assignee: University of Iowa Research Foundation, Iowa City, IA (US)

(21) Appl. No.: 10/840,139

(22) Filed: May 6, 2004

Related U.S. Application Data
(60) Provisional application No. 60/468,191, filed on May 6, 2003.

(51) Int. Cl. C12Q 1/68

(52) U.S. Cl. 435/6

(57) ABSTRACT

This invention relates to polymorphisms in the IRF6 gene which are associated with various diseases, such as Van der Woude syndrome, Poplitieal syndrome, isolated cleft lip and/or palate, and other orofusial clefting disorders. The invention also relates to the nucleotide sequences of this gene, isolated nucleic acids comprising these nucleotide sequences, and isolated polypeptides or peptides encoded thereby. The invention further relates to vectors and host cells comprising the disclosed nucleotide sequences, or fragments thereof, as well as antibodies that bind to the encoded polypeptides or peptides. In addition the invention relates to methods and compositions employing the disclosed nucleic acids, polypeptides or peptides, or antibodies for use in diagnostics and therapeutics for IRF6 associated disorders, e.g., Van der Woude syndrome, Poplitieal syndrome, isolated cleft lip and/or palate, and other orofusial clefting disorders.

### CROSS-REFERENCE TO RELATED APPLICATIONS

This application claims priority to Provisional Application 60/468,191, filed on May 6, 2003, herein incorporated by reference in its entirety.

### GRANT REFERENCE

Work for this invention was funded by grants from the National Institutes of Health (NIH) Grant Reference Nos.: NIH RO1 DE13513, RO1 DE08559, RO1 ES10876 and P60 DE13076. The United States government may have certain rights in this invention.

### BACKGROUND OF THE INVENTION

Interferon regulatory factor 6 (IRF6) belongs to a family of nine transcription factors that share a highly conserved helix-turn-helix DNA-binding domain and a less conserved protein-binding domain. Most IRFs regulate the expression of interferon-alpha and -beta after viral infection (Taniguchi et al., 2001), but the function of IRF6 is unknown. Van der woude syndrome (VWS) (OMIM 119300) is an autosomal dominant form of cleft lip and palate with lip pits (van der Woude, 1954), and is the most common syndromic form of cleft lip or palate. Popliteal pterygium syndrome (PPS) (OM 119500) is a disorder with a similar orofacial phenotype that also includes skin and genital anomalies (Gorlin et al., 1968). Phenotypic overlap (Bixler et al., 1973) and linkage data (Lees et al., 1999) suggest that these two disorders are allelic.

Cleft lip and/or palate (CL/P) is a common birth defect with prevalence varying according to geographic origin with Asian and Ameri-Indian populations having the highest rates and African-derived groups the lowest (Mossey et al., 2002). Isolated CL/P comprises 70% of all disorders with a cleft, with the remaining 30% being divided across several hundred Mendelian, chromosomal, teratogenic, and sporadic conditions that typically include other birth defects. Clefts of the lip with or without cleft palate and isolated cleft palate are developmentally and genetically distinct (Fraser et al., 1955), yet VWS is a single-gene disorder that encompasses both clefting phenotypes.

Early diagnosis of these diseases or predispositions to these diseases would be desirable. Therefore, it can be seen from the foregoing that a need exists for the identification of IRF6 gene types associated with Van der Woude and Popliteal pterygium syndromes, isolated cleft lip and/or palate (denoted herein as CLIP), or any other such cleft lip disorder.

An object of the present invention is to provide polymorphisms that are associated with IRF6 dysfunctions or dysregulation comprising, e.g., Van der Woude syndrome, Poplitieal pterygium syndrome, isolated cleft lip and/or palate (CLIP), or other such cleft lip disorders.

Another object of the invention is to provide novel alleles that are associated with IRF6 dysfunctions or dysregulation comprising, e.g., Van der Woude syndrome, Poplitieal pterygium syndrome, isolated cleft lip and/or palate (CLIP), or other such cleft lip disorders.

Yet another object of the invention is to provide methods for identifying such polymorphisms.

Another object of the invention is to provide methods of diagnosing a disease or disorder associated with IRF6 dysfunctions or dysregulation comprising, e.g., Van der Woude syndrome, Poplitieal pterygium syndrome, isolated cleft lip and/or palate (CL/P), or other such cleft lip disorders.

Additional objects and advantages of the invention will be set forth in part in the description that follows, and in part will be obvious from the description, or may be learned by the practice of the invention. The objects and advantages of the invention will be attained by means of the instrumentality's and combinations pointed out in the appended claims.

All publications cited herein are hereby incorporated by reference in their entirety.

### BRIEF SUMMARY OF THE INVENTION

As described herein, it has been discovered that polymorphisms in the nucleotide sequence coding for Interferon regulatory factor 6 (IRF6) are associated with Van der Woude and Popliteal pterygium syndromes. In particular multiple mutations in the IRF6 gene have been identified and found to be present in humans affected with one of these diseases. Regions of the IRF6 protein encoding sequence were identified having a propensity for mutation which was correlated with the diseases disclosed herein. For example, of the missense mutations, 35 of 37 localized to regions encoding either the winged helix DNA binding domain, (amino acids 13-113) or a protein-binding domain (amino acids 226-394) called SMIR (Smad-interferon regulatory factor-binding domain). VWS causing mutations were found evenly dispersed between the two domains. PPS were found primarily in the DNA-binding domain.

Every amino acid residue that was mutant in people with PPS directly contacted the DNA whereas only seven of the mutant residues in VWS contacted the DNA. Seven mutations associated with PPS involved the Arg84 residue. The Arg84 is comparable to the Arg82 residue of IRF1. It is one of four residues that make critical contacts with the core sequence, GAAA, and is essential for DNA binding (Escalante et al., 1998). The observed change of this residue to a cysteine or histidine caused a complete loss of that essential contact.

Also discovered was a common polymorphic variant, V274I in the protein-binding domain of the IRF6 gene, which affects gene function and contributes to CL/P.

Usually, the amino acid substitutions in the amino acid sequence of the protein encoded by the polynucleotide of the invention are due to one or more nucleotide substitutions. Preferably the nucleotide substitutions result in an amino acid substitution of Arg at position corresponding to position 84 of the IRF6 polypeptide (GenBank Accession No. NM_{—}006147) (GenBank Version No. NM_{—}006147.2). The mutations in the IRF6 gene detected in accordance with the present invention are listed in Table 1.

TABLE 1
IRF6 mutations

| | | | | |
|---|---|---|---|---|
| Family | Mutation | nt change | aa change | Exon |

| | | | | |
|---|---|---|---|---|
| VWS1 | frameshift | A-48T | 5′UTR to Met | 2 |
| VWS2 | frameshift | G3A | Met1lle | 3 |
| VWS3 | missense | C5T | Ala2Val | 3 |
| VWS4 | frameshift | 17ins(C) | Arg6fs | 3 |
| VWS35 | frameshift | 49del | Gln17fs | 3 |
| | | (CAGGTGGATAGTG | | |
| | | GCC) | | |
| VWS5 | missense | G52A | Val18Met | 3 |
| VWS36 | missense | T53C | Val18Ala | 3 |
| VWS37 | nonsense | C69A | Tyr23X | 3 |
| VWS6 | missense | C115G | Pro39Ala | 3 |
| PPS1 | missense | T178G | Trp60Gly | 4 |
| VWS7 | missense | C182G | Ala61Gly | 4 |
| PPS2 | missense | A197C | Lys66Thr | 4 |
| VWS8 | nonsense | C202T | Gln68X | 4 |
| VWS9 | nonsense | C202T | Gln68X | 4 |
| VWS10 | missense | G208C | Gly70Arg | 4 |
| VWS11 | missense | G208C | Gly70Arg | 4 |
| VWS45 | missense | C226T | Pro76Ser | 4 |
| PPS13 | missense | C244A | Gln82Lys | 4 |
| PPS3 | missense | C250T | Arg84Cys | 4 |
| PPS4 | missense | C250T | Arg84Cys | 4 |
| PPS5 | missense | C250T | Arg84Cys | 4 |
| PPS6 | missense | C250T | Arg84Cys | 4 |
| PPS7 | missense | C250T | Arg84Cys | 4 |
| PPS8 | missense | G251A | Arg84His | 4 |
| PPS9 | missense | G251A | Arg84His | 4 |
| VWS12 | missense | A262C | Asn88His | 4 |
| PPS10 | missense | A265G | Lys89Glu | 4 |
| VWS13 | missense | A268G | Ser90Gly | 4 |
| VWS14 | nonsense | G274T | Glu92X | 4 |
| VWS41 | nonsense | G274T | Glu92X | 4 |
| VWS15 | missense | G292C | Asp98His | 4 |
| VWS16 | nonsense | C352T | Gln118X | 4 |
| VWS17 | frameshift | 466ins(C) | His156fs | 5 |
| VWS18 | nonsense | C558A | Cys186X | 6 |
| VWS19 | nonsense | G576A | Trp192X | 6 |
| VWS20 | frameshift | 634in(CCAC) | Ser212fs | 6 |
| VWS21 | frameshift | 657del | Ser219fs | 6 |
| | | (CTCTCTCCC)ins | | |
| | | (TA) | | |
| VWS42 | frameshift | 744del(CTGCC) | Gly248fs | 7 |
| VWS22 | missense | G749A | Arg250Gln | 7 |
| VWS43 | nonsense | T759A | Tyr253X | 7 |
| VWS44 | frameshift | 795del(C) | Leu265fs | 7 |
| VWS23 | missense | A818G | Gln273Arg | 7 |
| VWS24 | frameshift | 842del(A) | His281fs | 7 |
| VWS25 | deletion | 870del | FTSKLLD290L | 7 |
| | | (CACTAGCAAGCT | | |
| | | GCTGGAC)lns(A) | | |
| | | (SEQ ID NO:3) | | |
| VWS46 | missense | T881C | Leu294Pro | 7 |
| VWS26 | missense | G889A | Val297Ile | 7 |
| VWS38 | missense | A958G | Lys320Glu | 7 |
| VWS39 | missense | A958G | Lys320Glu | 7 |
| VWS27 | missense | G961A | Val321Met | 7 |
| VWS40 | missense | G974A | Gly325Glu | 7 |
| VWS28 | missense | T1034C | Leu345Pro | 7 |
| VWS29 | missense | G1040T | Cys347Phe | 7 |
| VWS30 | missense | T1106C | Phe369Ser | 8 |
| VWS31 | missense | C1122G | Cys374Trp | 8 |
| VWS32 | missense | A1162G | Lys388Glu | 8 |
| PPS11 | nonsense | C1177T | Gln393X | 8 |
| VWS33 | nonsense | C1234T | Arg412X | 9 |
| PPS12 | missense | G1288A | Asp430Asn | 9 |
| VWS34 | frameshift | 1381ins(C) | Pro461fs | 9 |
| CL/P | | | Val274Ile | 7 |

Nucleotide position is relative to start codon. Mutations in the DNA-binding and SMIR/IAD domains are located in the top and bottom box, respectively.

Mutations or polymorphisms associated with VWS, PPS, or CL/P in IRF6 were found in exons 2-9 (FIG. 3). More specifically, as shown in Table 1, mutations associated with VWS were identified in exons 2, 3, 4, 5, 6, 7, 8 and 9. Mutations associated with PPS were identified in exons 4, 8 and 9 and a mutation associated with CL/P was identified in exon 7 of IRF6.

The novel polymorphic markers found associated with VWS are as follows:

In exon 2 is: a nucleotide change of A-48T which corresponds to an amino acid change in the 5′UTR to methionine (frameshift).

In exon 3: a nucleotide change of G3A which corresponds to amino acid change Met1Ile (frameshift); a nucleotide change of C5T which corresponds to amino acid change Ala2Val (missense); a nucleotide change of 17ins(C) which corresponds to amino acid change Arg6fs (frameshift); a nucleotide change of 49 del (CAGGTGGATAGTGGCC) which corresponds to amino acid change Gln17fs (frameshift); a nucleotide change of G52A which corresponds to amino acid change Val18Met (missense); a nucleotide change of T53C which corresponds to amino acid change Val18Ala (missense); a nucleotide change of C69A which corresponds to amino acid change Tyr23X (nonsense); a nucleotide change of C115G which corresponds to amino acid change Pro39Ala (missense).

In exon 4: a nucleotide change of C182G which corresponds to amino acid change Ala61Gly (missense); a nucleotide change of C202T which corresponds to amino acid change Gln68X (nonsense); a nucleotide change of G208C which corresponds to amino acid change Gly70Arg (missense); a nucleotide change of C226T which corresponds to amino acid change Pro76Ser (missense); a nucleotide change of A262C which corresponds to amino acid change Asn88His (missense); a nucleotide change of A268G which corresponds to amino acid change Ser90Gly (missense); a nucleotide change of G274T which corresponds to amino acid change Glu92X (nonsense); a nucleotide change of G292C which corresponds to amino acid change Asp98His (missense); a nucleotide change of C352T which corresponds to amino acid change Gln118X (nonsense).

In exon 5: a nucleotide change of 466ins(C) which corresponds to amino acid change His156fs (frameshift).

In exon 6: a nucleotide change of C558A which corresponds to amino acid change Cys186X (nonsense); a nucleotide change of G576A which corresponds to amino acid change Trp192X (nonsense); a nucleotide change of 634in(CCAC) which corresponds to amino acid change Ser212fs (frameshift); a nucleotide change of 657del(CTCTCTCCC)ins(TA) which corresponds to amino acid change Ser219fs (frameshift).

In exon 7: a nucleotide change of 744del (CTGCC) which corresponds to amino acid change Gly248fs (frameshift); a nucleotide change of G749A which corresponds to amino acid change Arg250Gln (missense); a nucleotide change of T759A which corresponds to amino acid changeTyr253X (nonsense); a nucleotide change of 795del(C) which corresponds to amino acid change Leu265fs (frameshift); a nucleotide change of A818G which corresponds to amino acid change Gln273Arg (missense); a nucleotide change of 842del(A) which corresponds to amino acid change His281fs (frameshift); a nucleotide change of 870del (CACTAGCAAGCTGCTGGAC)ins(A) which corresponds to amino acid change FTSKLLD290L (deletion); a nucleotide change of T881C which corresponds to amino acid change Leu294Pro (missense); a nucleotide change of G889A which corresponds to amino acid change Val297Ile (missense); a nucleotide change of A958G which corresponds to amino acid change Lys320Glu (missense); a nucleotide change of G961A which corresponds to amino acid change Val321Met (missense); a nucleotide change of G974A which corresponds to amino acid change Gly325Glu (missense); a nucleotide change of T1034C which corresponds to amino acid change Leu345Pro (missense); a nucleotide change of G1040T which corresponds to amino acid change Cys347Phe (missense).

In exon 8: a nucleotide change of T1106C which corresponds to amino acid change Phe369Ser (missense); a nucleotide change of C1122G which corresponds to amino acid change Cys374Trp (missense); a nucleotide change of A1162G which corresponds to amino acid change Lys388Glu (missense).

In exon 9: a nucleotide change of C1234T which corresponds to amino acid change Arg412X (nonsense); a nucleotide change of 1381ins(C) which corresponds to amino acid change Pro461fs (frameshift).

The novel polymorphic markers found associated with PPS are as follows:

In exon 4 are: a nucleotide change of T178G which corresponds to amino acid change Trp60Gly (missense); a nucleotide change of A197C which corresponds to amino acid change Lys 66Thr (missense); a nucleotide change of C244A which corresponds to amino acid change Gln82Lys (missense); a nucleotide change of C250T which corresponds to amino acid change Arg84Cys (missense); a nucleotide change of G251A which corresponds to amino acid change Arg84His (missense); a nucleotide change of A265G which corresponds to amino acid change Lys89Glu (missense).

In exon 8: a nucleotide change of C1177T which corresponds to amino acid change Gln393X (nonsense).

In exon 9: G1288A which corresponds to amino acid change Asp430Asn (missense).

The novel polymorphic marker found associated with CL/P in exon 7 is: an amino acid change Val274Ile.

The variant polynucleotides and methods disclosed herein may be used for evaluating the phenotypic spectrum as well as the overlapping clinical characteristics of diseases or conditions related to dysfunctions or dysregulations related to development of cleft lip and/or palate, which are also referred to herein also as IRF6-related disorders.

The polymorphic polynucleotides referred to in the present invention which contain at least two of the mutations in Table 1 may be used to determine haplotypes. Haplotypes can be used to identify chromosomal abnormalities linked to these disorders disclosed herein. Additionally, this allows the study of synergistic effects of the mutations in the IRF6 gene and/or a polypeptide encoded by the polynucleotide on the pharmacological profile of drugs in patients who bear such mutant forms of the gene or similar mutant forms that can be mimicked by the above described proteins.

Accordingly, this invention pertains to an isolated nucleic acid molecule comprising the IRF6 gene of SEQ ID NO: 1 having at least one altered nucleotide and to gene products encoded thereby (referred to herein as a “variant IRF6 gene or nucleic acid” or “variant IRF6 gene product” or “polymorphic IRF6 gene or nucleic acid” or “polymorphic IRF6 gene product”) as set forth in Table 1.

A number of polymorphisms reported in Table 1 have been observed in the IRF6 gene. Thus, in preferred embodiments, the isolated nucleic acid molecule of the invention can have one or a combination of these polymorphisms. These polymorphisms may be part of a group of other polymorphisms in the IRF6 gene which contributes to the absence, presence, or prevalence of VWS, PPS, or CL/P. In a particularly preferred embodiment, the nucleic acid molecule will comprise at least one polymorphism at amino acid residue 84 of a polymorphic gene product.

The invention further provides a method of diagnosing an IRF6-related disorder in a subject. Diagnosing assays can be designed to asses whether a person has an IRF6 related disorder is susceptible thereto. Such methods comprise obtaining a biological sample from said subject, wherein said sample comprises the IRF6 nucleic acid; and detecting a polymorphism in said IRF6 nucleic acid, wherein the presence of a polymorphism is indicative of said subject having an IRF6-related disorder. The invention additionally provides a method of diagnosing an IRF6-related disorder in a subject, the method comprising obtaining a biological sample from said subject; analyzing the IRF6 nucleic acid in said sample obtained from said subject; and determining the presence of at least one mutation as set forth in Table 1 of IRF6 in said subject, wherein the presence of said mutation is indicative of said subject having an IRF6-related disorder.

The invention further provides an isolated nucleic acid molecule comprising: (a) a nucleic acid molecule comprising an IRF6 nucleic acid having the nucleotide sequence of SEQ ID NO:1 and comprising at least one polymorphism as shown in Table 1; (b) a nucleic acid molecule comprising an IRF6 gene, the nucleotide sequence of SEQ ID NO:1 encoding a polypeptide comprising an amino acid sequence as depicted in SEQ ID NO:2 and comprises at least one polymorphism associated as shown in Table 1. Variant IRF6 polynucleotides are useful as genetic markers for diagnosing a predisposition to VWS, PPS, CL/P, or other such cleft lip related disorders and for diagnosing VWS, PPS, or CL/P disorders. Additionally, variant IRF6 polynucleotides, as well as their gene products, as genetic or biochemical markers (e.g., blood or tissues) that could detect propensity to VWS, PPS, CL/P, or other such cleft lip related disorders.

The invention also relates to a vector comprising an isolated nucleic acid molecule of the invention operatively linked to a regulatory sequence, as well as to a recombinant host cell comprising the vector. Vectors of the invention can be designed for expression of a polypeptide of the invention in prokaryotic or eukaryotic cells, e.g., a bacterial, insect, fungal, plant, animal, mammalian or, preferably, human cell for drug screening protocols and other models of cleft lip causing proteins.

The invention further provides an isolated IRF6 polypeptides encoded by isolated nucleic acid molecules of the invention (e.g., a variant IRF6 polypeptide). Polypeptides of the invention may be used, for example, to identify agents which bind to the protein or interact with the variant polypeptide in drug screening assays, for example. Further, the variant polypeptides of the invention may be used to raise antibodies in a suitable host. The variant polypeptides disclosed herein are also useful as targets for therapeutic intervention, which encompasses treatment, prognosis, or amelioration of one or more mutant forms born by individual at risk or having the disorders disclosed herein. In a particular embodiment, the polypeptide comprises the amino acid sequence of SEQ ID NO:2 and comprising at least one polymorphism as shown in Table 1. Isolated polymorphic IRF6 polypeptides of the invention are useful for the production of antibodies, where short fragments provide for antibodies specific for the particular polypeptide and larger fragments or the entire protein allow for the production of antibodies over the entire surface of the polypeptide. In some embodiments, it is preferable that the polymorphism results in an amino acid substitution of Arg at a position corresponding to position 84 of the IRF6 polypeptide (GenBank Accession No. NP_{—}006138.1).

The invention also relates to antibodies, or an antibody fragment thereof, that are specific for the polymorphic polypeptide of the invention. The antibodies of the invention are useful in a variety of diagnostic assays as described herein. For example, an antibody of the invention can be used to detect polymorphic IRF6 polypeptides having at least one of the mutations in it sequence as shown in Table 1.

The invention additionally relates to an assay for identifying agents, e.g., drugs or prodrugs which alter (e.g., enhance or inhibit) the activity of one or more variant IRF6 polypeptides if the invention. For example, a cell, cellular fraction, or solution containing an IRF6 polypeptide or a fragment or derivative thereof, can be contacted with an agent to be tested, and the level of the variant IRF6 polypeptide activity can be assessed in the presence and absence of the agent and the levels compared. If the level of activity in the presence of the agent differs by an amount that is statistically significant from the activity in the absence of the same agent, the agent is an agent that alters the expression or activity of a variant IRF6 polypeptide of the invention. The activity or expression of more than one variant IRF6 polypeptides can be assessed concurrently (e.g., the cell, cellular fraction, or solution can contain more than one type of IRF6 polypeptide, such as different splicing variants, and the levels of the different polypeptides or splicing variants can be assessed). Agents or compounds that enhance or inhibit IRF6 polypeptide expression or activity are also included in the present invention.

The invention also relates to pharmaceutical compositions to treat or ameliorate the symptoms of cleft lip and/or palate and other IRF6-related disorders comprising an IRF6 wild-type protein or nucleic acid encoding sequence and a pharmaceutical carrier.

### BRIEF DESCRIPTION OF THE DRAWINGS

FIG. 1 provides a nucleotide sequence of a human IRF6 gene (SEQ ID NO:1).

FIG. 2 provides a polypeptide sequence of a human IRF6 amino acid sequence (SEQ ID NO:2).

FIG. 3 provides the structure of the IRF6 gene. Exons (rectangles) are drawn to scale except exon 9, which is longer than shown. The brackets connecting the exons represent spliced introns, and the break between exons 9 and 10 represents an unspliced intron of 1,621 nucleotides (nt) that is present in the most common 4.4-kb IRF6 transcript. The predicted IRF6 protein contains a winged-helix DNA binding domain and a SMIR/IAD protein-binding domain. The DNA-binding domain includes a pentatryptophan (w) motif. The arrowheads indicate the relative position of protein-truncation (above exons) and missense mutations (below exons) that causes VWS or PPS or that are polymorphisms. The arrow above exon 4 represents the Glu92 nonsense mutation identified in the affected twin of family VWS14. The amino acid change for each missense mutation is shown and an asterisk indicates mutations affecting residues that contact the DNA.

FIG. 4 is a schematic of the IRF6 gene locus with thin black arrows and numbering indicating the locations of the SNPs genotyped in either the Filipino and/or Danish/Iowa triads, except for the V2741 variant which is indicated in bold black.

FIG. 5 provides a summary of the Odds Ratios (ORs) for overtransmission of the V allele of V274I in proband nuclear families where the proband has cleft lip with or without cleft palate by population and subgroups. The “Combined” result includes all populations from a particular geographic region (except for ECLAMC). The ORs are also shown for haplotypes defined in FIG. 8 in three populations. The circles show the point estimate and the vertical bars indicate the 95% confidence interval.

FIG. 6 provides the overtransmission for each allele above 50% is shown for each of the 36 SNPs studied in the Filipinos. Variants where the overtransmission is significant with P<0.01 are indicated in black, all others hashed.

FIG. 7 provides an individual P-values for each of the 36 SNPs typed in the 296 triads from the Philippines are plotted according to their locations on the chromosome. The regions of mouse homology that were sequenced are shown in relation to these markers.

FIG. 8 provides haplotypes with a frequency of >1% in the Filipino triads (top) and Danish/Iowa combined triads (bottom). For the Filipinos, the haplotype analysis only included those SNPs with a P-value of <0.009 for FEAT tests in that population. Haplotypes are described by the black representing the common allele at each SNP and white representing the rare allele at each SNP. For the significantly associated haplotypes, P-values are given and whether the association is positive or negative with the haplotype shown.

### DETAILED DESCRIPTION OF THE PREFERRED EMBODIMENT

As described herein, mutations in the IRF6 gene result in Van der Woude syndrome (VWS) and Popliteal pterygium syndrome (PPS). Additionally, it has been discovered that a mutation in the IRF6 gene associated with isolated cleft lip and/or palate (CL/P). VWS is an autosomal dominant form of cleft lip and palate associated with lip pits, and is the most common syndromic form of cleft lip or palate. PPS is a disorder with a similar orofacial phenotype that also includes skin and genital anomalies. To associate the expression of IRF6 with the phenotypes of VWS and PPS, Applicants conducted expression analyses in mice. Studies showed high levels of IRF6 mRNA along the medial edge of the fusing palate, tooth buds, hair follicles, genitalia, and skin. Their observations demonstrated that haploinsufficiency of IRF6 disrupts orofacial development and were consistent with dominant-negative mutations disturbing development of the skin and genitalia. Additionally, Applicants identified the IRF6 gene within the VWS critical region at 1q32-q41. Further, Applicants have identified a common polymorphic variant, V247I (as listed in Table 1), in the protein binding domain of IRF6.

All nucleotide positions in Table 1 are relative to start codon.

Definitions

As used herein the term “IRF6 gene” is intended to generically refer to both the wild-type and variant forms of the sequences, unless specifically denoted otherwise. As it is commonly used in the art, the term “gene” is intended to refer to the genomic regions encompassing 5′ untranslated regions(s) (UTR), exons, introns, and 3′ UTR. Individual segments may be specifically referred to, e.g., exon 2 etc. Combinations of such segments that provide for a complete IRF6 protein may be referred to generically as a protein coding sequence. The nucleotide sequences of IRF6 mRNA are publicly available through Genbank: Accession No. NM_{—}006147 (human IRF6 mRNA).

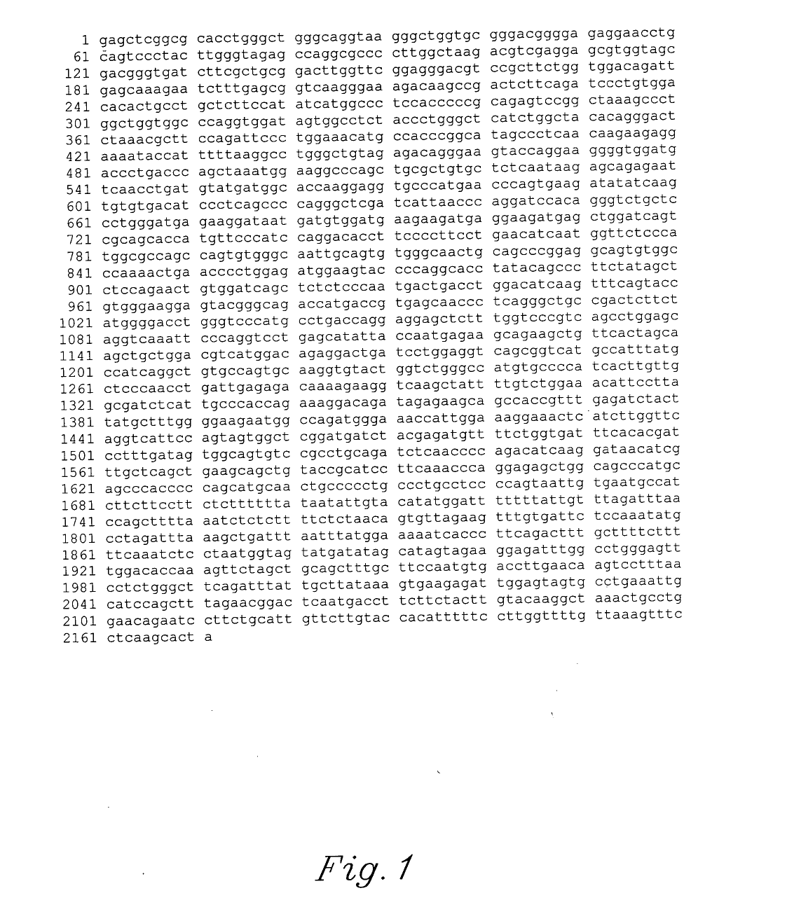

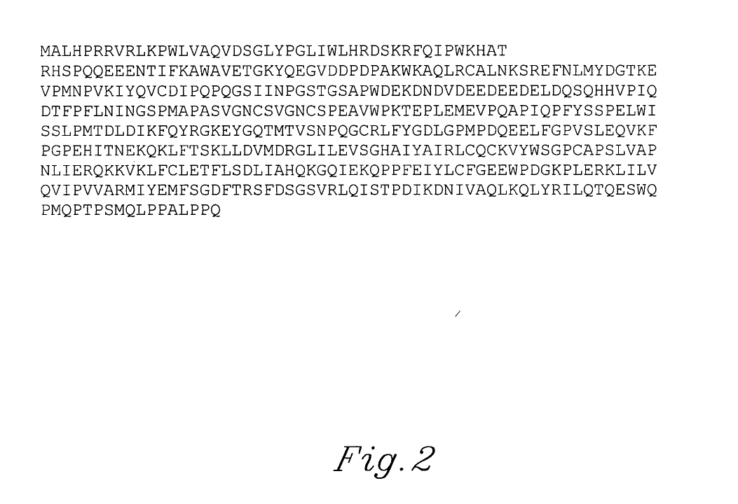

The term “polymorphism”, as used herein refers to a difference in a nucleotide or amino acid sequence of a given region as compared to a nucleotide or amino acid sequence in a homologous region of another individual, in particular, a difference in the nucleotide or amino acid sequence of a given region which differs between individuals of the same species. A polymorphism is generally defined in relation to a reference sequence. As disclosed herein, FIGS. 1 and 2 are reference sequences. Polymorphisms include single nucleotide single nucleotide differences, differences in sequence of more than one nucleotide, and single or multiple nucleotide insertions, inversions, deletions; as well as single amino acid difference, differences in sequence of more than one amino acid, and single or multiple amino acid insertions, inversions, and deletions.

As used herein, the term “polymorphic IRF6 nucleic acid molecule” or “variant IRF6 nucleic acid molecule” refers to a polynucleotide derived from an IRF6 gene, which comprises one or more polymorphisms as shown in Table 1, when compared to a reference IRF6 polynucleotide sequence.

The terms “polynucleotide” and “nucleic acid molecule” are used interchangeably herein to refer to polymeric forms of nucleotides of any length. The polynucleotides may contain deoxyribonucleotides, ribonucleotides, and/or their analogs. Nucleotides may have any three-dimensional structure, and may perform any function, known or unknown. The term “polynucleotide” includes single-, double-stranded and triple helical molecules. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as oligomers or oligos and may be isolated from genes, or chemically synthesized by methods known in the art.

The following are non-limiting embodiments of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A nucleic acid molecule may also comprise modified nucleic acid molecules, such as methylated nucleic acid molecules and nucleic acid molecule analogs. Analogs of purines and pyrimidines are known in the art. Modifications in the native structure, including alterations in the backbone, sugars or heterocyclic bases, have been shown to increase intracellular stability and binding affinity. Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH2-5′-O-phosphonate and 3′-NH-5′-O-phosphoroamidate. Peptide nucleic acids replace the entire ribose phosphodiester backbone with a peptide linkage.

Sugar modifications are also used to enhance stability and affinity. The alpha-anomer of deoxyribose may be used, where the base is inverted with respect to the natural beta-anomer. The 2′-OH of the ribose sugar may be altered to form 2′-O-methyl or 2′-O-allyl sugars, which provides resistance to degradation without comprising affinity.

Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

The terms “polypeptide” and “protein”, used interchangeably herein, refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

The term “hybridizing” as used herein refers to polynucleotides which are capable of hybridizing to the polynucleotides of the invention or parts thereof which are associated with an IRF6 dysfunction or dysregulation. Thus, said hybridizing polynucleotides are also associated with said dysfunctions and dysregulations.

The term “corresponding” as used herein means that a position is not only determined by the number of the preceding nucleotides and amino acids, respectively. The position of a given nucleotide or amino acid in accordance with the present invention which may be deleted, substituted or comprise one or more additional nucleotide(s) may vary due to deletions or additional nucleotides or amino acids elsewhere in the gene or the polypeptide. Thus, under a “corresponding position” in accordance with the present invention it is to be understood that nucleotides or amino acids may differ in the indicated number but may still have similar neighboring nucleotides or amino acids. Said nucleotides or amino acids which may be exchanged, deleted or comprise additional nucleotides or amino acids are also comprised by the term “corresponding position”. Said nucleotides or amino acids may for instance together with their neighbors form sequences which may be involved in the regulation of gene expression, stability of the corresponding RNA or RNA editing, as well as encode functional domains or motifs of the protein of the invention.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polynucleotides. The term “biological sample” encompasses a clinical sample, and also includes cells in culture, cell supernatants, cell lysates, serum, plasma, biological fluid, and tissue samples.

The term “isolated fractions thereof” refers to fractions of eukaryotic or prokaryotic cells or tissues which are capable of transcribing or transcribing and translating RNA from the vector of the invention. Said fractions comprise proteins which are required for transcription of RNA or transcription of RNA and translation of said RNA into a polypeptide. The isolated fractions may be, e.g., nuclear and cytoplasmic fractions of eukaryotic cells such as of reticulocytes.

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” and “patient,” used interchangeably herein, refer to a mammal, including, but not limited to, murines, simians, humans, mammalian farm animals, mammalian sport animals, and mammalian pets.

A gene sequence is “wild-type” if such sequence is usually found in individuals unaffected by the disease or condition of interest. However, environmental factors and other genes can also play an important role in the ultimate determination of the disease. In the context of complex diseases involving multiple genes (“oligogenic disease”), the “wild type” or normal sequence can also be associated with a measurable risk or susceptibility, receiving its reference status based on its frequency in the general population.

A gene sequence is a “mutant” sequence if it differs from the wild-type sequence. In some cases, the individual carrying such gene has increased susceptibility toward the disease or condition of interest. In other cases, the “mutant” sequence might also refer to a sequence that decreases the susceptibilty toward a disease or condition of interest, and thus acting in a protective manner. Also a gene is a “mutant” gene if too much (“overexpressed”) or too little (“underexpressed”) of such gene is expressed in the tissues in which such gene is normally expressed, thereby causing the disease or condition of interest.

Variant IRF6 polynucleotides of the invention are useful genetic markers for diagnosing a predisposition to an IRF6-related disorder such as VWS, PPS or CL/P, or such cleft lip related disorders and for diagnosing VWS, PPS or CL/P, or such cleft lip related disorders. Moreover, the variant IRF6 polynucleotides of the invention can be used in diagnostic (i.e., detection) and screening methods. Variant IRF6 polynucleotides disclosed herein, as well as their variant gene products, are also useful as genetic or biochemical markers (e.g., in blood or tissues) that could detect propensity to an IRF6-related disorder, and/or to monitor the efficacy of various therapies and preventative interventions.

Accordingly, the invention pertains to an isolated nucleic acid molecule comprising the human IRF6 gene having at least one nucleotide alteration and correlated with the phenotypes of VWS, PPS or CL/P.

The term, “variant IRF6 ”, as used herein, refers to an isolated nucleic acid molecule in chromosome 1 having at least one altered nucleotide that is associated with a susceptibility to a number of VWS, PPS, or CL/P phenotypes, and also to a portion or fragment of the isolated nucleic acid molecule containing the alteration (e.g., cDNA or the gene) and encoding a variant IRF6 polypeptide (e.g., the polypeptide having SEQ ID NO: 2). In a preferred embodiment, the isolated nucleic acid molecules comprise a polymorphism selected from the group consisting of any one or a combination of those shown in Table 1. In certain embodiments for therapeutic purposes for example, the nucleic acid comprises the sequence of SEQ ID NO: 1 which represents the human IRF6 gene of a healthy subject.

The isolated nucleic acid molecule of the present invention can be RNA, for example, mRNA, or DNA, such as cDNA and genomic DNA. DNA molecules can be double-stranded or single-stranded; single stranded RNA or DNA can be either the coding, or sense, strand or the non-coding, or antisense strand. The nucleic acid molecule can include all or a portion of the coding sequence of the gene and can further comprise additional non-coding sequences such as introns and non-coding 3′ and 5′ sequences (including regulatory sequences, for example). Additionally, the nucleic acid molecule can be synthetically produced DNA or RNA or a recombinantly produced chimeric nucleic acid molecule comprising any of the nucleic acid sequences either alone or in combination having at least one polymorphism as set forth in Table 1. The nucleic acid molecule can be fused to a marker sequence, for example, a sequence that encodes a polypeptide to assist in isolation or purification of the polypeptide. Such sequences are well known in the art.

Isolated polynucleotides of the invention having at least two of the polymorphisms shown in Table 1 may be used to determine a haplotype. Haplotypes can be used to identify chromosomal abnormalities linked to the disorders disclosed herein. Additionally, this allows the study of synergistic effects of the mutations in the IRF6 gene and/or a polypeptide encoded by said polynucleotide on the pharmacological profile of drugs in patients who bear such mutant forms of the gene or similar mutant forms that can be mimicked by the variant polypeptides described herein.

An “isolated” nucleic acid molecule, as used herein, is one that is separated from nucleic acids which normally flank the gene or nucleotide sequence (as in genomic sequences) and/or has been completely or partially purified from other transcribed sequences (e.g., as in an RNA library). For example, an isolated nucleic acid of the invention may be substantially isolated with respect to the complex cellular milieu in which it naturally occurs, or culture medium when produced by recombinant techniques, or chemical precursors or other chemicals when chemically synthesized. In some instances, the isolated material will form part of a composition (for example, blood or a crude extract containing other substances), buffer system or reagent mix. In other circumstances, the material may be purified to essential homogeneity, for example as determined by PAGE or column chromatography such as HPLC. Preferably, an isolated nucleic acid molecule comprises at least about 50, 80 or 90% (on a molar basis) of all macromolecular species present. With regard to genomic DNA, the term “isolated” also can refer to nucleic acid molecules which are separated from the chromosome with which the genomic DNA is naturally associated. For example, the isolated nucleic acid molecule can contain less than about 5 kb, 4 kb, 3 kb, 2 kb, 1 kb, 0.5 kb or 0.1 kb of nucleotides which flank the nucleic acid molecule in the genomic DNA of the cell from which the nucleic acid molecule is derived.

The nucleic acid molecule can be fused to other coding or regulatory sequences and still be considered isolated. Thus, recombinant DNA contained in a vector is included in the definition of “isolated” as used herein. Also, isolated nucleic acid molecules include recombinant DNA molecules in heterologous host cells, as well as partially or substantially purified DNA molecules in solution. “Isolated” nucleic acid molecules also encompass in vivo and in vitro RNA transcripts of the DNA molecules of the present invention. Also, isolated nucleotide sequences include recombinant DNA molecules in heterologous organisms, as well as partially or substantially purified DNA molecules in solution. In vivo and in vitro RNA transcripts of the DNA molecules of the present invention are also encompassed by “isolated” nucleotide sequences. Such isolated nucleotide sequences are useful in the manufacture of the encoded polypeptide, as probes for isolating homologous sequences (e.g., from other mammalian species), for gene mapping (e.g., by in situ hybridization with chromosomes), or for detecting expression of the gene in tissue (e.g., human tissue), such as by Northern blot analysis.

The invention also pertains to nucleic acid molecules which hybridize under high stringency hybridization conditions, such as for selective hybridization, to a nucleotide sequence described herein (e.g., nucleic acid molecules which specifically hybridize to a nucleotide sequence encoding polypeptides described herein, and, optionally, have an activity of the polypeptide). In one embodiment, the invention includes variants described herein which hybridize under high stringency hybridization conditions (e.g., those which do not cross hybridize to unrelated polynucleotides such as polynucleotides encoding a polypeptide different from the IRF6 polypeptides of the invention) to a nucleotide sequence comprising a nucleotide sequence selected from SEQ ID NO: 1 comprising at least one polymorphism as shown in Table 1 or the complement thereof, or a nucleotide sequence encoding an amino acid sequence of SEQ ID NO: 2 comprising at least one polymorphism as shown in Table 1. In a preferred embodiment, the variant which hybridizes under high stringency hybridizations has an activity of IRF6.

Such nucleic acid molecules can be detected and/or isolated by specific hybridization (e.g., under high stringency conditions). “Specific hybridization,” as used herein, refers to the ability of a first nucleic acid to hybridize to a second nucleic acid in a manner such that the first nucleic acid does not hybridize to any nucleic acid other than to the second nucleic acid (e.g., when the first nucleic acid has a higher similarity to the second nucleic acid than to any other nucleic acid in a sample wherein the hybridization is to be performed). “Stringency conditions” for hybridization is a term of art which refers to the incubation and wash conditions, e.g., conditions of temperature and buffer concentration, which permit hybridization of a particular nucleic acid to a second nucleic acid; the first nucleic acid may be perfectly (i.e., 100%) complementary to the second, or the first and second may share some degree of complementarity which is less than perfect (e.g., 70%, 75%, 85%, 95%). For example, certain high stringency conditions can be used which distinguish perfectly complementary nucleic acids from those of less complementarity. “High stringency conditions”, “moderate stringency conditions” and “low stringency conditions” for nucleic acid hybridizations are explained on pages 2.10.1-2.10.16 and pages 6.3.1-6.3.6 in Current Protocols in Molecular Biology (Ausubel, F. M. et al., “Current Protocols in Molecular Biology”, John Wiley & Sons, (1998), the entire teachings of which are incorporated by reference herein). The exact conditions which determine the stringency of hybridization depend not only on ionic strength (e.g., 0.2×SSC, 0.1×SSC), temperature (e.g., room temperature, 42° C., 68° C.) and the concentration of destabilizing agents such as formamide or denaturing agents such as SDS, but also on factors such as the length of the nucleic acid sequence, base composition, percent mismatch between hybridizing sequences and the frequency of occurrence of subsets of that sequence within other non-identical sequences. Preferably, stringent hybridization conditions refer to an overnight incubation at 42 C in a solution comprising 50% formamide hybridization solution, followed by at least two washing steps at 60 C in 0.2×SSC with 1% SDS for at least 15 minutes.

Thus, equivalent conditions can be determined by varying one or more of these parameters while maintaining a similar degree of identity or similarity between the two nucleic acid molecules. Typically, conditions are used such that sequences at least about 60%, at least about 70%, at least about 80%, at least about 90% or at least about 95% or more identical to each other remain hybridized to one another. By varying hybridization conditions from a level of stringency at which no hybridization occurs to a level at which hybridization is first observed, conditions which will allow a given sequence to hybridize (e.g., selectively) with the most similar sequences in the sample can be determined.

Exemplary conditions are also described in Krause, M. H. and S. A. Aaronson, Methods in Enzymology, 200:546-556 (1991). Also, in, Ausubel, et al., “Current Protocols in Molecular Biology”, John Wiley & Sons, (1998), which describes the determination of washing conditions for moderate or low stringency conditions. Washing is the step in which conditions are usually set so as to determine a minimum level of complementarity of the hybrids. Generally, starting from the lowest temperature at which only homologous hybridization occurs, each degree C. by which the final wash temperature is reduced (holding SSC concentration constant) allows an increase by 1% in the maximum extent of mismatching among the sequences that hybridize. Generally, doubling the concentration of SSC results in an increase in Tₘ of about 17° C. Using these guidelines, the washing temperature can be determined empirically for high, moderate or low stringency, depending on the level of mismatch sought.

For example, a low stringency wash can comprise washing in a solution containing 0.2×SSC/0.1% SDS for 10 min at room temperature; a moderate stringency wash can comprise washing in a prewarmed solution (42° C.) solution containing 0.2×SSC/0.1% SDS for 15 min at 42° C.; and a high stringency wash can comprise washing in prewarmed (68° C.) solution containing 0.1×SSC/0.1% SDS for 15 min at 68° C. Furthermore, washes can be performed repeatedly or sequentially to obtain a desired result as known in the art. Equivalent conditions can be determined by varying one or more of the parameters given as an example, as known in the art, while maintaining a similar degree of identity or similarity between the target nucleic acid molecule and the primer or probe used.

The percent identity of two nucleotide or amino acid sequences can be determined by aligning the sequences for optimal comparison purposes (e.g., gaps can be introduced in the sequence of a first sequence). The nucleotides or amino acids at corresponding positions are then compared, and the percent identity between the two sequences is a function of the number of identical positions shared by the sequences (i.e., % identity=# of identical positions/total # of positions×100). In certain embodiments, the length of a sequence aligned for comparison purposes is at least 30%, preferably at least 40%, more preferably at least 60%, and even more preferably at least 70%, 80% or 90% of the length of the reference sequence. The actual comparison of the two sequences can be accomplished by well-known methods, for example, using a mathematical algorithm. A preferred, non-limiting example of such a mathematical algorithm is described in Karlin et al., Proc. Natl. Acad. Sci. USA, 90:5873-5877 (1993). Such an algorithm is incorporated into the NBLAST and XBLAST programs (version 2.0) as described in Altschul et al., Nucleic Acids Res., 25:389-3402 (1997). When utilizing BLAST and Gapped BLAST programs, the default parameters of the respective programs (e.g., NBLAST) can be used. See http://worldwideweb.ncbi.nlm.nih.gov. In one embodiment, parameters for sequence comparison can be set at score=100, wordlength=12, or can be varied (e.g., W=5 or W=20).

Another preferred non-limiting example of a mathematical algorithm utilized for the comparison of sequences is the algorithm of Myers and Miller, CABIOS (1989). Such an algorithm is incorporated into the ALIGN program (version 2.0) which is part of the CGC sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti (1994) Comput. Appl. Biosci., 10:3-5; and FASTA described in Pearson and Lipman (1988) PNAS, 85:2444-8.

In another embodiment, the percent identity between two amino acid sequences can be accomplished using the GAP program in the CGC software package (available at http://worldwideweb.cgc.com) using either a Blossom 63 matrix or a PAM250 matrix, and a gap weight of 12, 10, 8, 6, or 4 and a length weight of 2, 3, or 4. In yet another embodiment, the percent identity between two nucleic acid sequences can be accomplished using the GAP program in the CGC software package (available at http://worldwideweb.cgc.com), using a gap weight of 50 and a length weight of 3.

The present invention also provides isolated nucleic acid molecules that contain a fragment or portion that hybridizes under highly stringent conditions to a nucleotide sequence comprising a nucleotide sequence selected from SEQ ID NO: 1 and comprising at least one polymorphism as shown in Table 1 and the complement thereof and also provides isolated nucleic acid molecules that contain a fragment or portion that hybridizes under highly stringent conditions to a nucleotide sequence encoding an amino acid sequence selected from SEQ ID NO: 2 or polymorphic variant thereof. The nucleic acid fragments of the invention are at least about 15, preferably at least about 18, 20, 23 or 25 nucleotides, and can be 30, 40, 50, 100, 200 or more nucleotides in length. Longer fragments, for example, 30 or more nucleotides in length, which encode antigenic polypeptides described herein are particularly useful, such as for the generation of antibodies as described below.

Probes and Primers

In a related aspect, the nucleic acid fragments of the invention are used as probes or primers in assays such as those described herein. Hybridization probes of the polymorphic sequences may be used for screening purposes.

“Probes” or “primers” are oligonucleotides that hybridize in a base-specific manner to a complementary strand of nucleic acid molecules. Such probes and primers include polypeptide nucleic acids, as described in Nielsen et al. (1991) Science, 254:1497-1500. As used herein, the term “primer” refers to a single-stranded oligonucleotide which acts as a point of initiation of template-directed DNA synthesis using well-known methods (e.g., PCR, LCR) including, but not limited to those described herein. The appropriate length of the primer depends on the particular use, but typically ranges from about 15 to 30 nucleotides.

Typically, a probe or primer comprises a region of nucleotide sequence that hybridizes to at least about 15, typically about 20-25, and more typically about 40, 50 or 75, consecutive nucleotides of a nucleic acid molecule comprising a contiguous nucleotide sequence having SEQ ID NO: 1 and comprising at least one polymorphism as shown in Table 1 or a sequence encoding an amino acid sequence comprising SEQ ID NO: 2 and comprising at least one polymorphism as shown in Table 1.

Preferably, the hybridizing polynucleotides comprise at least 10, more preferably at least 15 nucleotides in length while a hybridizing polynucleotide of the present invention to be used as a probe preferably comprises at least 20 to 50, at least 100, more preferably at least 200, or most preferably at least 500 nucleotides in length.

In other embodiments, the probe or primer is at least 70% identical to the contiguous nucleotide sequence or to the complement of the contiguous nucleotide sequence, preferably at least 80% identical, more preferably at least 90% identical, even more preferably at least 95% identical, or even capable of selectively hybridizing to the contiguous nucleotide sequence or to the complement of the contiguous nucleotide sequence. Often, the probe or primer further comprises a label, e.g., radioisotope, fluorescent compound, enzyme, or enzyme co-factor.

The polynucleotides of the invention may be useful as probes in Northern or Southern Blot analysis of RNA or DNA preparations, respectively, or can be used as oligonucleotide primers in PCR analysis dependent on their respective size. Also comprised by the invention are hybridizing polynucleotides which are useful for analyzing DNA-Protein interactions via, e.g., electrophoretic mobility shift analysis (EMSA).

The nucleic acid molecules of the invention such as those described herein can be identified and isolated using standard molecular biology techniques and the sequence information provided in SEQ ID NO: 1. For example, nucleic acid molecules can be amplified and isolated by the polymerase chain reaction using synthetic oligonucleotide primers designed based on one or more of the sequences provided in SEQ ID NO: 1 (and optionally comprising at least one polymorphism as shown in Table 1) and/or the complement thereof. See generally PCR Technology: Principles and Applications for DNA Amplification (ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res., 19:4967 (1991); Eckert et al., PCR Methods and Applications, 1:17 (1991); PCR (eds. McPherson et al., IRL Press, Oxford); and U.S. Pat. No. 4,683,202. The nucleic acid molecules can be amplified using cDNA, mRNA or genomic DNA as a template, cloned into an appropriate vector and characterized by DNA sequence analysis.

Other suitable amplification methods include the ligase chain reaction (LCR) (see Wu and Wallace, Genomics, 4:560 (1989), Landegren et al., Science, 241:1077 (1988), transcription amplification (Kwoh et al., Proc. Natl. Acad. Sci. USA, 86:1173 (1989)), and self-sustained sequence replication (Guatelli et al., Proc. Nat. Acad. Sci. USA, 87:1874 (1990)) and nucleic acid based sequence amplification (NASBA), or any other nucleic acid amplification method, followed by the detection of the amplified molecules using techniques well known to those of skill in the art. These detection schemes are especially useful for the detection of nucleic acid molecules if such molecules are present in very low numbers.

The amplified DNA can be radiolabelled and used as a probe for screening a cDNA library derived from human cells, mRNA in zap express, ZIPLOX or other suitable vector. Corresponding clones can be isolated, DNA can obtained following in vivo excision, and the cloned insert can be sequenced in either or both orientations by art recognized methods to identify the correct reading frame encoding a polypeptide of the appropriate molecular weight. For example, the direct analysis of the nucleotide sequence of nucleic acid molecules of the present invention can be accomplished using well-known methods that are commercially available. See, for example, Sambrook et al., Molecular Cloning, A Laboratory Manual (2nd Ed., CSHP, New York 1989); Zyskind et al., Recombinant DNA Laboratory Manual, (Acad. Press, 1988). Using these or similar methods, the polypeptide and the DNA encoding the polypeptide can be isolated, sequenced and further characterized.

Antisense molecules can be useful, for example, in studying variant IRF6 gene regulation. With respect to variant IRF6 gene regulation, such techniques can be used to modulate, for example, the phenotype associated with of Van der Woude syndrome, Poplitieal syndrome, isolated cleft lip and/or palate. Antisense nucleic acid molecules of the invention can be designed using the nucleotide sequences of SEQ ID NO: 1 and comprising at least one polymorphism as shown in Table 1.

In general, the isolated nucleic acid sequences of the invention can be used as molecular weight markers on Southern gels, and as chromosome markers which are labeled to map related gene positions. The nucleic acid sequences can also be used to compare with endogenous DNA sequences in patients to identify genetic disorders (e.g., a predisposition for or susceptibility to an IRF6 dysfunction or dysregulation, e.g., VWS, PPS, CL/P, or any other cleft lip disorder), and as probes, such as to hybridize and discover related DNA sequences or to subtract out known sequences from a sample. The nucleic acid sequences can further be used to derive primers for genetic fingerprinting, to raise anti-polypeptide antibodies using DNA immunization techniques, and as an antigen to raise anti-DNA antibodies or elicit immune responses. Portions or fragments of the nucleotide sequences identified herein (and the corresponding complete gene sequences) can be used in numerous ways as polynucleotide reagents. For example, these sequences can be used to: (i) map their respective genes on a chromosome; and, thus, locate gene regions associated with genetic disease and (ii) identify an individual from a minute biological sample (tissue typing. Additionally, the nucleotide sequences of the invention can be used to identify and express recombinant polypeptides for analysis, characterization or therapeutic use, or as markers for tissues in which the corresponding polypeptide is expressed, either constitutively, during tissue differentiation, or in diseased states. The nucleic acid sequences can additionally be used as reagents in the screening and/or diagnostic assays described herein, and can also be included as components of kits (e.g., reagent kits) for use in the screening and/or diagnostic assays described herein.

Another aspect of the invention pertains to nucleic acid constructs containing a polymorphic nucleic acid molecule comprising the nucleotide as depicted in SEQ ID NO: 1 and comprising at least one polymorphism as shown in Table 1. Yet another aspect of the invention pertains to nucleic acid constructs containing a nucleic acid molecule encoding a polypeptide comprising the amino acid sequence of SEQ ID NO: 2 and at least one polymorphism as shown in Table 1.

It is well known in the art that genes comprise structural elements which encode an amino acid sequence as well as regulatory elements which are involved in the regulation of the expression of said genes. Structural elements are represented by exons which may either encode an amino acid sequence or which may encode for RNA which is not encoding an amino acid sequence but is nevertheless involved in RNA function, e.g., by regulating the stability of the RNA or the nuclear export of the RNA.

Regulatory elements of a gene may comprise promoter elements or enhancer elements both of which could be involved in transcriptional control of gene expression. It is well known in the art that a promoter is to be found upstream of the structural elements of a gene. Regulatory elements such as enhancer elements, however, can be found distributed over the entire locus of a gene. The elements could reside, e.g., in introns, regions of genomic DNA which separate the exons of a gene. Promoter or enhancer elements correspond to polynucleotide fragments which are capable of attracting or binding polypeptides involved in the regulation of the gene comprising said promoter or enhancer elements. For example, polypeptides involved in regulation of the gene comprise the so called transcription factors.

The introns may comprise further regulatory elements which are required for proper gene expression. Introns are usually transcribed together with the exons of a gene resulting in a nascent RNA transcript which contains both, exon and intron sequences. The intron encoded RNA sequences are usually removed by a process known as RNA splicing. However, the process also requires regulatory sequences present on a RNA transcript said regulatory sequences may be encoded by the introns.

In addition, besides their function in transcriptional control and control of proper RNA processing and/or stability, regulatory elements of a gene could be also involved in the control of genetic stability of a gene locus. The elements control, e.g., recombination events or serve to maintain a certain structure of the DNA or the arrangement of DNA in a chromosome.

Therefore, mutations or polymorphisms can occur in exons of a gene which encode an amino acid sequence as discussed supra as well as in regulatory regions which are involved in the above discussed process. The analysis of the nucleotide sequence of a gene locus in its entirety including, e.g., introns is in light of the above desirable. The polymorphisms comprised by the polynucleotides of the present invention can influence the expression level of variant IRF6 protein via mechanisms involving enhanced or reduced transcription of the variant IRF6 gene, stabilization of the gene's RNA transcripts and alteration of the processing of the primary RNA transcripts. Therefore, in a furthermore preferred embodiment of the gene of the invention a nucleotide substitution results in altered expression of the variant gene compared to the corresponding wild type gene.

In another embodiment, the recombinant DNA molecule of the invention can be used for “gene targeting” and/or “gene replacement”, for restoring a mutant gene or for creating a mutant gene via homologous recombination; see for example Mouellic, Proc. Natl. Acad. Sci. USA, 87 (1990), 4712-4716; Joyner, Gene Targeting, A Practical Approach, Oxford University Press. Moreover, certain vectors, expression vectors, are capable of directing the expression of genes to which they are operably linked. In general, expression vectors of utility in recombinant DNA techniques are often in the form of plasmids. However, the invention is intended to include such other forms of expression vectors, such as viral vectors (e.g., replication defective retroviruses, adenoviruses and adeno-associated viruses) that serve equivalent functions.

Yet another aspect of the invention pertains to nucleic acid constructs containing a nucleic acid molecule encoding the amino acid sequence of SEQ ID NO: 2 and comprising at least one polymorphism shown in Table 1 or fragment thereof. The constructs comprise a vector (e.g., an expression vector) into which a sequence of the invention has been inserted in a sense or antisense orientation. Vectors of the invention can be designed for expression of a polypeptide of the invention in a suitable host cell. As used herein, the term “vector” refers to a nucleic acid molecule capable of transporting another nucleic acid to which it has been linked. The vector of the invention may be, for example, a cosmid, a phage, plasmid, viral or retroviral vector. In a preferred embodiment, the vector is a plasmid. Retroviral vectors may be replication competent or replication defective. In the latter case, viral propagation generally will occur only in complementing host cells.

The polynucleotides or genes of the invention may be joined to a vector containing selectable markers for propagation in a host. Generally, a plasmid vector is introduced in a precipate such as a calcium phosphate precipitate, or in a complex with a charged lipid or in carbon-based clusters. Should the vector be a virus, it may be packaged in vitro using an appropriate packing cell line prior to application to host cells.

Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. In a more preferred embodiment of the vector of the invention, the polynucleotide is operatively linked to expression control sequences allowing expression in prokaryotic or eukaryotic cells or isolated fractions thereof.

Expression of the polynucleotides of the invention comprise transcription of the polynucleotide, preferably into a translatable mRNA. Regulatory elements ensuring expression in eukaryotic cells, preferably mammalian cells, are well known to those skilled in the art. They usually comprise regulatory sequences ensuring initiation of transcription and optionally poly-A signals ensuring termination of transcription and stabilization of the transcript. Additional regulatory elements may include transcriptional as well as translational enhancers. The term “regulatory sequence” is intended to include promoters, enhancers and other expression control elements (e.g., polyadenylation signals) involved in gene expression. Such regulatory sequences are described, for example, in Goeddel, Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). Possible regulatory elements permitting expression in prokaryotic host cells comprise, e.g., the lac, trp or tac promoter in E. coli, and examples for regulatory elements permitting expression in eukaryotic host cells are the AOX1 or GAL1 promoter in yeast or the CMV-, SV40-, RSV-promoter (Rous sarcoma virus), CMV-enhancer, SV40-enhancer or a globin intron in mammalian and other animal cells. Beside elements which are responsible for the initiation of transcription such regulatory elements may also comprise transcription termination signals, such as the SV40-poly-A site or the tk-poly-A site, downstream of the polynucleotide. In this context, suitable expression vectors are known in the art such as Okayama-Berg cDNA expression vector pcDV1 (Pharmacia), pCDM8, pRc/CMV, pcDNA1, pcDNA3 (Invitrogen), pSPORT1 (GIBCO BRL), pFastBac (Invitrogen), pYES (Invitrogen). Preferably, said vector is an expression vector and/or a gene transfer or targeting vector. Expression vectors derived from viruses such as retroviruses, vaccinia virus, adeno-associated virus, herpes viruses, or bovine papilloma virus, may be used for delivery of the polynucleotides or vector of the invention into targeted cell population. Methods which are well known to those skilled in the art can be used to construct recombinant viral vectors; see, for example, the techniques described in Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1994).

Alternatively, the polynucleotides and vectors of the invention can be reconstituted into liposomes for delivery to target cells.

Preferred recombinant expression vectors of the invention comprise a nucleic acid molecule of the invention in a form suitable for expression of the nucleic acid molecule in a host cell. This means that the recombinant expression vectors include one or more regulatory sequences, selected on the basis of the host cells to be used for expression, which is operably linked to the nucleic acid sequence to be expressed. Within a recombinant expression vector, “operably or operatively linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell).

It will be appreciated by those skilled in the art that the design of the expression vector can depend on such factors as the choice of the host cell to be transformed and the level of expression of polypeptide desired. The expression vectors of the invention can be introduced into host cells to thereby produce polypeptides, including fusion polypeptides, encoded by nucleic acid molecules as described herein.

Another aspect of the invention relates to a host cell genetically engineered with the polynucleotide of the invention, the gene, or the vector of the invention. The terms “host cell” and “recombinant host cell” are used interchangeably herein. Host cells are useful for producing (i.e., expressing) polypeptides of the invention. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

Vector DNA can be introduced into prokaryotic or eukaryotic cells via conventional transformation or transfection techniques. As used herein, the terms “transformation” and “transfection” are intended to refer to a variety of art-recognized techniques for introducing a foreign nucleic acid molecule (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those that confer resistance to drugs, such as G418, hygromycin and methotrexate. Nucleic acid molecules encoding a selectable marker can be introduced into a host cell on the same vector as the nucleic acid molecule of the invention or can be introduced on a separate vector. Cells stably transfected with the introduced nucleic acid molecule can be identified by drug selection (e.g., cells that have incorporated the selectable marker gene will survive, while the other cells die).

The host cell can be any prokaryotic or eukaryotic cell, such as a bacterial, insect, fungal, plant, animal, mammalian or, preferably, human cell. Preferred fungal cells are, for example, those of the genus Saccharomyces, in particular those of the species S. cerevisiae. The term “prokaryotic” is meant to include all bacteria which can be transformed or transfected with a polynucleotide for the expression of a variant polypeptide of the invention. Prokaryotic hosts may include gram negative as well as gram positive bacteria such as, for example, E. coli, S. typhimurium, Serratia marcescens and Bacillus subtilis. A polynucleotide coding for a mutant form of variant polypeptides of the invention can be used to transform or transfect the host using any of the techniques commonly known to those of ordinary skill in the art.

Methods for preparing fused, operably linked genes and expressing them in bacteria or animal cells are well-known in the art (See e.g., Sambrook, supra). The genetic constructs and methods described therein can be utilized for expression of variant polypeptides of the invention in, e.g., prokaryotic hosts. In general, expression vectors containing promoter sequences which facilitate the efficient transcription of the inserted polynucleotide are used in connection with the host. The expression vector typically contains an origin of replication, a promoter, and a terminator, as well as specific genes which are capable of providing phenotypic selection of the transformed cells.

The transformed prokaryotic hosts can be grown in fermentors and cultured according to techniques known in the art to achieve optimal cell growth. The proteins of the invention can then be isolated from the grown medium, cellular lysats, or cellular membrane fractions. The isolation and purification of the microbially or otherwise expressed polypeptides of the invention may be by any conventional means such as, for example, preparative chromatographic separations and immunological separations such as those involving the use of monoclonal or polyclonal antibodies.

Thus, in a further embodiment the invention relates to a method for producing a molecular variant IRF6 polypeptide or fragment thereof comprising culturing the above described host cell; and recovering said protein or fragment from the culture.

In another embodiment the present invention relates to a method for producing cells capable of expressing a molecular variant IRF6 polypeptide comprising genetically engineering cells with the polymorphic polynucleotides of the invention, the gene of the invention or the vector of the invention.

The cells obtainable by the method of the invention can be used, for example, to test drugs according to the methods described in D. L. Spector, R. D. Goldman, L. A. Leinwand, Cells, a Lab manual, CSH Press 1998. Furthermore, the cells can be used to study known drugs and unknown derivatives thereof for their ability to complement the deficiency caused by mutations in the IRF6 gene. For these embodiments the host cells preferably lack a wild type allele, preferably both alleles of the IRF6 gene and/or have at least one mutated from thereof. Ideally, the gene comprising an allele as comprised by the polynucleotides of the invention could be introduced into the wild type locus by homologous replacement. Alternatively, strong overexpression of a mutated allele over the normal allele and comparison with a recombinant cell line overexpressing the normal allele at a similar level may be used as a screening and analysis system. The cells obtainable by the above-described method may also be used for the screening methods referred to herein.

The invention further provides methods for producing a polypeptide using the host cells of the invention. In one embodiment, the method comprises culturing the host cell of invention (into which a recombinant expression vector encoding a polypeptide of the invention has been introduced) in a suitable medium such that the polypeptide is produced. In another embodiment, the method further comprises isolating the polypeptide from the medium or the host cell.

The host cells of the invention can be used to produce non-human transgenic animals. The animals of the invention provide a useful model for studying IRF6-related disorders. Accordingly, the invention also relates to a method for the production of a transgenic non-human animal. For example, in one embodiment, a polynucleotide or vector of the invention is introduced into a germ cell, an embryonic cell, stem cell, or an egg cell derived therefrom. The non-human animal can be used in accordance with the method of the invention described below and may be a non-transgenic healthy animal, or may have a disease or disorder, preferably a disease caused by at least one mutation in the gene of the invention. Such transgenic animals are well suited for, e.g., pharmacological studies of drugs in connection with variant forms of the above described variant polypeptides since these polypeptides or at least their functional domains are conserved between species in higher eukaryotes, particularly in mammals. Production of transgenic embryos and screening of those can be performed, e.g., as described by A. L. Joyner Ed., Gene Targeting, A Practical Approach (1993), Oxford University Press. The DNA of the embryos can be analyzed using, e.g., Southern blots with an appropriate probe or based on PCR techniques. Methods for generating transgenic animals via embryo manipulation and microinjection, particularly animals such as mice, have become conventional in the art and are described, for example, in U.S. Pat. Nos. 4,736,866 and 4,870,009, U.S. Pat. No. 4,873,191 and in Hogan, Manipulating the Mouse Embryo (Cold Spring Harbor Laboratory Press, Cold Spring Harbor, N.Y., 1986). Methods for constructing homologous recombination vectors and homologous recombinant animals are described further in Bradley (1991) Current Opinion in Bio/Technology, 2:823-829 and in PCT Publication Nos. WO 90/11354, WO 91/01140, WO 92/0968, and WO 93/04169. Clones of the non-human transgenic animals described herein can also be produced according to the methods described in Wilmut et al. (1997) Nature, 385:810-813 and PCT Publication Nos. WO 97/07668 and WO 97/07669.

As used herein, a “transgenic animal” is a non-human animal in which one or more of the cells of the animal includes a transgene. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal in which an endogenous gene has been altered by homologous recombination between the endogenous gene and an exogenous DNA molecule introduced into a cell of the animal, e.g., an embryonic cell of the animal, prior to development of the animal.

A transgenic non-human animal in accordance with the invention may be a transgenic mouse, rat, hamster, dog, monkey, rabbit, pig, frog, nematode such as Caenorhabditis elegans, fruitfly such as Drosophila melanogaster or fish such as torpedo fish or zebrafish comprising a polynucleotide or vector of the invention or obtained by the method described above, preferably wherein said polynucleotide or vector is stably integrated into the genome of the non-human animal, preferably such that the presence of said polynucleotide or vector leads to the expression of the variant polypeptide of the invention. In a preferred embodiment the transgenic non-human animal of the invention is a mouse, a rat or a zebrafish. It may comprise one or several copies of the same or different polynucleotides of the invention. Numerous reports revealed that said animals are particularly well suited as model organisms for the investigation of human maladies. Advantageously, transgenic animals can be easily created using said model organisms, due to the availability of various suitable techniques well known in the art. Accordingly, in this instance, the mammal is preferably a laboratory animal such as a mouse or rat.

The variant polypeptides of this invention have a variety of uses. They can be used to identify proteins which bind or interact with variant IRF6 polypeptides of the invention. They can also be used, for example, to detect the presence of an antibody that binds to these polypeptide(s) or fragment(s) thereof. They may also be used to raise antibodies in a suitable host, which may be, but not limited to, rabbit, mouse, rat, goat, or human, as non-inclusive examples. They are also useful as targets for therapeutic intervention. Variant IRF6 polypeptides of the invention may also be used an agent to screen pharmaceutical candidates (both in vitro and in vivo), for rational (i.e., structure-based) drug design, as well as possible therapeutic uses. Variant IRF6 polypeptides of the invention may also be immobilized on a surface, e.g., a solid support. Such solid supports are useful for screening, e.g. for variant IRF6 polypeptide binding partners.

The invention therefore also relates to isolated IRF6 polypeptides (“variant IRF6 polypeptides”)encoded by variant IRF6 nucleic acid having at least one polymorphism associated with VWS, PPS, CL/P, or any such other cleft-lip disorders and more specifically, having at least one polymorphism as shown in Table 1.

The polypeptides of the invention can be purified to homogeneity. It is understood, however, that preparations in which the polypeptide is not purified to homogeneity are useful. The critical feature is that the preparation allows for the desired function of the polypeptide, even in the presence of considerable amounts of other components. Thus, the invention encompasses various degrees of purity. In one embodiment, the language “substantially free of cellular material” includes preparations of the polypeptide having less than about 30% (by dry weight) other proteins (i.e., contaminating protein), less than about 20% other proteins, less than about 10% other proteins, or less than about 5% other proteins.

For the investigation of the nature of the alterations in the amino acid sequence of the polypeptides of the invention may be used such as RASMOL that are obtainable from the Internet. Furthermore, folding simulations and computer redesign of structural motifs can be performed using other appropriate computer programs (Olszewski, Proteins 25 (1996), 286-299; Hoffman, Comput. Appl. Biosci. 11 (1995), 675-679). Computers can be used for the conformational and energetic analysis of detailed protein models (Monge, J. Mol. Biol. 247 (1995), 995-1012; Renouf, Adv. Exp. Med. Biol. 376 (1995), 37-45). These analyses can be used for the identification of the influence of a particular mutation on binding and/or processing of drugs. Usually, the amino acid substitutions in the amino acid sequence of the protein encoded by the polynucleotide of the invention are due to one or more nucleotide substitutions. In some embodiments, it is preferable that the polymorphism results in an amino acid substitution of Arg at a position corresponding to position 84 of the IRF6 polypeptide (GenBank Accession No. NP_{—}006138.1).

Amino acids that are essential for function can be identified by methods known in the art, such as site-directed mutagenesis or alanine-scanning mutagenesis (Cunningham et al., Science, 244:1081-1085 (1989)). The latter procedure introduces single alanine mutations at every residue in the molecule. The resulting mutant molecules are then tested for biological activity in vitro, or in vitro proliferative activity. Sites that are critical for polypeptide activity can also be determined by structural analysis such as crystallization, nuclear magnetic resonance or photoaffinity labeling (Smith et al., J. Mol. Biol., 224:899-904 (1992); de Vos et al. Science, 255:306-312 (1992)).

The invention also includes polypeptide fragments of the polymorphic or variant polypeptides of the invention. Fragments can be derived from a polypeptide encoded by a nucleic acid molecule comprising the nucleic acid sequence as depicted in SEQ ID NO: 1 and comprising at least one polymorphism as shown in Table 1. As used herein, a fragment comprises at least 6 contiguous amino acids. Useful fragments include those that retain one or more of the biological activities of the polypeptide as well as fragments that can be used as an immunogen to generate polypeptide-specific antibodies. Biologically active fragments include any portion of the full-length polypeptide which confers a biological function on the variant gene product, including ligand binding, and antibody binding. Ligand binding includes binding by nucleic acids, proteins or polypeptides, small biologically active molecules, or large cellular structures. Fragments can be discrete (not fused to other amino acids or polypeptides) or can be within a larger polypeptide. Further, several fragments can be comprised within a single larger polypeptide.

The invention also provides chimeric or fusion polypeptides. These comprise a polypeptide of the invention operatively linked to a heterologous protein or polypeptide having an amino acid sequence not substantially homologous to the polypeptide. “Operatively linked” indicates that the polypeptide and the heterologous protein are fused in-frame. The heterologous protein can be fused to the N-terminus or C-terminus of the polypeptide. In one embodiment the fusion polypeptide does not affect function of the polypeptideper se. For example, the fusion polypeptide can be a GST-fusion polypeptide in which the polypeptide sequences are fused to the C-terminus of the GST sequences. Other types of fusion polypeptides include, but are not limited to, enzymatic fusion polypeptides, for example β (beta)-galactosidase fusions, yeast two-hybrid GAL fusions, poly-His fusions and Ig fusions. Such fusion polypeptides, particularly poly-His fusions, can facilitate the purification of recombinant polypeptide. In certain host cells (e.g., mammalian host cells), expression and/or secretion of a polypeptide can be increased by using a heterologous signal sequence. Therefore, in another embodiment, the fusion polypeptide contains a heterologous signal sequence at its N-tenninus.

EP-A-O 464 533 discloses fusion proteins comprising various portions of immunoglobulin constant regions. The Fc is useful in therapy and diagnosis and thus results, for example, in improved pharmacokinetic properties (EP-A 0232 262). In drug discovery, for example, human proteins have been fused with Fc portions for the purpose of high-throughput screening assays to identify antagonists. Bennett et al., Journal of Molecular Recognition, 8:52-58 (1995) and Johanson et al., The Journal of Biological Chemistry, 270,16:9459-9471 (1995). Thus, this invention also encompasses soluble fusion polypeptides containing a polypeptide of the invention and various portions of the constant regions of heavy or light chains of immunoglobulins of various subclass (IgG, IgM, IgA, IgE).

A chimeric or fusion polypeptide can be produced by standard recombinant DNA techniques. For example, DNA fragments coding for the different polypeptide sequences are ligated together in-frame in accordance with conventional techniques. In another embodiment, the fusion gene can be synthesized by conventional techniques including automated DNA synthesizers. Alternatively, PCR amplification of nucleic acid fragments can be carried out using anchor primers which give rise to complementary overhangs between two consecutive nucleic acid fragments which can subsequently be annealed and re-amplified to generate a chimeric nucleic acid sequence (see Ausubel et al., Current Protocols in Molecular Biology, 1992). Moreover, many expression vectors are commercially available that already encode a fusion moiety (e.g., a GST protein). A nucleic acid molecule encoding a polypeptide of the invention can be cloned into such an expression vector such that the fusion moiety is linked in-frame to the polypeptide.

The isolated variant polypeptide can be purified from cells that naturally express it, purified from cells that have been altered to express it (recombinant), or synthesized using known protein synthesis methods. In one embodiment, the polypeptide is produced by recombinant DNA techniques. For example, a nucleic acid molecule of the invention encoding the polypeptide is cloned into an expression vector, the expression vector introduced into a host cell and the polypeptide expressed in the host cell. The polypeptide can then be isolated from the cells by an appropriate purification scheme using standard protein purification techniques.

In general, polypeptides of the present invention can be used as a molecular weight marker on SDS-PAGE gels or on molecular sieve gel filtration columns using art-recognized methods. The polypeptides of the present invention can be used to raise antibodies or to elicit an immune response. The polypeptides can also be used as a reagent, e.g., a labeled reagent, in assays to quantitatively determine levels of the polypeptide or a molecule to which it binds (e.g., a receptor or a ligand) in biological fluids. The polypeptides can also be used as markers for cells or tissues in which the corresponding polypeptide is preferentially expressed, either constitutively, during tissue differentiation, or in a diseased state. The polypeptides can be used to isolate a corresponding binding partner, e.g., receptor or ligand, such as, for example, in an interaction trap assay, and to screen for peptide or small molecule antagonists or agonists of the binding interaction.

The invention furthermore relates to antibodies that bind specifically to the variant gene products (e.g., protein or polypeptide) of the invention, but not to corresponding reference gene products. The term “antibody” as used herein refers to immunoglobulin molecules and immunologically active portions of immunoglobulin molecules, i.e., molecules that contain an antigen binding site that specifically binds an antigen. Antibodies of the invention (e.g., a monoclonal antibody) can be used to isolate a variant polypeptide of the invention by standard techniques, such as affinity chromatography or immunoprecipitation. A polypeptide-specific antibody can facilitate the purification of natural polypeptide from cells and of recombinantly produced polypeptide expressed in host cells. Moreover, an antibody specific for a variant polypeptide of the invention can be used to detect the polypeptide (e.g., in a cellular lysate, cell supernatant, or tissue sample) in order to evaluate the abundance and pattern of activity or expression of the polypeptide. Antibodies can be used diagnostically to monitor protein levels in tissue as part of a clinical testing procedure, e.g., to, for example, determine the efficacy of a given treatment regimen.

Antibodies against the variant polypeptides of the invention can be prepared by well known methods using a purified protein according to the invention or a (synthetic) fragment derived therefrom as an antigen. In a preferred embodiment of the invention, the antibody is a monoclonal antibody, a polyclonal antibody, a single chain antibody, human or humanized antibody, primatized, chimerized or fragment thereof that specifically binds the variant peptide or polypeptide also including bispecific antibody, synthetic antibody, antibody fragment, such as Fab, Fv or scFv fragments etc., or a chemically modified derivative of any of these.

The term “monoclonal antibody” or “monoclonal antibody composition”, as used herein, refers to a population of antibody molecules that contain only one species of an antigen binding site capable of immunoreacting with a particular epitope of a polypeptide of the invention. A monoclonal antibody composition thus typically displays a single binding affinity for a particular polypeptide of the invention with which it immunoreacts. Monoclonal antibodies can be prepared, for example, by the techniques as originally described in Kohler and Milstein, Nature 256 (1975), 495, and Galfre, Meth. Enzymol. 73 (1981), 3, which comprise the fusion of mouse myeloma cells to spleen cells derived from immunized mammals.

Polyclonal antibodies can be prepared as described above by immunizing a suitable subject with a desired immunogen, e.g., polypeptide of the invention or fragment thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein (1975) Nature, 256:495-497, the human B cell hybridoma technique (Kozbor et al. (1983) Immunol. Today, 4:72), the EBV-hybridoma technique (Cole et al. (1985), Monoclonal Antibodies and Cancer Therapy, Alan R. Liss, Inc., pp. 77-96) or trioma techniques. The technology for producing hybridomas is well known (see generally Current Protocols in Immunology (1994) Coligan et al. (eds.) John Wiley & Sons, Inc., New York, N.Y.). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of the invention.

Any of the many well known protocols used for fusing lymphocytes and immortalized cell lines can be applied for the purpose of generating a monoclonal antibody to a polypeptide of the invention (see, e.g., Current Protocols in Immunology, supra; Galfre et al. (1977) Nature, 266:55052; R. H. Kenneth, in Monoclonal Antibodies: A New Dimension In Biological Analyses, Plenum Publishing Corp., New York, N.Y. (1980); and Lerner (1981) Yale J. Biol. Med., 54:387-402. Moreover, the ordinarily skilled worker will appreciate that there are many variations of such methods that also would be useful.

Alternative to preparing monoclonal antibody-secreting hybridomas, a monoclonal antibody to a variant polypeptide of the invention can be identified and isolated by screening a recombinant combinatorial immunoglobulin library (e.g., an antibody phage display library) with the polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurJZAP.™. Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology, 9:1370-1372; Hay et al. (1992) Hum. Antibod. Hybridomas, 3:81-85; Huse et al. (1989) Science, 246:1275-1281; Griffiths et al. (1993) EMBO J., 12:725-734.

Additionally, recombinant antibodies, such as chimeric and humanized antibodies, which can be made using standard recombinant DNA techniques, are within the scope of the invention. Humanized forms of non-human (e.g., murine) antibodies are chimeric molecules of immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Such chimeric and humanized antibodies can be produced by recombinant DNA techniques known in the art.

Yet another highly efficient means for generating recombinant antibodies is disclosed by Newman, Biotechnology, 10: 1455-1460 (1992). Specifically, this technique results in the generation of primatized antibodies that contain monkey variable domains and human constant sequences. This reference is incorporated by reference in its entirety herein. Moreover, this technique is also described in commonly assigned U.S. Pat. Nos. 5,658,570, 5,693,780 and 5,756,096 each of which is incorporated herein by reference.

Detection of antibodies of the invention can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, .beta.-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include 125I, 131I, 35S or 3H.

Furthermore, antibodies or fragments thereof to the aforementioned polypeptides can be obtained by using methods which are described, e.g., in Harlow and Lane “Antibodies, A Laboratory Manual”, CSH Press, Cold Spring Harbor, 1988. These antibodies can be used, for example, for the immunoprecipitation and immunolocalization of the variant polypeptides of the invention as well as for the monitoring of the presence of said variant polypeptides, for example, in recombinant organisms, and for the identification of compounds interacting with the proteins according to the invention. For example, surface plasmon resonance as employed in the BlAcore system can be used to increase the efficiency of phage antibodies which bind to an epitope of the protein of the invention (Schier, Human Antibodies Hybridomas 7 (1996), 97-105; Malmborg, J. Immunol. Methods 183 (1995), 7-13).

Antibodies which specifically recognize modified amino acids such as phospho Tyrosine residues are well known in the art. Similarly, in accordance with the present invention antibodies which specifically recognize even a single amino acid exchange in an epitope may be generated by the well known methods described supra. In light of the foregoing, in a more preferred embodiment the antibody of the present invention is monoclonal or polyclonal.

Antibodies may be attached, directly or indirectly (e.g., via a linker molecule) to a solid support for use in a diagnostic assay to determine and/or measure the presence of a polymorphic IRF6 polypeptide in a biological sample. Attachment is generally covalent, although it need not be. Solid supports include, but are not limited to, beads (e.g., polystyrene beads, magnetic beads, and the like); plastic surfaces (e.g., polystyrene or polycarbonate multi-well plates typically used in an ELISA or radioimmunoassay (RIA), and the like); sheets, e.g., nylon, nitrocellulose, and the like; and chips, e.g., SiO₂ chips such as those used in microarrays. Accordingly, the invention further provides assay devices comprising antibodies attached to a solid support.

The present invention also pertains to a method of diagnosing or aiding in the diagnosis of VWS, PPS, CL/P, or other such cleft lip disorders associated with the presence of a variant form of the IRF6 gene or gene product in an individual. Diagnostic assays can be designed for assessing IRF6 gene expression, or for assessing activity of IRF6 polypeptides of the invention. In one embodiment, the assays are used in the context of a biological sample (e.g., blood, serum, cells, tissue, synovial fluid) to thereby determine whether an individual is afflicted with VWS, PPS, CL/P, or is at risk for (has a predisposition for or a susceptibility to) developing VWS, PPS, CL/P. The invention also provides for prognostic (or predictive) assays for determining whether an individual is susceptible to developing VWS, PPS, CL/P. For example, alterations in nucleic acids can be assayed in a biological sample. Such assays can be used for prognostic or predictive purpose to thereby prophylactically treat an individual prior to the onset of symptoms associated with VWS, PPS, CL/P. Another aspect of the invention pertains to assays for monitoring the influence of agents (e.g., drugs, compounds or other agents) on the expression or activity of polypeptides of the invention, as well as to assays for identifying agents which bind to IRF6 polypeptides.

In one embodiment of the invention, diagnosis of a susceptibility to VWS, PPS, CL/P, or other such cleft lip disorders is made by detecting a polymorphism in IRF6 as described herein. The polymorphism can be a change, i.e., mutation, in IRF6, such as the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of the gene; duplication of all or a part of the gene; transposition of all or a part of the gene; or rearrangement of all or a part of the gene. More than one such change may be present in a single gene. Such sequence changes cause a difference in the polypeptide encoded by an IRF6 nucleic acid. For example, if the alteration is a frame shift mutation, the frame shift can result in a change in the encoded amino acids, and/or can result in the generation of a premature stop codon, causing generation of a truncated polypeptide. Alternatively, a polymorphism associated with a susceptibility to VWS, PPS, CL/P can be a synonymous alteration in one or more nucleotides (i.e., an alteration that does not result in a change in the polypeptide encoded by an IRF6 nucleic acid). Such a polymorphism may alter splicing sites, affect the stability or transport of mRNA, or otherwise affect the transcription or translation of the gene. An IRF6 nucleic acid that has any of the alterations described above is referred to herein as a “variant nucleic acid.”

Detection of an IRF6 polymorphism by analyzing a polynucleotide sample can be conducted in a number of ways. A test nucleic acid sample can be amplified with primers which amplify a region known to comprise an IRF6 polymorphism(s). Genomic DNA or mRNA can be used directly. Alternatively, the region of interest can be cloned into a suitable vector and grown in sufficient quantity for analysis. The nucleic acid may be amplified by conventional techniques, such as a polymerase chain reaction (PCR), to provide sufficient amounts for analysis. The use of the polymerase chain reaction is described in a variety of publications, including, e.g., “PCR Protocols (Methods in Molecular Biology)” (2000) J. M. S. Bartlett and D. Stirling, eds, Humana Press; and “PCR Applications: Protocols for Functional Genomics” (1999) Innis, Gelfand, and Sninsky, eds., Academic Press. Once the region comprising an IRF6 polymorphism has been amplified, the IRF6 polymorphism can be detected in the PCR product by nucleotide sequencing, by SSCP analysis, or any other method known in the art. In performing SSCP analysis, the PCR product may be digested with a restriction endonuclease that recognizes a sequence within the PCR product generated by using as a template a reference IRF6 sequence, but does not recognize a corresponding PCR product generated by using as a template a variant IRF6 sequence by virtue of the fact that the variant sequence no longer contains a recognition site for the restriction endonuclease. Alternatively, various methods are known in the art that utilize oligonucleotide ligation as a means of detecting polymorphisms. See, e.g., Riley et al. (1990) Nucleic Acids Res. 18:2887-2890; and Delahunty et al. (1996) Am. J. Hum. Genet. 58:1239-1246.

As a non-limiting example, a biological sample from a test subject (a “test sample”) of genomic DNA, RNA, or cDNA, is obtained from an individual suspected of having, being susceptible to or predisposed for, or carrying a defect for, VWS, PPS, CL/P, or other such cleft lip disorders (the “test individual”). The individual can be an adult, child, or fetus. The test sample can be from any source which contains genomic DNA, such as a blood sample, sample of amniotic fluid, sample of cerebrospinal fluid, or tissue sample from skin, muscle, buccal or conjunctival mucosa, placenta, gastrointestinal tract or other organs. A test sample of DNA from fetal cells or tissue can be obtained by appropriate methods, such as by amniocentesis or chorionic villus sampling. The DNA, RNA, or cDNA sample is then examined to determine whether a polymorphism in IRF6 is present. The presence of the polymorphism(s) can be indicated by hybridization of the gene in the genomic DNA, RNA, or cDNA to a nucleic acid probe. A “nucleic acid probe”, as used herein, can be a DNA probe or an RNA probe; the nucleic acid probe contains at least one polymorphism in IRF6. The probe can be any of the nucleic acid molecules described above (e.g., the gene, a fragment, a vector comprising the gene, etc.).

A preferred probe for detecting mRNA or genomic DNA is a labeled nucleic acid probe capable of hybridizing to mRNA or genomic DNA sequences described herein. The nucleic acid probe can be, for example, a full-length nucleic acid molecule, or a portion thereof, such as an oligonucleotide of at least 15, 30, 50, 100, 250 or 500 nucleotides in length and sufficient to specifically hybridize under stringent conditions to appropriate mRNA or genomic DNA. For example, the nucleic acid probe can be all or a portion of SEQ ID NO: 1 which comprises at least one polymorphism shown in Table 1. Other suitable probes for use in the diagnostic assays of the invention are described above (see e.g., probes and primers).

The hybridization sample is maintained under conditions which are sufficient to allow specific hybridization of the nucleic acid probe to variant IRF6. “Specific hybridization”, as used herein, indicates exact hybridization (e.g., with no mismatches). Specific hybridization can be performed under high stringency conditions or moderate stringency conditions, for example, as described above. In a particularly preferred embodiment, the hybridization conditions for specific hybridization are high stringency.

Specific hybridization, if present, is then detected using standard methods. If specific hybridization occurs between the nucleic acid probe and variant IRF6 in the test sample, then IRF6 has the polymorphism that is present in the nucleic acid probe. More than one nucleic acid probe can also be used concurrently in this method. Specific hybridization of any one of the nucleic acid probes is indicative of a polymorphism in IRF6, and is therefore diagnostic for a susceptibility to VWS, PPS, CL/P, or other such cleft lip disorders.

Examples of techniques for detecting differences of at least one nucleotide between two nucleic acids include, but are not limited to, selective oligonucleotide hybridization, selective amplification, or selective primer extension. For example, oligonucleotide probes may be prepared in which the known polymorphic nucleotide is placed centrally (allele-specific probes) and then hybridized to target DNA under conditions which permit hybridization only if a perfect match is found (Saiki et al. (1986) Nature 324:163); Saiki et al (1989) Proc. Natl Acad. Sci USA 86:6230; and Wallace et al. (1979) Nucl. Acids Res. 6:3543). Such allele specific oligonucleotide hybridization techniques may be used for the simultaneous detection of several nucleotide changes in different polymorphic regions of IRF6. For example, oligonucleotides having nucleotide sequences of specific allelic variants are attached to a hybridizing membrane and this membrane is then hybridized with labeled sample nucleic acid. Analysis of the hybridization signal will then reveal the identity of the nucleotides of the sample nucleic acid. Allele specific oligonucleotide is described in more detail below.

Other hybridization methods for diagnosing susceptibility to VWS, PPS, CL/P, or other such cleft lip disorders, hybridization methods include Southern analysis, Northern analysis, or in situ hybridizations, can be used (see Current Protocols in Molecular Biology, Ausubel, F. et al., eds., John Wiley & Sons, including all supplements through 1999). For Northern analysis, a test sample of RNA is obtained from the test individual by appropriate means. Specific hybridization of a nucleic acid probe, as described above, to RNA from the test individual is indicative of a polymorphism in IRF6, and is therefore diagnostic for a susceptibility to VWS, PPS, CL/P, or other such cleft lip disorders. In situ may be performed for use in diagnostic procedures directly upon tissue sections (fixed and/or frozen) of patient tissue obtained from biopsies or resections, such that no nucleic acid purification is necessary. Nucleic acid reagents may be used as probes and/or primers for such in situ procedures (see, for example, Nuovo, G. J., 1992, PCR in situ hybridization: protocols and applications, Raven Press, N.Y.).

Furthermore, the mRNA, cRNA, cDNA or genomic DNA obtained from the subject may be sequenced to identify mutations which may be characteristic fingerprints of mutations in the polynucleotide or the gene of the invention as described below. The present invention further comprises methods wherein such a fingerprint may be generated by RFLPs of DNA or RNA obtained from the subject, optionally the DNA or RNA may be amplified prior to analysis, the methods of which are well known in the art. RNA fingerprints may be performed by, for example, digesting an RNA sample obtained from the subject with a suitable RNA-Enzyme, for example RNase T1, RNase T2 or the like or a ribozyme and, for example, electrophoretically separating and detecting the RNA fragments. Further modifications of the above-mentioned embodiment of the invention can be easily devised by the person skilled in the art, without any undue experimentation from this disclosure. An additional embodiment of the present invention relates to a method wherein said determination is effected by employing an antibody of the invention or fragment thereof. The antibody used in the method of the invention may be labeled with detectable tags such as a histidine flags or a biotin molecule.

Alternatively, a peptide nucleic acid (PNA) probe can be used instead of a nucleic acid probe in the hybridization methods described above. PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, Nielsen, P. E. et al., Bioconjugate Chemistry, 1994, 5, American Chemical Society, p. 1 (1994). The PNA probe can be designed to specifically hybridize to a gene having a polymorphism associated with a susceptibility to VWS, PPS, CL/P, or other such cleft lip disorders. Hybridization of the PNA probe to variant IRF6 is diagnostic for a susceptibility to VWS, PPS, CL/P, or other such cleft lip disorders.

Mutation analysis by restriction digestion can be used to detect a variant IRF6 gene containing a polymorphism(s) as disclosed herein, if the polymorphism in the gene results in the creation or elimination of a restriction site. A test sample containing genomic DNA is obtained from the individual. Polymerase chain reaction (PCR) can be used to amplify the variant IRF6 (and, if necessary, the flanking sequences) in the test sample of genomic DNA from the test individual. RFLP analysis is conducted as described (see, for example, Current Protocols in Molecular Biology, supra). The digestion pattern of the relevant DNA fragment indicates the presence or absence of the mutation or polymorphism in IRF6, and therefore indicates the presence or absence of this susceptibility to VWS, PPS, CL/P, or other such cleft lip disorders.

Sequence analysis can also be used to detect specific polymorphisms in IRF6. A test sample of DNA or RNA is obtained from the test individual. PCR or other appropriate methods can be used to amplify the gene, and/or its flanking sequences, if desired. The sequence of IRF6, or a fragment of the gene, or cDNA, or fragment of the cDNA, or mRNA, or fragment of the mRNA, is determined, using standard methods. The sequence of the gene, gene fragment, cDNA, cDNA fragment, mRNA, or mRNA fragment is compared with the known nucleic acid sequence of the gene, cDNA (e.g., SEQ ID NO: 1 and comprising at least one polymorphism as shown in Table 1) or mRNA, as appropriate. The presence of a polymorphism in IRF6 indicates that the individual has a susceptibility to VWS, PPS, CL/P, or other such cleft lip disorders.

Allele-specific oligonucleotides can also be used to detect the presence of a polymorphism in IRF6, through the use of dot-blot hybridization of amplified oligonucleotides with allele-specific oligonucleotide (ASO) probes (see, for example, Saiki, R. et al., (1986), Nature (London) 324:163-166). An “allele-specific oligonucleotide” (also referred to herein as an “allele-specific oligonucleotide probe”) is an oligonucleotide of approximately 10-50 base pairs, preferably approximately 15-30 base pairs, that specifically hybridizes to IRF6 that contains a polymorphism associated with a susceptibility to VWS, PPS, CL/P, or other such cleft lip disorders. An allele-specific oligonucleotide probe that is specific for particular polymorphisms in IRF6 can be prepared, using standard methods (see Current Protocols in Molecular Biology, supra). To identify polymorphisms in the gene that are associated with a susceptibility to Van der Woude syndrome, Poplitieal pterygium syndrome, isolated cleft lip and/or palate, or other such cleft lip disorders, a test sample of DNA is obtained from the individual. PCR can be used to amplify all or a fragment of IRF6, and its flanking sequences. The DNA containing the amplified IRF6 (or fragment of the gene) is dot-blotted, using standard methods (see Current Protocols in Molecular Biology, supra), and the blot is contacted with the oligonucleotide probe. The presence of specific hybridization of the probe to the amplified IRF6 is then detected. Specific hybridization of an allele-specific oligonucleotide probe to DNA from the individual is indicative of a polymorphism in IRF6, and is therefore indicative of a susceptibility to VWS, PPS, CL/P, or other such cleft lip disorders. An oligonucleotide array can be used. Oligonucleotide arrays typically comprise a plurality of different oligonucleotide probes that are coupled to a surface of a substrate in different known locations. These oligonucleotide arrays can also described as “Genechips™,” have been generally described in the art, for example, U.S. Pat. No. 5,143,854 and PCT patent publication Nos. WO 90/15070 and 92/10092. These arrays can generally be produced using mechanical synthesis methods or light directed synthesis methods which incorporate a combination of photolithographic methods and solid phase oligonucleotide synthesis methods. See Fodor et al., Science, 251:767-777 (1991), Pirrung et al., U.S. Pat. No. 5,143,854 (see also PCT Application No. WO 90/15070) and Fodor et al., PCT Publication No. WO 92/10092 and U.S. Pat. No. 5,424,186, the entire teachings of each of which are incorporated by reference herein. Techniques for the synthesis of these arrays using mechanical synthesis methods are described in, e.g., U.S. Pat. No. 5,384,261, the entire teachings of which are incorporated by reference herein.

Once an oligonucleotide array is prepared, a nucleic acid of interest is hybridized with the array and scanned for polymorphisms. Hybridization and scanning are generally carried out by methods described herein and also in, e.g., Published PCT Application Nos. WO 92/10092 and WO 95/11995, and U.S. Pat. No. 5,424,186, the entire teachings of which are incorporated by reference herein. In brief, a target nucleic acid sequence which includes one or more previously identified polymorphic markers is amplified by well known amplification techniques, e.g., PCR. Typically, this involves the use of primer sequences that are complementary to the two strands of the target sequence both upstream and downstream from the polymorphism. Asymmetric PCR techniques may also be used. Amplified target, generally incorporating a label, is then hybridized with the array under appropriate conditions. Upon completion of hybridization and washing of the array, the array is scanned to determine the position on the array to which the target sequence hybridizes. The hybridization data obtained from the scan is typically in the form of fluorescence intensities as a function of location on the array.

Although primarily described in terms of a single detection block, e.g., for detection of a single polymorphism, arrays can include multiple detection blocks, and thus be capable of analyzing multiple, specific polymorphisms. In alternate arrangements, it will generally be understood that detection blocks may be grouped within a single array or in multiple, separate arrays so that varying, optimal conditions may be used during the hybridization of the target to the array. As a non-limiting example, it may often be desirable to provide for the detection of those polymorphisms that fall within G-C rich stretches of a genomic sequence, separately from those falling in A-T rich segments. This allows for the separate optimization of hybridization conditions for each situation.

Additional description of use of oligonucleotide arrays for detection of polymorphisms can be found, for example, in U.S. Pat. Nos. 5,858,659 and 5,837,832, the entire teachings of which are incorporated by reference herein.

Other methods of nucleic acid analysis can be used to detect polymorphisms in IRF6. Representative methods include direct manual sequencing (Church and Gilbert, (1988), Proc. Natl. Acad. Sci. USA 81:1991-1995; Sanger, F. et al. (1977) Proc. Natl. Acad. Sci. 74:5463-5467; Beavis et al. U.S. Pat. No. 5,288,644); automated fluorescent sequencing; single-stranded conformation polymorphism assays (SSCP) Fischer et al. (1983) Proc Natl Acad Sci USA 80:1579-1583, Orita et al. (1989) Genomics 5:874-879; clamped denaturing gel electrophoresis (CDGE); mobility shift analysis (Orita, M. et al. (1989) Proc. Natl. Acad. Sci. USA 86:2766-2770), restriction enzyme analysis (Flavell et al. (1978) Cell 15:25; Geever, et al. (1981) Proc. Natl. Acad. Sci. USA 78:5081); heteroduplex analysis; chemical mismatch cleavage (CMC) (Cotton et al. (1985) Proc. Natl. Acad. Sci. USA 85:4397-4401); RNase protection assays (Myers, R. M. et al. (1985) Science 230:1242); amplified fragment-length polymorphism (AFLP) Vos et al. (1995) Nucleic Acids Res 23:4407-4414; microsatellite or single-sequence repeat (SSR) Weber J L and May P E (1989) Am J Hum Genet 44:388-396; rapid-amplified polymorphic DNA (RAPD) Williams et al (1990) Nucleic Acids Res 18:6531-6535; sequence tagged site (STS) Olson et al. (1989) Science 245:1434-1435; genetic-bit analysis (GBA) Nikiforov et al (1994) Nucleic Acids Res 22:4167-4175; nick-translation PCR (e.g., TAQMAN™) Lee et al. (1993) Nucleic Acids Res 21:3761-3766; and allele-specific hybridization (ASH) Wallace et al. (1979) Nucleic Acids Res 6:3543-3557, (Sheldon et al. (1993) Clinical Chemistry 39(4):718-719); use of polypeptides which recognize nucleotide mismatches, such as E. coli mutS protein; allele-specific PCR (Gibbs et al. (1989) Nucleic Acids Res 17:2437-2448, Newton et al. (1989) Nucleic Acids Res 17:2503-2516), for example. Each technology has its own particular basis for detecting polymorphisms in DNA sequence.

The following is a general overview of some other techniques which can be used to assay for the polymorphisms of the invention:

PCR Amplification

The most common means for amplification is polymerase chain reaction (PCR), as described in U.S. Pat. Nos. 4,683,195; 4,683,202; and 4,965,188 each of which is hereby incorporated by reference. If PCR is used to amplify the target regions in blood cells, heparinized whole blood should be drawn in a sealed vacuum tube kept separated from other samples and handled with clean gloves. For best results, blood should be processed immediately after collection; if this is impossible, it should be kept in a sealed container at 4° C. until use. Cells in other physiological fluids may also be assayed. When using any of these fluids, the cells in the fluid should be separated from the fluid component by centrifugation.

Tissues should be roughly minced using a sterile, disposable scalpel and a sterile needle (or two scalpels) in a 5 mm Petri dish. Procedures for removing paraffin from tissue sections are described in a variety of specialized handbooks well known to those skilled in the art.

To amplify a target nucleic acid sequence in a sample by PCR, the sequence must be accessible to the components of the amplification system. One method of isolating target DNA is crude extraction which is useful for relatively large samples. Briefly, mononuclear cells from samples of blood, amniocytes from amniotic fluid, cultured chorionic villus cells, or the like are isolated by layering on a sterile Ficoll-Hypaque gradient by standard procedures. Interphase cells are collected and washed three times in sterile phosphate buffered saline before DNA extraction. If testing DNA from peripheral blood lymphocytes, an osmotic shock (treatment of the pellet for 10 sec with distilled water) is suggested, followed by two additional washings if residual red blood cells are visible following the initial washes. This will prevent the inhibitory effect of the heme group carried by hemoglobin on the PCR reaction. If PCR testing is not performed immediately after sample collection, aliquots of 10⁶ cells can be pelleted in sterile Eppendorf tubes and the dry pellet frozen at −20° C. until use.

The cells are resuspended (10⁶ nucleated cells per 100 μl) in a buffer of 50 mM Tris-HCl (pH 8.3), 50 mM KCl 1.5 mM MgCl₂, 0.5% Tween 20, and 0.5% NP40 supplemented with 100 μg/ml of proteinase K. After incubating at 56° C. for 2 hr. the cells are heated to 95° C. for 10 min to inactivate the proteinase K and immediately moved to wet ice (snap-cool). If gross aggregates are present, another cycle of digestion in the same buffer should be undertaken. Ten μl of this extract is used for amplification.

When extracting DNA from tissues, e.g., chorionic villus cells or confluent cultured cells, the amount of the above mentioned buffer with proteinase K may vary according to the size of the tissue sample. The extract is incubated for 4-10 hrs at 50°-60° C. and then at 95° C. for 10 minutes to inactivate the proteinase. During longer incubations, fresh proteinase K should be added after about 4 hr at the original concentration.

When the sample contains a small number of cells, extraction may be accomplished by methods as described in Higuchi, “Simple and Rapid Preparation of Samples for PCR”, in PCR Technology, Ehrlich, H. A. (ed.), Stockton Press, New York, which is incorporated herein by reference. PCR can be employed to amplify target regions in very small numbers of cells (1000-5000) derived from individual colonies from bone marrow and peripheral blood cultures. The cells in the sample are suspended in 20 μl of PCR lysis buffer (10 mM Tris-HCl (pH 8.3), 50 mM KCl, 2.5 mM MgCl₂, 0.1 mg/ml gelatin, 0.45% NP40, 0.45% Tween 20) and frozen until use. When PCR is to be performed, 0.6 μl of proteinase K (2 mg/ml) is added to the cells in the PCR lysis buffer. The sample is then heated to about 60° C. and incubated for 1 hr. Digestion is stopped through inactivation of the proteinase K by heating the samples to 95° C. for 10 min and then cooling on ice.

A relatively easy procedure for extracting DNA for PCR is a salting out procedure adapted from the method described by Miller et al., Nucleic Acids Res. 16:1215 (1988), which is incorporated herein by reference. Mononuclear cells are separated on a Ficoll-Hypaque gradient. The cells are resuspended in 3 ml of lysis buffer (10 mM Tris-HCl, 400 mM NaCl, 2 mM Na₂ EDTA, pH 8.2). Fifty μl of a 20 mg/ml solution of proteinase K and 150 μl of a 20% SDS solution are added to the cells and then incubated at 37° C. overnight. Rocking the tubes during incubation will improve the digestion of the sample. If the proteinase K digestion is incomplete after overnight incubation (fragments are still visible), an additional 50 μl of the 20 mg/ml proteinase K solution is mixed in the solution and incubated for another night at 37° C. on a gently rocking or rotating platform. Following adequate digestion, one ml of a 6M NaCl solution is added to the sample and vigorously mixed. The resulting solution is centrifuged for 15 minutes at 3000 rpm. The pellet contains the precipitated cellular proteins, while the supernatant contains the DNA. The supernatant is removed to a 15 ml tube that contains 4 ml of isopropanol. The contents of the tube are mixed gently until the water and the alcohol phases have mixed and a white DNA precipitate has formed. The DNA precipitate is removed and dipped in a solution of 70% ethanol and gently mixed. The DNA precipitate is removed from the ethanol and air-dried. The precipitate is placed in distilled water and dissolved.

Kits for the extraction of high-molecular weight DNA for PCR include a Genomic Isolation Kit A.S.A.P. (Boehringer Mannheim, Indianapolis, Ind.), Genomic DNA Isolation System (GIBCO BRL, Gaithersburg, Md.), Elu-Quik DNA Purification Kit (Schleicher & Schuell, Keene, N.H.), DNA Extraction Kit (Stratagene, LaJolla, Calif.), TurboGen Isolation Kit (Invitrogen, San Diego, Calif.), and the like. Use of these kits according to the manufacturer's instructions is generally acceptable for purification of DNA prior to practicing the methods of the present invention.

The concentration and purity of the extracted DNA can be determined by spectrophotometric analysis of the absorbance of a diluted aliquot at 260 nm and 280 nm. After extraction of the DNA, PCR amplification may proceed. The first step of each cycle of the PCR involves the separation of the nucleic acid duplex formed by the primer extension. Once the strands are separated, the next step in PCR involves hybridizing the separated strands with primers that flank the target sequence. The primers are then extended to form complementary copies of the target strands. For successful PCR amplification, the primers are designed so that the position at which each primer hybridizes along a duplex sequence is such that an extension product synthesized from one primer, when separated from the template (complement), serves as a template for the extension of the other primer. The cycle of denaturation, hybridization, and extension is repeated as many times as necessary to obtain the desired amount of amplified nucleic acid.

In a particularly useful embodiment of PCR amplification, strand separation is achieved by heating the reaction to a sufficiently high temperature for a sufficient time to cause the denaturation of the duplex but not to cause an irreversible denaturation of the polymerase (see U.S. Pat. No. 4,965,188, incorporated herein by reference). Typical heat denaturation involves temperatures ranging from about 80° C. to 105° C. for times ranging from seconds to minutes. Strand separation, however, can be accomplished by any suitable denaturing method including physical, chemical, or enzymatic means. Strand separation may be induced by a helicase, for example, or an enzyme capable of exhibiting helicase activity. For example, the enzyme RecA has helicase activity in the presence of ATP. The reaction conditions suitable for strand separation by helicases are known in the art (see Kuhn Hoffman-Berling, 1978, CSH-Quantitative Biology, 43:63-67; and Radding, 1982, Ann. Rev. Genetics 16:405-436, each of which is incorporated herein by reference).

Template-dependent extension of primers in PCR is catalyzed by a polymerizing agent in the presence of adequate amounts of four deoxyribonucleotide triphosphates (typically dATP, dGTP, dCTP, and dTTP) in a reaction medium comprised of the appropriate salts, metal cations, and pH buffering systems. Suitable polymerizing agents are enzymes known to catalyze template-dependent DNA synthesis. In some cases, the target regions may encode at least a portion of a protein expressed by the cell. In this instance, mRNA may be used for amplification of the target region. Alternatively, PCR can be used to generate a cDNA library from RNA for further amplification, the initial template for primer extension is RNA. Polymerizing agents suitable for synthesizing a complementary, copy-DNA (cDNA) sequence from the RNA template are reverse transcriptase (RT), such as avian myeloblastosis virus RT, Moloney murine leukemia virus RT, or Thermus thermophilus (Tth) DNA polymerase, a thermostable DNA polymerase with reverse transcriptase activity marketed by Perkin Elmer Cetus, Inc. Typically, the genomic RNA template is heat degraded during the first denaturation step after the initial reverse transcription step leaving only DNA template. Suitable polymerases for use with a DNA template include, for example, E. coli DNA polymerase I or its Klenow fragment, T4 DNA polymerase, Tth polymerase, and Taq polymerase, a heat-stable DNA polymerase isolated from Thermus aquaticus and commercially available from Perkin Elmer Cetus, Inc. The latter enzyme is widely used in the amplification and sequencing of nucleic acids. The reaction conditions for using Taq polymerase are known in the art and are described in Gelfand, 1989, PCR Technology, supra.

Allele Specific PCR

Allele-specific PCR differentiates between target regions differing in the presence of absence of a variation or polymorphism. PCR amplification primers are chosen which bind only to certain alleles of the target sequence. This method is described by Gibbs, Nucleic Acid Res. 17:12427-2448 (1989).

Allele Specific Oligonucleotide Screening Methods

Further diagnostic screening methods employ the allele-specific oligonucleotide (ASO) screening methods, as described by Saiki et al., Nature 324:163-166 (1986). Oligonucleotides with one or more base pair mismatches are generated for any particular allele. ASO screening methods detect mismatches between variant target genomic or PCR amplified DNA and non-mutant oligonucleotides, showing decreased binding of the oligonucleotide relative to a mutant oligonucleotide. Oligonucleotide probes can be designed so that under low stringency, they will bind to both polymorphic forms of the allele, but at high stringency, bind to the allele to which they correspond. Alternatively, stringency conditions can be devised in which an essentially binary response is obtained, i.e., an ASO corresponding to a variant form of the target gene will hybridize to that allele, and not to the wild-type allele.

Ligase Mediated Allele Detection Method

Target regions of a test subject's DNA can be compared with target regions in unaffected and affected family members by ligase-mediated allele detection. See Landegren et al., Science 241:107-1080 (1988). Ligase may also be used to detect point mutations in the ligation amplification reaction described in Wu et al., Genomics 4:560-569 (1989). The ligation amplification reaction (LAR) utilizes amplification of specific DNA sequence using sequential rounds of template dependent ligation as described in Wu, supra, and Barany, Proc. Nat. Acad. Sci. 88:189-193 (1990).

Denaturing Gradient Gel Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution. DNA molecules melt in segments, termed melting domains, under conditions of increased temperature or denaturation. Each melting domain melts cooperatively at a distinct, base-specific melting temperature (Tₘ). Melting domains are at least 20 base pairs in length, and may be up to several hundred base pairs in length.

Differentiation between alleles based on sequence specific melting domain differences can be assessed using polyacrylamide gel electrophoresis, as described in Chapter 7 of Erlich, ed., PCR Technology, “Principles and Applications for DNA Amplification”, W.H. Freeman and Co., New York (1992), the contents of which are hereby incorporated by reference.

Generally, a target region to be analyzed by denaturing gradient gel electrophoresis is amplified using PCR primers flanking the target region. The amplified PCR product is applied to a polyacrylamide gel with a linear denaturing gradient as described in Myers et al., Meth. Enzymol. 155:501-527 (1986), and Myers et al., in Genomic Analysis, A Practical Approach, K. Davies Ed. IRL Press Limited, Oxford, pp. 95-139 (1988), the contents of which are hereby incorporated by reference. The electrophoresis system is maintained at a temperature slightly below the Tm of the melting domains of the target sequences.

In an alternative method of denaturing gradient gel electrophoresis, the target sequences may be initially attached to a stretch of GC nucleotides, termed a GC clamp, as described in Chapter 7 of Erlich, supra. Preferably, at least 80% of the nucleotides in the GC clamp are either guanine or cytosine. Preferably, the GC clamp is at least 30 bases long. This method is particularly suited to target sequences with high Tₘ's.

Generally, the target region is amplified by the polymerase chain reaction as described above. One of the oligonucleotide PCR primers carries at its 5′ end, the GC clamp region, at least 30 bases of the GC rich sequence, which is incorporated into the 5′ end of the target region during amplification. The resulting amplified target region is run on an electrophoresis gel under denaturing gradient conditions as described above. DNA fragments differing by a single base change will migrate through the gel to different positions, which may be visualized by ethidium bromide staining.

Temperature Gradient Gel Electrophoresis

Temperature gradient gel electrophoresis (TGGE) is based on the same underlying principles as denaturing gradient gel electrophoresis, except the denaturing gradient is produced by differences in temperature instead of differences in the concentration of a chemical denaturant. Standard TGGE utilizes an electrophoresis apparatus with a temperature gradient running along the electrophoresis path. As samples migrate through a gel with a uniform concentration of a chemical denaturant, they encounter increasing temperatures. An alternative method of TGGE, temporal temperature gradient gel electrophoresis (TTGE or tTGGE) uses a steadily increasing temperature of the entire electrophoresis gel to achieve the same result. As the samples migrate through the gel the temperature of the entire gel increases, leading the samples to encounter increasing temperature as they migrate through the gel. Preparation of samples, including PCR amplification with incorporation of a GC clamp, and visualization of products are the same as for denaturing gradient gel electrophoresis.

Single-Strand Conformation Polymorphism Analysis

Target sequences or alleles at the variant IRF6 loci can be differentiated using single-strand conformation polymorphism analysis, which identifies base differences by alteration in electrophoretic migration of single-stranded PCR products, as described in Orita et al., Proc. Nat. Acad. Sci. 85:2766-2770 (1989). Amplified PCR products can be generated as described above, and heated or otherwise denatured, to form single-stranded amplification products. Single-stranded nucleic acids may refold or form secondary structures which are partially dependent on the base sequence. Thus, electrophoretic mobility of single-stranded amplification products can detect base-sequence difference between alleles or target sequences.

Chemical or Enzymatic Cleavage of Mismatches

Differences between target sequences can also be detected by differential chemical cleavage of mismatched base pairs, as described in Grompe et al., Am. J. Hum. Genet. 48:212-222 (1991). In another method, differences between target sequences can be detected by enzymatic cleavage of mismatched base pairs, as described in Nelson et al., Nature Genetics 4:11-18 (1993). Briefly, genetic material from an individual and an affected family member may be used to generate mismatch free heterohybrid DNA duplexes. As used herein, “heterohybrid” means a DNA duplex strand comprising one strand of DNA from one individual, and a second DNA strand from another individual, usually an individual differing in the phenotype for the trait of interest. Positive selection for heterohybrids free of mismatches allows determination of small insertions, deletions or other polymorphisms that may be associated with IRF6 polymorphisms.

Non-Gel Systems

Other possible techniques include non-gel systems such as TAQMAN™ (Perkin Elmer). In this system, oligonucleotide PCR primers are designed that flank the mutation in question and allow PCR amplification of the region. A third oligonucleotide probe is then designed to hybridize to the region containing the base subject to change between different alleles of the gene. This probe is labeled with fluorescent dyes at both the 5′ and 3′ ends. These dyes are chosen such that while in this proximity to each other the fluorescence of one of them is quenched by the other and cannot be detected. Extension by Taq DNA polymerase from the PCR primer positioned 5′ on the template relative to the probe leads to the cleavage of the dye attached to the 5′ end of the annealed probe through the 5′ nuclease activity of the Taq DNA polymerase. This removes the quenching effect allowing detection of the fluorescence from the dye at the 3′ end of the probe. The discrimination between different DNA sequences arises through the fact that if the hybridization of the probe to the template molecule is not complete, i.e., there is a mismatch of some form, the cleavage of the dye does not take place. Thus, only if the nucleotide sequence of the oligonucleotide probe is completely complimentary to the template molecule to which it is bound will quenching be removed. A reaction mix can contain two different probe sequences each designed against different alleles that might be present thus allowing the detection of both alleles in one reaction.

Yet another technique includes an Invader Assay, which includes isothermic amplification that relies on a catalytic release of fluorescence. See Third Wave Technology at worldwideweb.twt.com.

Non-PCR Based DNA Diagnostics

The identification of a DNA sequence linked to variant IRF6 can be made without an amplification step, based on polymorphisms including restriction fragment length polymorphisms in an individual and a family member. Hybridization probes are generally oligonucleotides which bind through complementary base pairing to all or part of a target nucleic acid. Probes typically bind target sequences lacking complete complementarity with the probe sequence depending on the stringency of the hybridization conditions. The probes are preferably labeled directly or indirectly, such that by assaying for the presence or absence of the probe, one can detect the presence or absence of the target sequence. Direct labeling methods include radioisotope labeling, such as with P³² or S³⁵. Indirect labeling methods include fluorescent tags, biotin complexes which may be bound to avidin or streptavidin, or peptide or protein tags. Visual detection methods include photoluminescents, Texas red, rhodamine and its derivatives, red leuco dye and 3,3′,5,5′-tetramethylbenzidine (TMB), fluorescein, and its derivatives, dansyl, umbelliferone and the like or with horse radish peroxidase, alkaline phosphatase and the like.

Hybridization probes include any nucleotide sequence capable of hybridizing to the subject's chromosome where IRF6 resides, and thus defining a genetic marker linked to IRF6 including a restriction fragment length polymorphism, a hypervariable region, repetitive element, or a variable number tandem repeat. Hybridization probes can be any gene or a suitable analog. Further suitable hybridization probes include exon fragments or portions of cDNAs or genes known to map to the relevant region of the chromosome.

Preferred tandem repeat hybridization probes for use according to the present invention are those that recognize a small number of fragments at a specific locus at high stringency hybridization conditions, or that recognize a larger number of fragments at that locus when the stringency conditions are lowered.

One or more additional restriction enzymes and/or probes and/or primers can be used. Additional enzymes, constructed probes, and primers can be determined by routine experimentation by those of ordinary skill in the art and are intended to be within the scope of the invention.

In one embodiment of the invention, diagnosis of a susceptibility to VWS, PPS, CL/P, or other such cleft lip disorders can also be made by examining activity a variant IRF6 polypeptide, by a variety of methods, including enzyme linked immunosorbent assays (ELISAs), Western blots, immunoprecipitations and immunofluorescence all of which are known in the art. Other various means of examining the activity a polypeptide, described herein, encoded by variant IRF6 can be used, including spectroscopy, colorimetry, electrophoresis, isoelectric focusing, and immunoassays (e.g., David et al., U.S. Pat. No. 4,376,110) such as immunoblotting (see also Current Protocols in Molecular Biology, particularly chapter 10). For example, an antibody capable of binding to a variant polypeptide (e.g., as described above), preferably an antibody with a detectable label, can be used. Antibodies can be polyclonal or monoclonal. An intact antibody, or a fragment thereof (e.g., Fab or Fv or scFv) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

Western blotting analysis, using an antibody as described above that specifically binds to a polypeptide encoded by a mutant IRF6 gene, or an antibody that specifically binds to a polypeptide encoded by a non-mutant gene, can be used to identify the presence in a test sample of a polypeptide encoded by a polymorphic or mutant IRF6, or the absence in a test sample of a polypeptide encoded by a non-polymorphic or non-mutant gene. The presence of a polypeptide encoded by a polymorphic or mutant gene, or the absence of a polypeptide encoded by a non-polymorphic or non-mutant gene, is diagnostic for a susceptibility to VWS, PPS, CL/P, or other such cleft lip disorders.

In another embodiment, the level or amount of polypeptide encoded by variant IRF6 in a test sample is compared with the level or amount of the polypeptide encoded by IRF6 in a control sample. A level or amount of the polypeptide in the test sample that is higher or lower than the level or amount of the polypeptide in the control sample, such that the difference is statistically significant, is indicative of an alteration in the expression of the polypeptide encoded by IRF6, and is diagnostic for a susceptibility to VWS, PPS, CL/P, or other such cleft lip disorders.

In another embodiment, a method of diagnosing an IRF6 dysfunction or dysregulation in a subject, said method comprises obtaining a biological sample from said subject; analyzing the IRF6 nucleic acid in said sample obtained from said subject; and determining the presence of at least one mutation as set forth in Table 1 of IRF6 in said subject, wherein the presence of said mutation is indicative of said subject having an IRF6-dysfunction or dysregulation.

Yet in another embodiment, a method of diagnosing a susceptibility or propensity to Van der Woude syndrome, Poplitieal pterygium syndrome, or isolated cleft lip and/or palate in a subject, comprising obtaining a biological sample from said subject, wherein said sample comprises the IRF6 nucleic acid; and detecting a polymorphism in said IRF6 nucleic acid, wherein the presence of a polymorphism in is indicative of said subject being susceptible to or having a propensity for Van der Woude syndrome, Poplitieal pterygium syndrome, or isolated cleft lip and/or palate.

The invention also contemplates methods of diagnosing a susceptibility to Van der Woude syndrome, Poplitieal pterygium syndrome, or isolated cleft lip and/or palate, or other such cleft lip disorders in an individual, comprising screening for an at-risk haplotype in the IRF6 gene that is more frequently present in an individual susceptible to VWS, PPS, CL/P, or other such cleft lip disorders (affected), compared to the frequency of its presence in a healthy individual (control), wherein the presence of a haplotype is indicative of susceptibility to Van der Woude syndrome, Poplitieal pterygium syndrome, or isolated cleft lip and/or palate. Standard techniques for genotyping for the presence of SNPs and/or microsatellite markers that are associated with VWS, PPS, CL/P, or other such cleft lip disorders can be used, such as fluorescent based techniques (Chen et al. (1999) Genome Res. 9:492), PCR, LCR, Nested PCR, kinetic thermal cycling to determine whether the patient is heterozygous or homozygous for a particular haplotype and other techniques for nucleic acid amplification.

The invention also contemplates kits useful in the methods of diagnosis comprise components useful in any of the methods described herein, including for example, hybridization probes, restriction enzymes (e.g., for RFLP analysis), allele-specific oligonucleotides, antibodies which bind to mutant or to non-mutant (native) IRF6 polypeptide (e.g., to SEQ ID NO:2 and comprising at least one polymorphism as shown in Table 1), means for amplification of nucleic acids comprising variant IRF6, or means for analyzing the nucleic acid sequence of IRF6 or for analyzing the amino acid sequence of an IRF6 polypeptide, etc.

The invention provides methods (also referred to herein as “screening assays”) for identifying the presence of a nucleotide that hybridizes to a nucleic acid of the invention, as well as for identifying the presence of a polypeptide encoded by a nucleic acid of the invention. These methods may be practiced in a variety of embodiments. Screening assays are useful at identifying agents that may affect the expression of variant polynucleotides and disclosed herein or the activity of the variant polypeptides disclosed herein, based on among other factors, the agents ability to modulate the variant nucleic acids activity and or expression.

In one embodiment, the presence (or absence) of a nucleic acid molecule of interest (e.g., a nucleic acid that has significant homology with a nucleic acid of the invention) in a sample can be assessed by contacting the sample with a nucleic acid comprising a nucleic acid of the invention (e.g., a nucleic acid having the sequence of SEQ ID NO: 1 and comprising at least one polymorphism as shown in Table 1, or a nucleic acid encoding an amino acid having the sequence of SEQ ID NO: 2 or a fragment, under stringent conditions as described above, and then assessing the sample for the presence (or absence) of hybridization. In a preferred embodiment, high stringency conditions are conditions appropriate for selective hybridization. In another embodiment, a sample containing the nucleic acid molecule of interest is contacted with a nucleic acid containing a contiguous nucleotide sequence (e.g., a primer or a probe as described above) that is at least partially complementary to a part of the nucleic acid molecule of interest (e.g., a variant IRF6 nucleic acid), and the contacted sample is assessed for the presence or absence of hybridization. In a preferred embodiment, the nucleic acid containing a contiguous nucleotide sequence is completely complementary to a part of the nucleic acid molecule of interest. In any of these embodiment, all or a portion of the nucleic acid of interest can be subjected to amplification prior to performing the hybridization.

In another embodiment, the presence (or absence) of a polypeptide of interest, such as a polypeptide of the invention or a fragment or variant thereof, in a sample can be assessed by contacting the sample with an antibody that specifically hybridizes to the polypeptide of interest (e.g., an antibody such as those described above), and then assessing the sample for the presence (or absence) of binding of the antibody to the polypeptide of interest.

In another embodiment, the invention provides methods for identifying agents/compounds (e.g., fusion proteins, polypeptides, peptidomimetics, prodrugs, receptors, binding agents, antibodies, small molecules or other drugs, or ribozymes) which alter (e.g., increase or decrease) the activity of the variant polypeptides of the invention, or which otherwise interact with the polypeptides of the invention. For example, such agents can be agents which bind to polypeptides described herein (e.g., IFR6 binding agents); which have a stimulatory or inhibitory effect on, for example, activity of polypeptides of the invention; or which change (e.g., enhance or inhibit) the ability of the polypeptides of the invention to interact with IFR6 binding agents (e.g., receptors or other binding agents); or which alter posttranslational processing of the IFR6 variant polypeptide (e.g., agents that alter proteolytic processing to direct the polypeptide from where it is normally synthesized to another location in the cell, such as the cell surface; agents that alter proteolytic processing such that more polypeptide is released from the cell, etc.).

The invention provides assays for screening candidate or test agents that bind to or modulate the activity of polypeptides of the invention, as well as agents identifiable by the assays. Test agents/compounds can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to polypeptide libraries, while the other four approaches are applicable to polypeptide, non-peptide oligomer or small molecule libraries of compounds (Lam, K. S. (1997) Anticancer Drug Des., 12:145.

In one embodiment, to identify agents which alter the activity of an IRF6 variant polypeptide, a cell, cell lysate, or solution containing or expressing an IRF6 variant polypeptide can be contacted with an agent to be tested; alternatively, the polypeptide can be contacted directly with the agent to be tested. The level (amount) of variant polypeptide activity is assessed (e.g., the level (amount) of IRF6 variant polypeptide activity is measured, either directly or indirectly), and is compared with the level of activity in a control (i.e., the level of activity of a wild-type IRF6 polypeptide in the absence of the agent to be tested). If the level of the activity in the presence of the agent differs, by an amount that is statistically significant, from the level of the activity in the absence of the agent, then the agent is an agent that alters the activity of IRF6 polypeptide. An increase in the level of variant IRF6 polypeptide activity relative to a control, indicates that the agent is an agent that enhances (is an agonist of) variant IRF6 polypeptide activity. Similarly, a decrease in the level of variant IRF6 polypeptide activity relative to a control, indicates that the agent is an agent that inhibits (is an antagonist of) variant IRF6 polypeptide activity. In another embodiment, the level of activity of a variant IRF6 polypeptide activity in the presence of the agent to be tested is compared with a control level that has previously been established. A level of the activity in the presence of the agent that differs from the control level by an amount that is statistically significant indicates that the agent alters variant IRF6 polypeptide activity.

The present invention also relates to an assay for identifying agents which alter the expression of the variant IRF6 gene (e.g., antisense nucleic acids, fusion proteins, polypeptides, peptidomimetics, prodrugs, receptors, binding agents, antibodies, small molecules or other drugs, or ribozymes) which alter (e.g., increase or decrease) expression (e.g., transcription or translation) of the gene or which otherwise interact with the nucleic acids described herein, as well as agents identifiable by the assays. For example, a solution containing a nucleic acid encoding IRF6 polypeptide (e.g., variant IRF6 gene) can be contacted with an agent to be tested. The solution can comprise, for example, cells containing the nucleic acid or cell lysate containing the nucleic acid; alternatively, the solution can be another solution which comprises elements necessary for transcription/translation of the nucleic acid. Cells not suspended in solution can also be employed, if desired. The level and/or pattern of variant IRF6 expression (e.g., the level and/or pattern of mRNA or of protein expressed) is assessed, and is compared with the level and/or pattern of expression in a control (i.e., the level and/or pattern of the IRF6 expression in the absence of the agent to be tested). If the level and/or pattern in the presence of the agent differ, by an amount or in a manner that is statistically significant, from the level and/or pattern in the absence of the agent, then the agent is an agent that alters the expression of variant IRF6. Enhancement of variant IRF6 expression indicates that the agent is an agonist of variant IRF6 activity. Similarly, inhibition of variant IRF6 expression indicates that the agent is an antagonist of IRF6 activity.

In another embodiment, the level and/or pattern of variant IRF6 polypeptide in the presence of the agent to be tested, is compared with a control level and/or pattern that has previously been established. A level and/or pattern in the presence of the agent that differs from the control level and/or pattern by an amount or in a manner that is statistically significant indicates that the agent alters variant IRF6 levels and/or pattern.

In another embodiment of the invention, agents which alter the expression of the variant IRF6 gene or which otherwise interact with the nucleic acids described herein, can be identified using a cell, cell lysate, or solution containing a nucleic acid encoding the promoter region of the variant IRF6 gene operably linked to a reporter gene. After contact with an agent to be tested, the level of expression of the reporter gene (e.g., the level of mRNA or of protein expressed) is assessed, and is compared with the level of expression in a control (i.e., the level of the expression of the reporter gene in the absence of the agent to be tested). If the level in the presence of the agent differs, by an amount or in a manner that is statistically significant, from the level in the absence of the agent, then the agent is an agent that alters the expression of variant IRF6 gene, as indicated by its ability to alter expression of a gene that is operably linked to the variant IRF6 gene promoter. Enhancement of the expression of the reporter indicates that the agent is an agonist of variant IRF6 gene activity. Similarly, inhibition of the expression of the reporter indicates that the agent is an antagonist of variant IRF6 gene activity. In another embodiment, the level of expression of the reporter in the presence of the agent to be tested, is compared with a control level that has previously been established. A level in the presence of the agent that differs from the control level by an amount or in a manner that is statistically significant indicates that the agent alters IRF6 expression.

In other embodiments of the invention, assays can be used to assess the impact of a test agent on the activity of a variant polypeptide in relation to an IRF6 binding agent. For example, a cell that expresses a compound that interacts with IRF6 (herein referred to as a “IRF6 binding agent”, which can be a polypeptide or other molecule that interacts with a variant IRF6 polypeptide, such as a receptor) is contacted with variant IRF6 polypeptide in the presence of a test agent, and the ability of the test agent to alter the interaction between the variant polypeptide and the IRF6 binding agent is determined. Alternatively, a cell lysate or a solution containing the IRF6 binding agent can be used. An agent which binds to a variant IRF6 polypeptide of the invention or the IRF6 binding agent can alter the interaction by interfering with, or enhancing the ability of variant IRF6 polypeptide to bind to, associate with, or otherwise interact with the IRF6 binding agent. Determining the ability of the test agent to bind to the variant IRF6 polypeptide or an IRF6 binding agent can be accomplished, for example, by coupling the test agent with a radioisotope or enzymatic label such that binding of the test agent to the polypeptide can be determined by detecting the labeled with ¹²⁵I, ³⁵S, ¹⁴C, or ³H, either directly or indirectly, and the radioisotope detected by direct counting of radioemrnmission or by scintillation counting. Alternatively, test agents can be enzymatically labeled with, for example, horseradish peroxidase, alkaline phosphatase, or luciferase, and the enzymatic label detected by determination of conversion of an appropriate substrate to product. It is also within the scope of this invention to determine the ability of a test agent to interact with the polypeptide without the labeling of any of the interactants.

In more than one embodiment of the methods disclosed herein, it may be desirable to immobilize the molecular species of interest, e.g., either variant IRF6 gene, an IRF6 binding agent, a vector comprising a polymorphic IRF6 polynucleotide of the invention, an antibody as described herein, or a host cell as described herein, or other components of the assay on a solid support. A solid support is may be used as a support capable of binding, for example, an antigen or antibody. A solid support can also be used to facilitate separation of complexed from uncomplexed forms of one or both of the molecular species of interest, as well as to accommodate automation of the assay.

The term “solid support” as used herein refers to a flexible or non-flexible support that is suitable for carrying said immobilized targets. The solid support may be homogenous or inhomogeneous. For example, the solid support may consist of different materials having the same or different properties with respect to flexibility and immobilization, for instance, or said solid support may consist of one material exhibiting a plurality of properties also comprising flexibility and immobilization properties. The solid support may comprise glass-, polypropylene-or silicon-chips, membranes oligonucleotide-conjugated beads or bead arrays.

The term “immobilized” as used herein means that the molecular species of interest is fixed to a solid support, preferably covalently linked thereto. This covalent linkage can be achieved by different means depending on the molecular nature of the molecular species. Moreover, the molecular species may be also fixed on the solid support by electrostatic forces, hydrophobic or hydrophilic interactions or Van-der-Waals forces.

The above described physio-chemical interactions typically occur in interactions between molecules. For example, biotinylated polypeptides may be fixed on an avidincoated solid support due to interactions of the above described types. Further, polypeptides such as antibodies may be fixed on an antibody coated solid support. Moreover, the immobilization is dependent on the chemical properties of the solid support. For example, the nucleic acid molecules can be immobilized on a membrane by standard techniques such as UV-crosslinking or heat.

In a preferred embodiment of the invention said solid support is a membrane, a glass-or poylpropylene-or silicon-chip, are membranes oligonucleotide-conjugated beads or a bead array, which is assembled on an optical filter substrate.

This invention further pertains to novel agents identified by the above-described screening assays. Accordingly, it is within the scope of this invention to further use an agent identified as described herein in an appropriate animal model. For example, an agent identified as described herein (e.g., a test agent that is a modulating agent, an antisense nucleic acid molecule, a specific antibody, or a polypeptide-binding agent) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein. In addition, an agent identified as described herein can be used to alter activity of a variant polypeptide encoded by IRF6, or to alter expression of variant IRF6 gene, by contacting the polypeptide or the gene (or contacting a cell comprising the polypeptide or the gene) with the agent identified as described herein.

In a further embodiment, the invention relates to a method of identifying and obtaining an inhibitor of the activity of a polypeptide, or a derivative, or fragment thereof comprising the amino acid sequence of SEQ ID NO:2 which comprises at least one polymorphism as shown in Table 1, comprising contacting a polypeptide, or a derivative, or fragment thereof comprising the amino acid sequence of SEQ ID NO:2 which comprises at least one polymorphism as shown in Table 1 with a test agent for inhibiting activity in the presence of compounds that provide a detectable symbol in response to test agent activity; and detecting the presence or absence of a signal or increase or decrease of a signal generated from inhibiting activity, wherein the absence or decrease of the signal is indicative inhibiting activity of said polypeptide.

In a further embodiment, the invention relates to a method of identifying and obtaining an inhibitor or activator to a variant IRF6 polypeptide of claim 4 (e.g., a polypeptide having amino acid sequence as depicted in SEQ ID NO:2 and comprising at least one polymorphism as shown in Table 1), wherein said inhibitor or activator modulates the activity of said polypeptide comprising: contacting polypeptide having amino acid sequence as depicted in SEQ ID NO:2 and comprising at least one polymorphism as shown in Table 1 with a first molecule known to be bound by the protein to form a first complex of said protein and said first molecule; contacting said first complex with a candidate compound to be screened; and measuring whether said compound displaces said first molecule from said first complex.

Advantageously, in said method said measuring step comprises measuring the formation of a second complex of said protein and said compound. Preferably, said measuring step comprises measuring the amount of said first molecule that is not bound to the protein. In a particularly preferred embodiment of the above-described method said first molecule is an agonist or antagonist or a substrate and or an inhibitor and/or a modulator of the polypeptide of the invention. Furthermore, it is preferred that in the method of the invention said first molecule is labeled, e.g., with a radioactive or fluorescent label.

In this instance the term “compound” in a method of the invention includes a single substance or a plurality of substances which may or may not be identical. The compound(s) may be chemically synthesized or produced via microbial fermentation but can also be comprised in, for example, samples, e.g., cell extracts from, e.g., plants, animals or microorganisms. Furthermore, the compounds may be known in the art but hitherto not known to be useful as an inhibitor, respectively. The plurality of compounds may be, e.g., added to the culture medium or injected into a cell or nonhuman animal of the invention. If a sample containing (a) compound(s) is identified in the method of the invention, then it is either possible to isolate the compound from the original sample identified as containing the compound, in question or further subdivide the original sample, for example, if it consists of a plurality of different compounds, so as to reduce the number of different substances per sample and repeat the method with the subdivisions of the original sample. It can then be determined whether said sample or compound displays the desired properties, for example, by the methods described herein or in the literature (Spector et al., Cells manual; see supra).

Depending on the complexity of the samples, the steps described above can be performed several times, preferably until the sample identified according to the method of the invention only comprises a limited number of or only one substance(s). Preferably said sample comprises substances of similar chemical and/or physical properties, and most preferably said substances are identical. The methods of the present invention can be easily performed and designed by the person skilled in the art, for example in accordance with other cell based assays described in the prior art or by using and modifying the methods as described herein. Furthermore, the person skilled in the art will readily recognize which further compounds may be used in order to perform the methods of the invention, for example, enzymes, if necessary, that convert a certain compound into a precursor. Such adaptation of the method of the invention is well within the skill of the person skilled in the art and can be performed without undue experimentation.

In particular, such tests are useful to add in predicting whether a given drug will interact in an individual carrying a variant IRF6 gene as described herein. In addition heterologous expression systems such as yeast can be used in order to study the stability, binding properties and catalytic activities of the gene products of the variant IRF6 gene compared to the corresponding wild type IRF6 gene product. As mentioned before, the IRF6 and the molecular variant IRF6 gene and their gene products, particularly when employed in the above described methods, can be used for pharmacological and toxicological studies of the metabolism of drugs.

Agents/compounds which can be used in accordance with the invention include peptides, proteins, nucleic acids, antibodies, small organic compounds, ligands, peptidomimetics, PNAs and the like. Said compounds may act as agonists or antagonists of the invention. The compounds can also be functional derivatives or analogues of known drugs. Methods for the preparation of chemical derivatives and analogues are well known to those skilled in the art and are described in, for example, Beilstein, Handbook of Organic Chemistry, Springer edition New York Inc., 175 Fifth Avenue, New York, N.Y. 10010 U. S. A. and Organic Synthesis, Wiley, N.Y., USA. Furthermore, the derivatives and analogues can be tested for their effects according to methods known in the art or as described. Furthermore, peptide mimetics and/or computer aided design of appropriate drug derivatives and analogues can be used, for example, according to the methods described below. Such analogs comprise molecules may have as the basis structure of known IRF6 substrates and/or inhibitors and/or modulators.

Appropriate computer programs can be used for the identification of interactive sites of a putative inhibitor and the IRF6 protein of the invention by computer assistant searches for complementary structural motifs (Fassina, Immunomethods 5 (1994), 114-120). Further appropriate computer systems for the computer aided design of protein and peptides are described in the prior art, for example, in Berry, Biochem. Soc. Trans. 22 (1994), 1033-1036; Wodak, Ann. N.Y. Acad. Sci. 501 (1987), 1-13; Pabo, Biochemistry 25 (1986), 5987-5991. The results obtained from the above-described computer analysis can be used in combination with the method of the invention for, e.g., optimizing known inhibitors. Appropriate peptidomimetics and other inhibitors can also be identified by the synthesis of peptidomimetic combinatorial libraries through successive chemical modification and testing the resulting compounds, e.g., according to the methods described herein. Methods for the generation and use of peptidomimetic combinatorial libraries are described in the prior art, for example in Ostresh, Methods in Enzymology 267 (1996), 220-234 and Domer, Bioorg. Med. Chem. 4 (1996), 709-715. Furthermore, the three-dimensional and/or crystallographic structure of inhibitors and the IRF6 protein of the invention can be used for the design of peptidomimetic drugs (Rose, Biochemistry 35 (1996), 12933-12944; Rutenberg, Bioorg. Med. Chem. 4 (1996), 1545-1558).

The invention also relates to pharmaceutical compositions comprising a wild-type IRF6 gene or gene product or the antibodies, and optionally a pharmaceutically acceptable carrier for use in the treatment, amelioration of the symptoms of, or prevention of VWS, PPS, CL/P, or such other cleft lip disorders. The pharmaceutical compositions of this invention can be administered as part of a combinatorial therapy with other agents and treatment regimens.

These pharmaceutical compositions of the invention may conveniently be administered by any of the routes conventionally used for drug administration. Acceptable salts comprise acetate, methylester, HCl, sulfate, chloride and the like. The compounds may be administered in conventional dosage forms prepared by combining the drugs with standard pharmaceutical carriers according to conventional procedures. These procedures may involve mixing, granulating and compressing or dissolving the ingredients as appropriate to the desired preparation. It will be appreciated that the form and character of the pharmaceutically acceptable character or diluent is dictated by the amount of active ingredient with which it is to be combined, the route of administration and other well-known variables. The carrier(s) must be “acceptable” in the sense of being compatible with the other ingredients of the formulation and not deleterious to the recipient thereof. The pharmaceutical carrier employed may be, for example, either a solid or liquid. Exemplary of solid carriers are lactose, terra alba, sucrose, talc, gelatin, agar, pectin, acacia, magnesium stearate, stearic acid, amlyose, starch, dextrose, magnesium stearate, silicic acid, viscous paraffin, perfume oil, fatty acid esters, hydroxxymethylcellulose, polyvinyl pyro-lidone and the like, as well as combinations thereof. Exemplary of liquid carriers are buffered saline solution (e.g., phosphate buffer saline), syrup, oil such as peanut oil and olive oil, water, emulsions, various types of wetting agents, sterile solutions, alcohols, glycerol, ethanol, gum arabic, vegetable oils, benzyl alcohols, polyethylene glycols, and the like, as well as combinations therof. Similarly, the carrier or diluent may include time delay material well known to the art, such as glyceryl mono-stearate or glyceryl distearate alone or with a wax. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active agents.

The pharmaceutical composition, if desired, can also contain minor amounts of wetting or emulsifying agents, or pH buffering agents. The composition can be a liquid solution, suspension, emulsion, tablet, pill, capsule, sustained release formulation, or powder. The pharmaceutical composition can be formulated as a suppository, with traditional binders and carriers such as triglycerides. Oral formulation can include standard carriers such as pharmaceutical grades of mannitol, lactose, starch, magnesium stearate, polyvinyl pyrol-lidone, sodium saccharine, cellulose, magnesium carbonate, etc.

Methods of introduction of these compositions include, but are not limited to, intradermal, intramuscular, intraperitoneal, intraocular, intravenous, subcutaneous, topical, oral and intranasal. Other suitable methods of introduction can also include gene therapy (as described below), rechargeable or biodegradable devices, particle acceleration devises (“gene guns”) and slow release polymeric devices. The pharmaceutical compositions of this invention can also be administered as part of a combinatorial therapy with other agents.

The pharmaceutical composition can be formulated in accordance with the routine procedures as a pharmaceutical composition adapted for administration to human beings. For example, compositions for intravenous administration typically are solutions in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water, saline or dextrose/water. Where the composition is administered by injection, an ampoule of sterile water for injection or saline can be provided so that the ingredients may be mixed prior to administration.

For topical application, nonsprayable forms, viscous to semi-solid or solid forms comprising a carrier compatible with topical application and having a dynamic viscosity preferably greater than water, can be employed. Suitable formulations include but are not limited to solutions, suspensions, emulsions, creams, ointments, powders, enemas, lotions, sols, liniments, salves, aerosols, etc., which are, if desired, sterilized or mixed with auxiliary agents, e.g., preservatives, stabilizers, wetting agents, buffers or salts for influencing osmotic pressure, etc. The agent may be incorporated into a cosmetic formulation. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., pressurized air.

Agents described herein can be formulated as neutral or salt forms.

Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The agents are administered in a therapeutically effective amount. The amount of agents which will be therapeutically effective in the treatment of a particular disorder or condition will depend on the nature of the disorder or condition, and can be determined by standard clinical techniques. In addition, in vitro or in vivo assays may optionally be employed to help identify optimal dosage ranges. The precise dose to be employed in the formulation will also depend on the route of administration, and the seriousness of the IRF6 associated dysfunctions or dysregulations such as VWS, PPS, or CL/P, and should be decided according to the judgment of a practitioner and each patient's circumstances. As is well known in the medical arts, dosages for any one patient depends upon many factors, including the patient's size, body surface area, age, the particular compound to be administered, sex, time and route of administration, general health, and other drugs being administered concurrently. Progress can be monitored by periodic assessment. Effective doses may be extrapolated from dose-response curves derived from in vitro or animal model test systems.

Furthermore, the use of pharmaceutical compositions which comprise antisense-oligonucleotides which specifically hybridize to RNA encoding mutated versions of a IRF6 gene or which comprise antibodies specifically recognizing mutated IRF6 protein but not or not substantially the functional wild-type form is conceivable in cases in which the concentration of the mutated form in the cells should be reduced.

In accordance with the present invention, the particular drug selection, dosage regimen and corresponding patients to be treated can be determined in accordance with the present invention. The dosing recommendations will be indicated in product labeling by allowing the prescriber to anticipate dose adjustments depending on the considered patient group, with information that avoids prescribing the wrong drug to the wrong patients at the wrong dose.

In a further embodiment the invention relates to a method for the production of a pharmaceutical composition comprising the steps of any one of the above described methods and synthesizing and/or formulating the compound identified or a derivative or homologue thereof in a pharmaceutically acceptable form. The therapeutically useful compounds identified according to the method of the invention may be formulated and administered to a patient as discussed above. For uses and therapeutic doses determined to be appropriate by one skilled in the art.

Furthermore, the present invention relates to a method for the preparation of a pharmaceutical composition comprising the steps of the above-described methods; and formulating a drug or pro-drug in the form suitable for therapeutic application and preventing or ameliorating the disorder of the subject diagnosed in the method of the invention. Drugs or pro-drugs after their in vivo administration are metabolized in order to be eliminated either by excretion or by metabolism to one or more active or inactive metabolites (Meyer, J. Pharmacokinet. Biopharm. 24 (1996), 449-459). Thus, rather than using the actual compound or inhibitor identified and obtained in accordance with the methods of the present invention a corresponding formulation as a pro-drug can be used which is converted into its active in the patient. Precautionary measures that may be taken for the application of pro-drugs and drugs are described in the literature; see, for review, Ozama, J. Tocicol Sci. 21 (1996), 323-329).

Furthermore, the present invention contemplates a kit comprising any one of the afore-described polynucleotides, oligonucleotides, probes, vectors, host cells, proteins, antibodies, inhibitors, activators or nucleic acid molecules of the invention, and optionally suitable means for detection.

The kit of the invention may contain further ingredients such as selection markers and components for selective media suitable for the generation of transgenic cells and animals. The kit of the invention may advantageously be used for carrying out a method of the invention and could be, inter alia, employed in a variety of applications, e.g., in the diagnostic field or as research tool. The parts of the kit of the invention can be packaged individually in vials or in combination in containers or multicontainer units. Manufacture of the kit follows preferably standard procedures which are known to the person skilled in the art. The kit or diagnostic compositions may be used for methods for detecting expression of the IRF6 gene in accordance with any one of the above-described methods of the invention, employing, for example, immunoassay techniques such as radioimmunoassay or enzyme-immunoassay or preferably nucleic acid hybridization and/or amplification techniques such as those described herein before and in the examples.

The invention contemplates using methods of gene therapy to deliver a therapeutic nucleic acid to an e.g., cell, animal, or subject that mediates a therapeutic effect. Gene therapy refers to treatment or prevention of a disease performed by the administration of a nucleic acid to a subject. Gene therapy is based on introducing therapeutic genes into cells by ex-vivo or in vivo techniques, is one of the most important applications of gene transfer. Suitable vectors and methods for in vitro or in vivo gene therapy are described in the literature and are known to the person skilled in the art; see, e.g., Giordano, Nature Medicine 2 (1996), 534-539; Schaper, Circ. Res. 79 (1996), 911-919; Anderson, Science 256 (1992), 808-813; Isner, Lancet 348 (1996), 370-374; Muhlhauser, Circ. Res. 77 (1995), 1077-1086; Wang, Nature Medicine 2 (1996), 714-716; WO 94/29469; WO 97/00957 or Schaper, Current Opinion in Biotechnology 7 (1996), 635-640, and references cited therein. The gene may be designed for direct introduction or for introduction via liposomes, or viral vectors (e.g., adenoviral, retroviral) into the cell. Preferably, said cell is a germ line cell, embryonic cell, or egg cell or derived therefrom, most preferably said cell is a stem cell.

For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5):155-215). Methods commonly known in the art of recombinant DNA technology which can be used are described in Ausubel et al. (eds.), 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds.), 1994, Current Protocols in Human Genetics, John Wiley & Sons, NY.

The invention will now be described by reference to the following examples which are merely illustrative and are not to be construed as a limitation of the scope of the present invention.

### EXAMPLES

### Example 1

### Mutations in IRF6

Interferon regulatory factor 6 (IRF6) belongs to a family of nine transcription factors that share a highly conserved helix-turn-helix DNA-binding domain and a less conserved protein-binding domain. Most IRFs regulate the expression of interferon-α (alpha) and -β (beta) after viral infection (Taniguchi et al., 2001), but the function of IRF6 is unknown. The gene encoding IRF6 is located in the critical region for the Van der Woude syndrome (VWS; OMIM 119300) locus at chromosome 1q32-q41 (Murray et al., 1990; Schutte et al. 2000). The disorder is an autosomal dominant form of cleft lip and palate with lip pits (Van der Woude 1954), and is the most common syndromic form of cleft lip or palate. Popliteal pterygium syndrome (PPS; OMIM 119500) is a disorder with a similar orofacial phenotype that also includes skin and genital anomalies (Gorlin et al., 1968). Phenotypic overlap (Bixler et al., 1973) and linkage data (Lees et al., 1999) suggest that these two disorders are allelic. We found a nonsense mutation in IRF6 in the affected twin of a pair of monozygotic twins who were discordant for VWS. Subsequently, we identified mutations in IRF6 in 45 additional unrelated families affected with VWS and distinct mutations in 13 families affected with PPS. Expression analyses showed high levels of IRF6 mRNA along the medial edge of the fusing palate, tooth buds, hair follicles, genitalia and skin. Our observations demonstrate that haploinsufficiency of IRF6 disrupts orofacial development and are consistent with dominant-negative mutations disturbing development of the skin and genitalia.

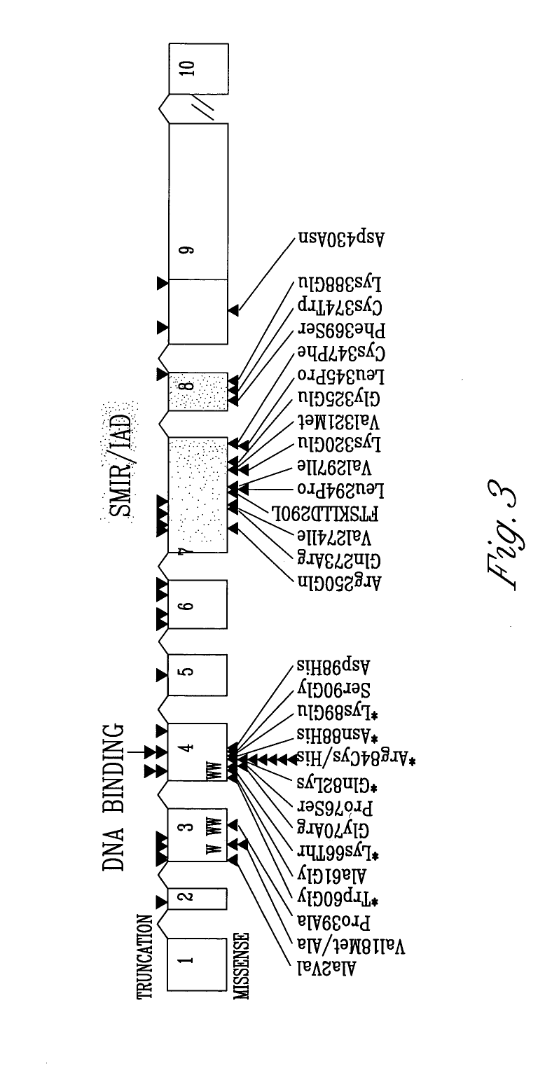

To identify the locus associated with VWS, we carried out direct sequence analysis of genes and presumptive transcripts in the 350-kilobase (kb) critical region (Schutte et al., 2000). This approach is confounded by single-nucleotide polymorphisms (SNPs), normal DNA sequence variation that occurs about once every 1,900 base pairs (Sachidanandam et al., 2001) (bp). To distinguish between putative disease-causing mutations and SNPs, we studied a pair of monozygotic twins discordant for the VWS phenotype and whose parents were unaffected. Monozygotic status was confirmed by showing complete concordance of genotype at 20 microsatellite loci. We proposed that the only sequence difference between the twins would result from a somatic mutation found only in the affected twin. We identified a nonsense mutation in exon 4 of IRF6 in the affected twin, which was absent in both parents and the unaffected twin. We subsequently identified mutations in 45 additional unrelated families affected with VWS and in 13 families affected with PPS (FIG. 3; Table 1), demonstrating unequivocally that these two syndromes are allelic (Bixler et al., 1973; Lees et al., 1999). These mutations were not observed in a minimum of 180 control chromosomes.

Clefts of the lip with or without cleft palate and isolated cleft palate are developmentally and genetically distinct (Fraser, 1955), yet VWS is a single-gene disorder that encompasses both clefting phenotypes. To verify this, we analyzed pedigrees (n=22) that had a single mutation in IRF6 and affected individuals with both phenotypes. Genotype analysis of family VWS25 demonstrated that affected individuals, regardless of their phenotype, shared the 18-bp deletion found in the proband (data not shown). We observed similar results in the other families and conclude that a single mutation in IRF6 can cause both types of cleft.

To determine the effect of mutations on IRF6 gene activity, we compared the type and position of the mutation with the phenotype. Previous identification of deletions encompassing the VWS locus (including IRF6 in its entirety) had suggested that the phenotype is caused by haploinsufficiency (Bocian et al., 1987; Sander et al., 1994; Schutte et al., 1999). In this study, we found protein-truncation (nonsense and frameshift) mutations in 22 families (FIG. 3). Protein-truncation mutations were significantly more common in VWS than in PPS (P=0.004) and were consistent with haploinsufficiency in the VWS pedigrees. The lone exception to this relationship was a nonsense mutation introducing a stop codon in place of a glutamine codon at position 393, found in pedigree PPS11, which may be a dominant-negative mutation (see below).

The position of the missense mutations provides insight into the structure and function of the IRF6 gene product. When we aligned the family of IRF proteins, we observed that IRF6 has two conserved domains (FIG. 3), a winged-helix DNA-binding domain (amino acids 13-113) and a protein-binding domain (amino acids 226-394) termed SMIR (Smad-interferon regulatory factor-binding domain) (Eroshkin et al., 1999). Studies of IRF3 and IRF7 have shown that the SMIR domain is required to form homo- and heterodimers (Mamane et al., 1999; Au et al., 2001). The dimers then translocate to the nucleus, associate with other transcription factors and ultimately bind to their DNA targets (Mamane et al., 1999). Of the missense mutations, 35 of 37 localized to regions encoding these two domains. This distribution is non-random (P<0.001), and we conclude that the domains are critical for IRF6 function.

Whereas the missense mutations that cause VWS were almost evenly divided between the two domains, most missense mutations that cause PPS were found in the DNA-binding domain (11 of 13, FIG. 3). This distribution is significant (P=0.03) and suggests that missense mutations in the DNA-binding domain associated with VWS and PPS affect IRF6 function differently. When we compared their positions with the crystal structure of the IRF1 DNA-binding domain (Escalante et al., 1998), we found that every amino-acid residue that was mutant in individuals with PPS directly contacts the DNA, whereas only one of seven of the residues mutant in the individuals with VWS contacts the DNA. Most notably, we observed missense mutations involving the same residue, Arg84, in seven unrelated PPS families. The Arg84 residue is comparable to the Arg82 residue of IRF1. It is one of four residues that make critical contacts with the core sequence, GAAA, and is essential for DNA binding (Escalante et al., 1998). The observed change of this residue to a cysteine or histidine caused a complete loss of that essential contact. One possible explanation for this apparent genotype-phenotype relationship is that missense mutations that cause VWS are due to a complete loss of function of the mutated IRF6 protein, affecting both DNA and protein binding, whereas missense mutations causing PPS affect only IRF6's ability to bind DNA. The ability of the mutated IRF6 to bind to other proteins is unaffected, and it therefore forms inactive transcription complexes; thus, this is a dominant-negative mutation. Similarly, deletion of the DNA-binding domain of IRF3 or IRF7 exerts a dominant-negative effect on the virus-induced expression of the type I interferon genes and the RANTES gene (Au et al., 2001; Lin et al., 1999).

To correlate the expression of IRF6 with the phenotypes of VWS and PPS, we carried out RT-PCR, northern-blot analysis and whole-mount in situ hybridization. We found that IRF6 was broadly expressed in embryonic and adult mouse tissues (data not shown), a pattern also seen in human fetal and adult tissues (data not shown). Greater expression of IRF6 seemed to occur in secondary palates dissected from day 14.5-15 mouse embryos and in adult skin. Whole-mount in situ hybridization demonstrated that IRF6 transcripts were highly expressed in the medial edges of the paired palatal shelves immediately before, and during, their fusion. Similarly high IRF6 expression was seen in the hair follicles and palatal rugae, tooth germs and thyroglossal duct and external genitalia, and in skin throughout the body (data not shown). These observations are in accord with the VWS/PPS phenotype: notably, 20% of individuals with VWS exhibit agenesis of the second premolar teeth and 40% of individuals with PPS display genital anomalies.

Although we demonstrated that VWS and PPS are caused by mutations in a single gene, the phenotype for any given mutation varied in at least three ways even within the same family. Of the families with known mutations, we observed 32 families with multiple combinations of orofacial anomalies, 22 families with mixed clefting phenotypes (individuals with cleft lip and individuals with cleft palate only in the same family) and four families affected with PPS that included individuals who exhibit orofacial (VWS) features exclusively. The marked phenotypic variation in our cohort strongly implicates the action of stochastic factors or modifier genes on IRF6 function. In this context, we identified the sequence variant Val274Ile (FIG. 3). This variant occurs at an absolutely conserved residue within the SMIR domain, is common in unaffected populations (3% in European-descended and 22% in Asian populations) and is an attractive candidate for a modifier of VWS, PPS, and other orofusial clefting disorders.

The mixed clefting phenotype is common in families affected with VWS, but very rare in families with nonsyndromic orofacial clefts, and is not seen in most other syndromic forms of orofacial clefts. It is, however, also seen in clefting disorders caused by mutations in the genes MSXI (van den Boogaard et al., 2000) and TP63 (Celli et al., 1999; McGrath et al., 2001), suggesting that these may be involved in a common genetic pathway. In support of a common pathway, we found two IRF binding sites in the promoter MSX1 and one in the intron, all of which are conserved between human and mouse.

We are taking an integrated approach to dissecting the complex pathways that underlie development of the lip and palate, including genetic analysis to identify the mutations that cause orofacial clefts. The discordant monozygotic twins proved useful in this effort, and provided proof of principle (Machin, 1996) that discordant monozygotic pairs can be used to search for modifiers or mutations, especially in regard to complex traits where mapping may be imprecise and mutation analysis may be confounded by SNPs. We also used a large number of samples from unrelated individuals to confirm that mutations in IRF6 are pathogenic for both VWS and PPS and to prove that IRF6 is essential for development of the lip and palate and is involved in development of the skin and external genitalia. The SMIR domain has been proposed to mediate an interaction between IRFs and Smads (Eroshkin et al., 1999), a family of transcription factors known to transduce TGF-β signals (Brivanlou et al., 2002). In addition, the expression of Irf6 along the medial edge of the palate seems to overlap with Tgfb3 (Fitzpatrick et al., 1990), and Tgfb3, along with other members of this superfamily such as Tgfb2 and Inhba, is required for palatal fusion (Proetzel et al., 1995; Sanford et al., 1997; Matzuk et al., 1995; Kaartinen et al., 1995). Together with our data, these observations support a role for IRF6 in the transforming growth factor-β (TGF-β) signaling pathway, a developmental pathway of fundamental significance. The identification of IRF6 as a key determinant in orofacial development will help us to further delineate and integrate the molecular pathways underlying morphogenesis of the lip and palate.

Methods

Families. Families affected with VWS (n=107) and PPS (n=15) were identified and examined by one or more geneticists or clinicians as previously described (Schutte et al. 1999). Nearly all families are of northern European descent. Sample collection and inclusion criteria for VWS and PPS were described previously (Taniguchi et al., 2001). We obtained written informed consent from all subjects and approval for all protocols from the Institutional Review Boards at the University of Iowa and at the University of Manchester.

Mutation analysis. We amplified exons 1-8 and part of axons 9 and 10 by standard PCR. The amplified products were purified (Qiagen) and directly sequenced with an ABI Prism 3700. The sequence was analyzed using the computer program PolyPhred.

Protein modeling. The IRF6 protein structure was predicted from its amino-acid sequence using Expasy, and aligned with the known crystalline structure of the DNA-binding domain of IRF1 using the UNIX-based computer software package Quanta (Accelrys). To model the mutations found at position Arg84 in the IRF6 DNA-binding domain, the residue was manually altered to a cysteine or a histidine. The package predicts all possible orientations of the altered side chain and displays the position with the highest probability.

RT-PCR. We extracted total RNA using a standard guanidinium isothiocyanate, acid-phenol protocol. RT-PCR analyses were performed and analyzed as detailed previously (Dixon et al., 1997) using a forward primer designed in exon 4 and a reverse primer designed in axon 6 of IRF6. These primers generate a single product of 212 bp from cDNA.

Northern-blot analysis. A multiple-tissue northern blot (Seegene) was hybridized with a probe generated by PCR using primers derived from the distal end of the 3′ untranslated region of IRF6 and labeled as recommended by the manufacturer with the StripE-Z system (Ambion). We hybridized the blot in Express Hyb (Clontech), washed it as recommended and exposed it to X-ray film for 72 h at −80° C.

Whole-mount in situ hybridization. Sense and anti-sense riboprobes were 1,600 bp in length, derived from the 3′ untranslated region of IRF6 and generated with Sp6 and T7 promoters, respectively. We fixed embryos dissected from time-mated MF1 mice in 4% paraformaldehyde overnight, processed them and subjected them to hybridization with sense or antisense probes as described previously (Nieto et al., 1996).

Statistical analysis. Statistical significance of mutation location was calculated with the Fisher's exact test using the assumption of equal probability for a mutation at each residue.

### Example 2

### IRF6 is a Major Gene for Isolated Cleft Lip and Palate

Common human disorders, including the most frequently occurring birth defects, have complex etiologies. These traits arise from the interplay of multiple genetic and environmental factors, making identification of contributing components difficult. Complex traits are characterized by familial aggregation but recurrence rates that are modest, typically less than 5% for a sibling to also be affected. Cleft lip and/or palate (CL/P) is a common birth defect with prevalence varying according to geographic origin, with Asian and Amer-Indian populations having the highest rates and African-derived groups the lowest (Mossey et al., 2002). Isolated CL/P comprises about 70% of all disorders with a cleft, with the remaining 30% being divided across several hundred Mendelian, chromosomal, teratogenic, and sporadic conditions that typically include other birth defects.

There is a significant challenge in identifying the genetic and environmental causes of complex traits as the numbers of genes and environmental triggers are predicted to be large. Genetic factors can be investigated using family studies to localize genes based on linkage approaches. This strategy requires the collection of hundreds of families with two or more affected members. Obtaining DNA samples from this many individuals is difficult for a birth defect where recurrence risks are low. Thus, to date there has been limited success in confirming contributions to isolated CL/P using linkage analysis.

A complementary strategy is to use candidate genes selected for the role they play in the development of the disorder of interest. For isolated CL/P, this includes genes that are expressed at the time of lip or palate closure in the embryo. A second selection strategy is to choose representatives from single gene Mendelian disorders whose clinical expression closely approximates that of the complex trait. For isolated CL/P, perhaps the best example is the Van der Woude syndrome (VWS), an autosomal dominant disorder in which lower lip pits is the only feature distinguishing VWS from isolated CL/P (Burdick et al., 1985).

We recently reported that mutations in the Interferon Regulatory Factor 6 gene (IRF6) cause VWS. In searching the gene for mutations, we also identified a common polymorphic variant, V274I, in the protein-binding domain. Since the valine found at this site is strongly conserved in IRF6 across species, we hypothesized that this variant might affect gene function and contribute to CL/P. This study reports our evaluation of V274I and other polymorphisms in IRF6 for cases of cleft lip and palate.

Methods

Sample Collections

A summary of the samples used in this study is given in Table 2. Seven of these populations have been described at least in part previously (Marazita et al., Am J Hum Genet (2002); Marazita et al., Cleft Palate Craniofac J (2002); Schultz et al., 2003; Moreno et al., 2003; Shi et al., 2003; Romitti et al., 1999; Field et al., 1994; Vieira et al., 2003). Populations from Japan, Vietnam and Brazil were collected as part of investigations of genetic and/or environmental causes of clefting. All individuals were screened for the presence of associated anomalies/syndromes and only cleft cases determined to be isolated were used in this study. All populations had institutional approval for participation and signed informed consent was obtained in all cases. For most samples, whole blood was collected and processed using QiaAmp Blood kits (Qiagen). When blood collection was not possible, DNA was purified from buccal swabs.

Mothers in the Philippines (184), all of whose children were unaffected with CL/P, were used as controls for the Philippines studies. Unrelated controls were also available for case-control comparison in the Indian (84 cases and 40 controls) and Chinese (160 cases and 24 controls) studies. A collection of 1,064 DNA samples representing 51 different ancestral populations (the “CEPH Diversity Panel”) was obtained from the Centre d'Étude du Polymorphisme Humain (CEPH) for the purpose of observing worldwide allele frequencies (Cann et al., 2002).

TABLE 2
Summary of all samples analyzed in association tests and
control samples. A breakdown of the cleft phenotypes for
the individuals studied in each case population given
by (CL = cleft lip alone; CLP = cleft lip with cleft
palate; CP = cleft palate alone; UNK = unknown
cleft type).

| | | | | Family | Mean | | | | |
|---|---|---|---|---|---|---|---|---|---|
| | Region of | | No. | Size | Family | | | | |
| Subset | Origin | Birthplace | Families | Range | Size | CL | CLP | CP | UNK |
| 1 | East Asia | Japan | 115 | 3-4 | 3.0 +/− 0.1 | 26 | 73 | 12 | 0 |
| | | Vietnam | 175 | 3-4 | 3.0 +/− 0.1 | 50 | 109 | 16 | 0 |
| | | China | 97 | 3-26 | 10.6 +/− 4.7 | 33 | 64 | 0 | 0 |
| | | Philippines | 403 | 3-76 | 15.1 +/− 15.2 | 116 | 282 | 5 | 0 |
| 2 | South | Brazil | 303 | 3 | 3 | 88 | 161 | 51 | 3 |
| | America | | | | | | | | |
| | | Columbia | 107 | 3-19 | 6.7 +/− 4.4 | 17 | 85 | 0 | 5 |
| | | ECLAMC | 233 | 2 | 2 | 44 | 98 | 16 | 0 |
| 3 | Europe | Denmark | 239 | 3-8 | 4.3 +/− 0.9 | 81 | 105 | 53 | 0 |
| | | Iowa | 246 | 3 | 3 | 69 | 109 | 68 | 0 |
| 4 | India | India | 50 | 4-38 | 14.2 +/− 6.5 | 36 | 14 | 0 | 0 |
| Controls | CEPH | Africa | 156 | 1 | 1 | | | | |
| | Diversity | | | | | | | | |
| | Panel | | | | | | | | |
| | | Pakistan | 200 | 1 | 1 | | | | |
| | | China | 183 | 1 | 1 | | | | |
| | | South | 108 | 1 | 1 | | | | |
| | | America* | | | | | | | |
| | East Asia | Philippines | 238 | 1 | 1 | | | | |
| | | China | 24 | 1 | 1 | | | | |
| | India | India | 40 | 1 | 1 | | | | |

*Individual are from Native American ancestral populations

Genotyping

Genotyping for single nucleotide polymorphisms (SNPs) was performed using one of two protocols on an ABI PRISM® 7900HT Sequence Detection System. The V2741 polymorphism was genotyped with an allele-specific kinetic PCR reaction (Shi et al., 2003). Genotyping for all other markers was performed using the TagMan® chemistry (Applied Biosystems). Additional SNPs within the IRF6 genomic region were found through sequencing of the gene, while other SNPs in the region surrounding IRF6 were chosen based on location using the Celera Discovery System (worldwideweb.celeradiscoverysystem.com). SNP Genotyping Assays were obtained from Applied Biosystems either through the Assay-On-Demand™ service for six previously identified polymorphisms, or through the Assay-By-Design™ service by providing sequences to the company for the design of the additional 29 assays. Reactions were carried out using standard conditions supplied by the company and performed in duplicate to confirm the results. Genotyping for the family studies was carried out at the Center for Inherited Disease Research (CIDR) using the Weber Screening Set 9 of microsatellite repeat markers (worldwideweb.cidr.jhmi.edu).

Sequencing

Sequencing of the 23 kb IRF6 genomic region was conducted on 24 individuals: 20 Filipino CL/P cases that carry two valine (high risk) alleles at the V2741 site, two Filipino CL/P cases that carry two isoleucine (low risk) alleles at the V2741 site, and two controls of European descent. Sequencing of the coding portion of the exons was also performed on 160 individuals with isolated CL/P. In addition, three regions of homology to the corresponding region of mouse sequence of 200-300 bps each (homology defined as >75% homology over at least 100 bp), located approximately 81 Kb, 103 Kb and 117 Kb 3′ of IRF6, were sequenced in a panel of CL/P affected individuals from the Philippines (113) and Iowa (93), 140 control individuals from the Philippines and 96 CEPH samples. All sequencing reactions were performed using ABI PRISM® Big Dye™ Terminator sequencing chemistry from Applied Biosystems.

Statistical Analysis

Preliminary Analyses. The inheritance of each IRF6 marker in families was analyzed with PedCheck (O'Connell et al., 1998) to test for inconsistencies due to non-paternity or other errors. For linkage analyses, allele frequencies were estimated from the unaffected founders in the study families. The genetic model parameters were taken from segregation analysis results for the Chinese families (Marazita et al, 1992), for the Indian families (Ray et al., 1993), and for the Filipino families (unpublished results).

Tests of Hardy-Weinberg equilibrium and case-control comparisons. Chi-square tests were used to assess Hardy-Weinberg equilibrium of V2741 in the founders and unrelated individuals from the families in each study population due to the possibility of false positives in two allele systems (Schaid et al., 1999). Chi-square statistics were also used to perform case-control comparisons.

Linkage Calculations. Of the ten populations genotyped for V2741, four included extended pedigrees and were therefore appropriate for parametric linkage analyses: Colombian, Chinese, Indian and Filipino. Three of these study populations (Chinese, Indian and Filipino) had the Weber Screening Set 9 markers genotyped on chromosome 1 as well as V2741 and therefore were appropriate for multipoint calculations. We calculated two-point LOD scores in the extended kindreds using the Elston-Stewart algorithm (Elston et al., 1971), employing the LINKAGE program with recent updates to speed calculations (Cottingham et al., 1999; O'Connell et al., 1995; Terwilliger et al., 1994). We calculated multipoint LOD score statistics using the descent graph approach implemented in SIMWALK2 (Sobel et al., 1996) and multipoint NPL statistics using MERLIN (Abecasis et al., 2002).

Allelic Association: Transmission Disequilibrium Test (TDT) Method. Alleles at each IRF6 marker were tested for association with CL/P using the Family Based Association Test (Horvath et al., 2001; Laird et al., 2000; Rabinowitz et al., 2000). In the ECLAMC data, only mother-child pairs were available; for those TDT calculations, we applied the likelihood ratio test (LRT) of Weinberg (Weinberg et al., 1999) under the assumption that the distribution of paternal alleles is the same as the maternal.

TDT was also utilized to assess control transmission distortion, i.e. transmission to either unaffected siblings in proband nuclear families (Philippines) or to control children in control triads (Iowa), to investigate the possibility of biased overtransmission (Mitchell et al., 2003). Finally, because a number of the studies included large extended kindreds, all TDT analyses were repeated including only the proband nuclear families from each of the extended kindreds.

In order to derive a summary association statistic across populations for V2741, a random-effects meta-analysis model, as described by Dersimonian and Laird (DerSimonian et al., 1986), was applied to estimate the odds ratio of the associated allele within the proband nuclear families. Before pooling, we estimated Cochran's Q-statistic. There was no significant evidence of heterogeneity overall (Q=9.545, P-value=0.30), nor in the population subsets. A random-effects model was used because it includes both within and between-study components of variance. Because it generally will yield a wider confidence interval, the random effects model is more conservative than a fixed effects model (Berlin et al., 1989).

SNP and Haplotype association analyses. Thirty-six SNPs (nine within IRF6, ten within 100 Kb 5′ of IRF6, and 17 within 200 Kb 3′ of the gene) were studied in 296 parent/case triads from the Philippines. Genotype data were available for a subset of seven of these SNPs in IRF6 for 108 triads from Iowa and 184 triads from Denmark. Individual association values were calculated using FBAT for each of the SNP markers typed on the Filipino and European populations. Haplotype-based transmission disequilibrium statistics were also calculated using the haplotype version of FBAT (Rabinowitz et al., 2000; Access to FBAT and HaploFBATat http://worldwideweb.biostat.harvard.edu/-fbat/fbat.htm). The EM algorithm was used to form the haplotypes and estimate haplotype frequencies, then transmission distortion was assessed.

Attributable Risk: We estimated the attributable risk (AR) for the associated IRF6 allele, i.e. the proportion of CL/P cases in a population that can be attributed to the V allele. AR is a function of the Relative Risk (RR) and the probability of exposure given disease (P[E/D}). We estimated AR as P[E/D](RR-1)/RR, using the OR as the estimator for RR.

Results

Gene Structure and SNP Localization

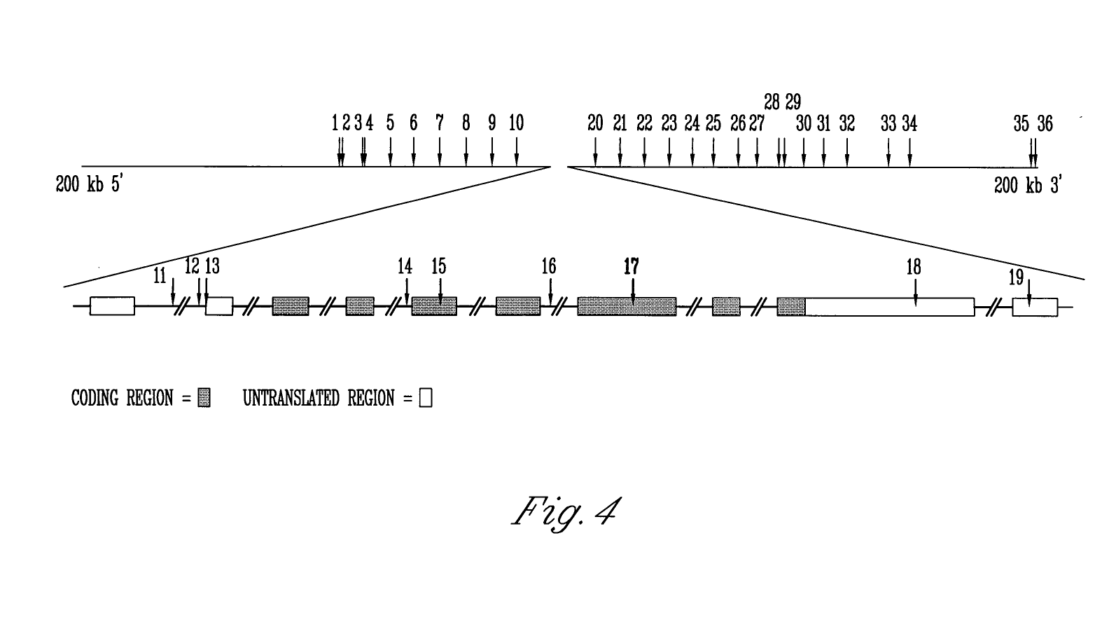

The structure of the IRF6 gene with its intron/exon boundaries, flanking regions and the location of each SNP analyzed are shown in FIG. 4.

Allele Frequencies and Case-Control Comparisons

Only the genotypes derived from the population in Brazil showed a significant deviation from Hardy-Weinberg equilibrium (P<0.001). Brazilian populations have a high degree of admixture from groups of different ancestral origins (Native South American, African and European) that may account for these results (Vieira et al., 2003). There was a positive association with V2741 only in the Indian case-control comparison (X²=4.25, P-value=0.039). The CEPH Diversity Panel was also genotyped for the V2741 variant (results not shown). No Africans in this panel carried the I allele and the V allele is present in Pan troglodytes, indicating that the V allele is most likely the ancestral allele in Homo sapiens.

Linkage Analysis Results

The two-point LOD scores with IRF6 were negative or weakly positive in each of the populations at each value of the recombination fraction. The highest LOD was 0.636 (recombination fraction=0.20) in the Indian families. The Indian families also had a statistically significant NPL result (P=0.004), and the Philippines families had a borderline positive NPL result (P=0.06). The multipoint LOD scores for the chromosome 1 microsatellite markers plus V2741 were uniformly negative, while the multipoint HLOD values were weakly positive (the highest multipoint HLOD at IRF6 was 0.382 in the Chinese families). Interestingly, a recent meta-analysis of all CL/P genome scans (including the Filipino, Chinese, Indian, and Colombian families reported here) found significant evidence in favor of linkage to the region containing IRF6 (P-value=0.02) (Marazita et al., 2003).

TDT Association Results

All TDT calculations were done for subdivisions of the data based on proband affection status (CL=cleft lip alone, CL/P=cleft lip plus cleft palate, CP=cleft palate alone, ANY LIP=CL+CL/P).

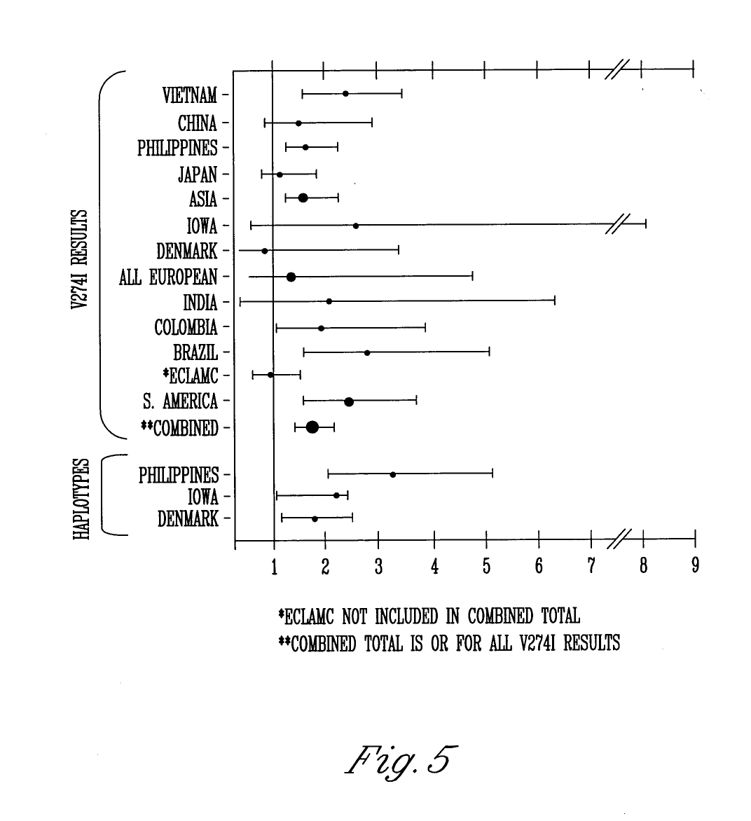

The data was also separated into four subgroups for analysis based on ancestral origin as delineated in Table 2: Asian, South American, European and Indian. In the South American and Asian subgroups, the results were highly significant (P-values<0.001) for association with the common allele (V) in all subdivisions of the data by affection status except for CID alone. In the Indian and European group, there was a nonsignificant trend towards positive association with the common allele (except for the CP subgroup). The V2741 marker had low heterozygosity in the Indian and European populations. When only the proband nuclear families from each of the extended kindreds are included in the TDT analysis, the same pattern of results was seen with significant over transmission of the V allele in all subgroups except for families in which the proband had CP alone (results not shown). The LRT analyses in the ECLAMC population showed no evidence of transmission distortion with V2741. FIG. 5 summarizes the odds ratios estimated in the proband nuclear families in each population and population subgroup.

There were no parent-specific differences in the patterns of association (results not shown). When the TDT was carried out on control (unaffected sibs from the same families) samples there was no significant transmission distortion seen (results not shown), except in the Chinese population where there was a slight association with the less common allele (i.e., no evidence of an overall bias towards the common allele).

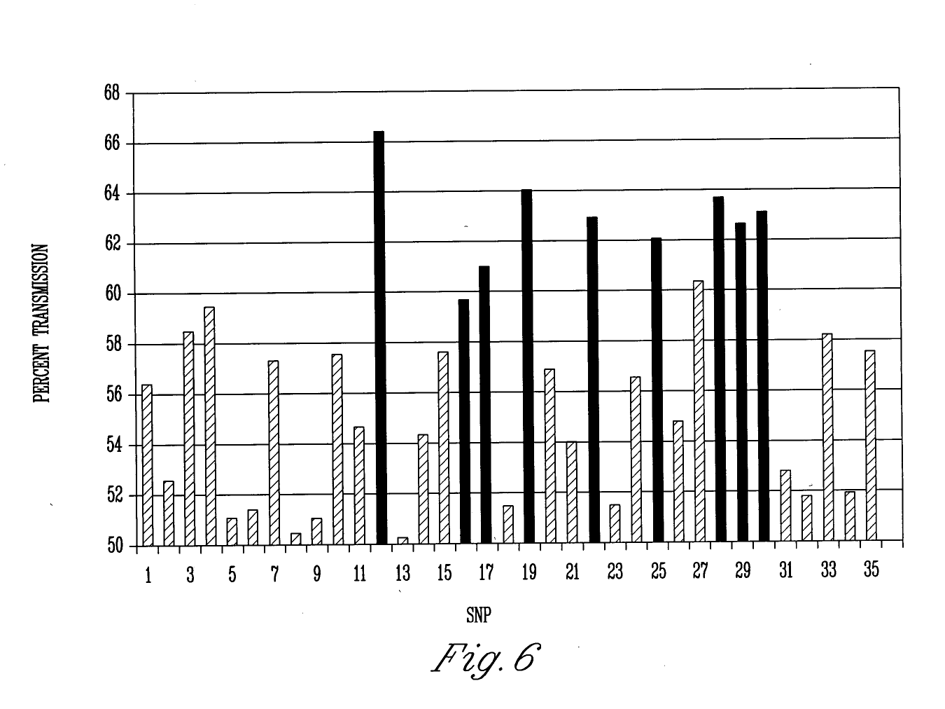

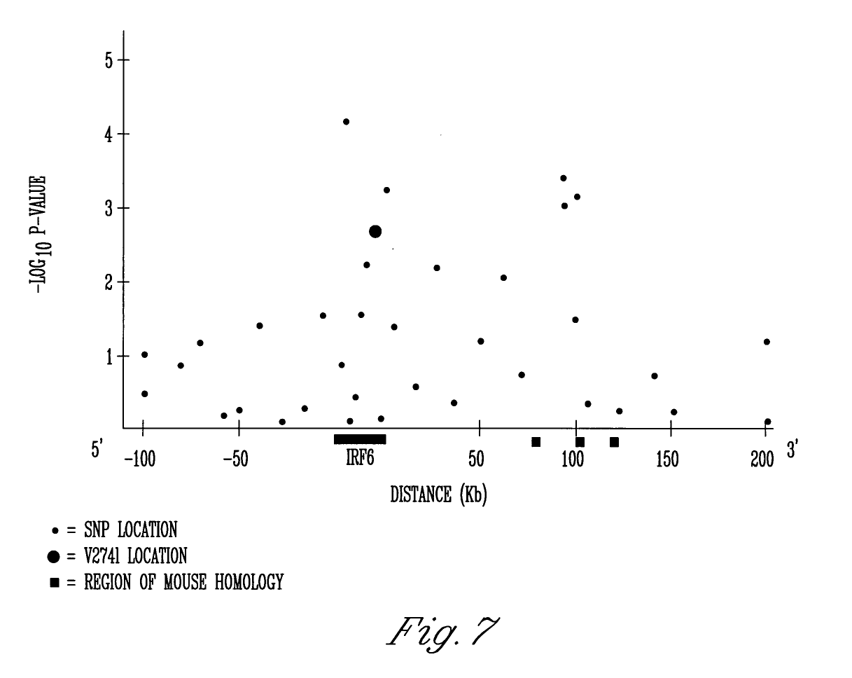

TDT analysis and calculations of percent overtransmission were performed on eight additional SNPs within the IRF6 gene, 10 SNPs that flank IRF6 at a distance of up to 80 Kb in the 5′ direction and 17 SNPs at various distances from 10 to 200 Kb in the 3′ direction (see FIG. 4). Nine of these markers, including V2741 (SNP17) (SNPs 12, 16, 17, 19, 22, 25, 27, 28 and 29), were significantly associated with clefting (P-value<0.01). FIG. 6 summarizes the results by showing the percent overtransmission above the expected 50% for an allele of each of the 36 SNPs analyzed. FIG. 7 shows a graph of the P-value obtained from the TDT analysis for each of the 36 SNPs, graphed according to physical location.

Haplotype Transmission Results

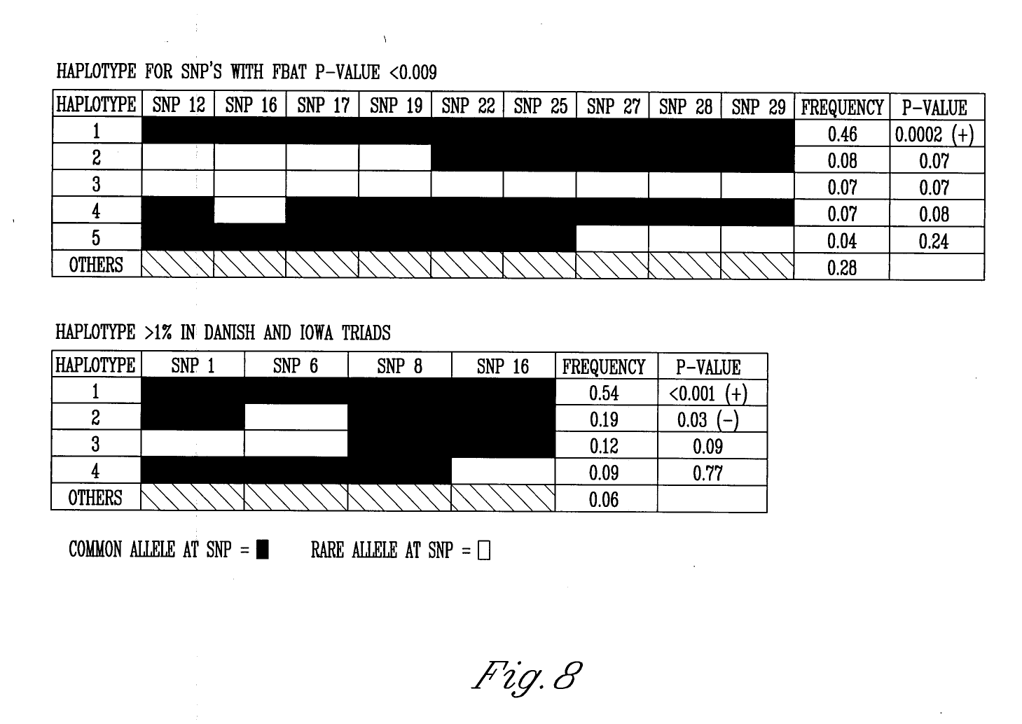

FIG. 8 summarizes the results for all haplotypes with a frequency of >1% in either the Filipinos and Europeans. Data on the Filipinos was obtained in 296 triads and haplotypes are shown for nine SNPs that defined the haplotypes. The haplotype consisting of all the common alleles at each SNP (estimated haplotype frequency=46%) had the most significant transmission distortion (P-value=0.0002). The European haplotype results, obtained in 108 Iowa triads and 184 Danish triads, are shown using the four SNPs that defined these haplotypes. The haplotype consisting of the common allele at each SNP (frequency 53.8% in Iowa, 54.0% Danish) was significantly associated with clefting (P-value=0.038 in Iowans; 0.006 in Danish). FIG. 5 shows the ORs for the associated haplotypes in the samples from the Philippines, Iowa and Denmark. Linkage disequilibrium was assessed among all the markers and was presented graphically and in numeric form (not shown). These results suggest there is a long block of linkage disequilibrium extending from about 40 Kb 5′ of IRF6 to at least 100 Kb 3′ of the gene. This is consistent with a block of linkage disequilibrium seen around IRF6 in European samples used to construct the HapMap data (worldwideweb.hapmap.org).

For the comparison of the Filipino cases to population-based controls, the estimated attributable risk (AR) was 11.6%. This AR assumes that the risk factor is causal and uncorrelated to other risk factors, so should be interpreted cautiously.

To further characterize the impact of the associated allele (V) for V2741, we performed a genotype TDT analysis using FBAT (in the entire dataset). The VN homozygote was significantly associated with clefting (P<0.001), while the V/I and I/I genotypes were negatively associated (P<0.005). This pattern is consistent with recessive action of the associated allele. This was further confirmed by the observation that there is a significant difference in the genotype frequency distributions of probands versus unaffected individuals (P<0.001), with the VN frequency increased in probands versus unaffected, and the V/I and I/I frequencies reduced.

Therefore, we investigated sibling recurrence risk by parental IRF6 mating type, utilizing nuclear families from the entire dataset that had one or more affected children and for which both parents were genotyped for IRF6 V2741 (n=1,493 nuclear families). We divided the mating types into two groups—matings that could produce an IRF6 VN homozygote (VV-VV, VV-VI, VI-VI, N=1316 families) versus those that could not (VI-I I, II-11, VV-II, N=177 families). Within each group, we calculated the proportion of families with one affected child versus those with >1 affected child. Because this is not a population-based sample, these are not true empiric RR estimates, but the results were intriguing and may be generalized to families with a positive family history for clefting. For the first group, the recurrence proportion was 9.0% while it was 5.1% in the second group. Although this difference was not statistically significant (P=0.08), the increase is intriguing and more than three times the 2.4% population-based empiric RR estimate for CL/P in the Philippines (Murray et al., 1997).

Sequencing Results

The ten exons of IRF6 sequenced in 160 individuals with isolated CL/P had no missense, nonsense or frameshift mutations, suggesting that individuals with point mutations in IRF6 are rarely misidentified as having isolated CL/P rather than VWS.

Sequencing of the 23 Kb including the entire known intron-exon structure of IRF6 on 24 individuals revealed 58 variants (sequencing results available upon request). The sequence results gave no indications that any particular variant is etiologic. Sequencing of three regions of mouse homology 3′ of IRF6 did not reveal any changes within the most conserved regions.

Discussion

Identification of genetic loci in complex traits is a major challenge when multiple genetic and environmental factors confound single cause analyses. While linkage analysis of large family datasets can distinguish genetic factors of fairly small effect, some genes may be difficult to identify even using large family collections or case control analysis (Pritchard et al., 2002; Page et al., 2003).

One alternative to genome wide linkage or case/control analyses is to use the Transmission Disequilibrium Test (TDT), which determines significance by measuring the distortion from the expected 50:50 transmission of a heterozygous variant from parent to child. When this ratio is significantly shifted, it is evidence that the overtransmitted allele is either linked with or is itself a causal variant. Candidate genes for a TDT study may be selected based on their embryonic expression, their identified role in animal models such as mouse knockouts, or from an overlap in phenotype between a Mendelian disorder and the complex trait.

One previous success in identifying a gene contributing to isolated CL/P built on finding a nonsense mutation in the MSX1 gene (van den Boogaard et al., 2000) in a large family that segregated in individuals with CL/P. Extending this finding, we demonstrated that approximately 2% of CL/P patients have missense mutations in the coding sequence or in highly conserved regulatory elements of MSX1 (Jezewski et al., 2003). MSX1 was selected based on the mouse knockout phenotype that included cleft palate (Satokata et al., 1994) and was supported by association and TDT data (Lidral et al., 1998; Fallin et al., 2003). There are at least three additional genes that cause syndromic forms of CL/P where occasional affected individuals may have expression limited to CL/P. These include TBX22 in X linked clefting and ankyloglossia (Stanier et al., 1993), P63 in EEC syndrome (Celli et al., 1999) and FGFR1 in Kallmanns Syndrome (Dode et al., 2003). However, these recognized exceptions likely contribute to less than 10% of all isolated cases of CL/P. To search for a more significant gene contribution to CL/P, we undertook a study of IRF6, the gene recently shown to carry mutations in patients with VWS.

VWS has a phenotype that directly overlaps with isolated CL/P in that the clefts are typical and only accompanied by lip pits in approximately 85% of VWS cases (Burdick et al., 1985; Kondo et al., 2002). Thus, 15% of VWS cases may present as indistinguishable from isolated CL/P. Missense or regulatory mutations in IRF6 present in the normal population provide a resource of variants that might serve a functional role in contributing to the occurrence or severity of the CL/P phenotype. This functional role can be supported by statistical evidence of transmission distortion of a risk allele.

Our study of ten populations (1,968 total kindreds) with isolated CL/P showed highly significant transmission disequilibrium for the V274I variant in the IRF6 gene. Two possible sources of bias can lead to apparent overtransmission of an allele in TDT analyses of two-allele markers: departures from Hardy-Weinberg equilibrium's (Schaid et al., 1999) and genotyping errors (Mitchell et al., 2003). Of the ten populations investigated, only genotypes of the cases from Brazil showed any significant departure from Hardy-Weinberg equilibrium, which may be explained by the relatively recent admixture within that population. If there had been significant genotyping errors, then overall transmission distortion would be expected, which was excluded by the normal transmission seen in the analysis of the unaffected children.

SNPs within and flanking the IRF6 gene were used to extend the association with IRF6 to determine if the V allele at position 274 had the maximum effect, or whether other variants in linkage disequilibrium with the V allele might be etiologic. Alleles at multiple other SNPs showed significant transmission distortion. The most significant P-value and the greatest degree of overtransmission was observed for a variant located in the first intron four base pairs from the splice site within a noncoding exon that is absent in rodents. Whether this variant itself, or another in association with it, is of functional importance is not yet known.

Additional support that the V allele itself is not causal comes from the strong disequilibrium seen between particular IRF6 haplotypes and CL/P in the European populations, in which the 2741 variant allele is rare. This suggests that the V274 allele is not causal, or that it may share causality with variants at other sites within or near IRF6 that show stronger transmission distortion than the V274 allele does. The linkage disequilibrium extends from 40 Kb 5′ to 100 Kb 3′ of IRF6, consistent with an earlier report showing LD at D1S205, which lies ˜135 Kb 5′ of IRF6 (Houdayer et al., 2001). It is possible that more than one variant might contribute to this effect and that these variants may be different (or in different proportions) in the several ancestral populations we studied in this report. In addition, a combination of variants within a risk haplotype may be required for a case to exhibit biologic effect (CL/P).

A recent meta-analysis of all CL/P genome scans, including the Filipino, Chinese, Indian, and Colombian families reported here, found significant evidence of linkage to the region containing IRF6 even though none of the studies individually showed evidence of linkage (Marazita et al., 2003). This suggests that even large family studies may fail to find evidence for a genetic effect which may only be detected by candidate gene analysis and linkage disequilibrium. Similarly, this effect was found only with TDT analysis and not with case/control data, although there was a borderline trend towards significance with case/control data is some populations.

Recurrence risk analysis suggests that 3 to 6 major genetic loci may contribute to clefting (Schliekelman et al., 2002). For IRF6, we show that there is an attributable risk for CL/P of about 12%, suggesting that it plays a substantial role in the etiology of CL/P. The possible impact on genetic counseling is suggested by a recurrence risk for siblings of 9% in families with a history of CL/P and a child who could have inherited the common risk allele (88% of the families in this study). This is a more than threefold increase compared to the 2.4% recurrence seen in a population based study for CL/P in the Philippines (Murray et al., 1997). If these results are confirmed, IRF6 genotypes could be used to refine recurrence estimates in genetic counseling of this common disorder. Two genes, MSX1 and IRF6, now seem to play a measurable role in CL/P.

We have demonstrated that informed candidate gene selection can identify specific variants playing a role in complex traits that may be missed by genome wide linkage scans. Direct identification of genes can improve genetic counseling, assist in the identification of new genetic and environmental causes and provide treatment options when correlated with treatment efficacy.

References

Access to FBAT and HaploFBATat http://www.biostat.harvard.edu/-fbat/fbat.htm.
Au, W. C., Yeow, W. S. & Pitha, P. M. 2001. Analysis of functional domains of interferon regulatory factor 7 and its association with IRF-3. Virology 280, 273-282.
Abecasis G R, Cherny S S, Cookson W O, Cardon L R. 2002. Merlin—rapid analysis of dense genetic maps using sparse gene flow trees. Nat Genet 30:97-101.
Berlin J A, Laird N M, Sacks H S, Chalmers T C. 1989. A comparison of statistical methods for combining event rates from clinical trials. Stat Med 8:141-51.
Bixler, D., Poland, C. & Nonce, W. E. 1973. Phenotypic variation in the Popliteal pterygium syndrome. Clin. Genet. 4, 220-228.
Bocian, M. & Walker, A. P. 1987. Lip pits and deletion 1q32-q41. Am. J. Med. Genet. 26: 437-443.
Brivanlou, A. H. & Darnell, J. E., Jr. 2002. Signal transduction and the control of gene expression. Science 295,813-818.
Burdick A B, Bixler D and Puckett C L. 1985. Genetic Analysis in Families With van der Woude Syndrome. J Craniofac Genet Dev Biol. 5:181-208.
Cann H M, de Toma C, Cazes L, et al. 2002. A human genome diversity cell line panel. Science 296:261-2.
Celli, J. et al. 1999. Heterozygous germline mutations in the p53 homolog p63 are the cause of EEC syndrome. Cell 99, 143-153.
Cottingham R W, Jr., Idury R M, Schaffer A A. 1993. Faster sequential genetic linkage computations. Am J Hum Genet 53:252-63.
DerSimonian R, Laird N. 1986. Meta-analysis in clinical trials. Control Clin Trials 7:177-88.
Dixon, J., K. Hovanes, R. Shiang, and M. J. Dixon. 1997. Sequence analysis, identification of evolutionary conserved motifs and expression analysis of murine tcof1 provide further evidence for a potential function for the gene and its human homologue, TCOF1. Hum. Mol. Genet. 6: 727-737.
Dode C, Levilliers J, Dupont J M, et al. 2003. Loss-of-function mutations in FGFR1 cause autosomal dominant Kallmann syndrome. Nat Genet 33:463-5.
Elston R C, Stewart J. 1971. A general model for the genetic analysis of pedigree data. Hum Hered 21:523-42.
Eroshkin, A. & Mushegian, A. 1999. Conserved transactivation domain shared by interferon regulatory factors and Smad morphogens. J. Mol. Med. 77:403-405.
Escalante, C. R., Yie, J., Thanos, D. & Aggarwal, A. K. 1998. Structure of IRF-1 with bound DNA reveals determinants of interferon regulation. Nature 391:103-106.
Fallin M D, Hetmanski J B, Park J, et al. 2003. Family-based analysis of MSX1 haplotypes for association with oral clefts. Genet Epidemiol. 25(2): 168-75.
Field L L, Ray A K, Marazita M L. 1994. Transforming growth factor alpha: a modifying locus for nonsyndromic cleft lip with or without cleft palate? Eur J Hum Genet 2:159-65.
Fitzpatrick, D. R., Denhez, F, Kondaiah, P. & Akhurst R. J. 1990. Differential expression of TGF β isoforms in murine palatogenesis. Development 109, 585-595.
Fraser, F. C. 1955. Thoughts on the etiology of clefts of the palate and lip. Acta Genetica 5: 358-369.
Gorlin, R. J., Sedano, H. O. & Cervenka, J. 1968. Popliteal pterygium syndrome. A syndrome comprising cleft lip-palate, Popliteal and intercrural pterygia, digital and genital anomalies. Pediatrics 41, 503-509.
Horvath S, Xu X, Laird N M. 2001. The family based association test method: strategies for studying general genotype—phenotype associations. Eur J Hum Genet 9:3016.
Houdayer C, Bonaiti-Pellie C, Erguy C, et al. 2001. Possible relationship between the van der Woude syndrome (vWS) locus and nonsyndromic cleft lip with or without cleft palate (NSCL/P). Am J Med Genet 104:86-92.
Jezewski P A, Vieira A R, Nishimura C, et al. 2003. Complete sequencing shows a role for MSX1 in non-syndrommc cleft lip and palate. J Med Genet 40:399-407.
Kaartinen, V. et al. 1995. Abnormal lung development and cleft palate in mice lacking TGF-β3 indicates defects of epithelial-mesenchymal interaction. Nature Genet. 11, 415-421.
Kondo S, Schutte B C, Richardson R J, et al. 2002. Mutations in IRF6 cause Van der Woude and popliteal pterygium syndromes. Nat Genet. 32:285-9.
Laird N M, Horvath S, Xu X. 2000. Implementing a unified approach to family-based tests of association. Genet Epidemiol 19Suppl1:S36-42.
Lees, M. M., Winter, R. M., Malcolm, S., Saal, H. M. & Chitty, L. 1999. Popliteal pterygium syndrome: a clinical study of three families and report of linkage to the Van der Woude syndrome locus on 1q32, J. Med. Genet., 36, 888-892.
Lidral A C, Romitti P A, Basart A M, et al. 1998. Association of MSX1 and TGFB3 with nonsyndromic clefting in humans. Am J Hum Genet 63:557-68.
Lin, R., Heylbroeck C., Genin, P., Pitha, P. M. & Hiscott. J. 1999. Essential role of interferon regulatory factor 3 in direct activation of RANTES chemokine transcription. Mol. Cell Biol. 19, 959-966.
Machin, G. A. 1996. Some causes of genotypic and phenotypic discordance in monozygotic twin pairs. Am. J. Med. Genet. 61, 216-228.
Mamane, Y. et al. 1999. Interferon regulatory factors: the next generation. Gene 237, 1-14.
Marazita M L, Murray J C, Cooper M E, et al. 2003. Meta-analysis of 11 genome scans for cleft lip with or without cleft palate. Am J Hum Genet 73(5):A76.
Marazita M L, Field L L, Cooper M E, et al. 2002. Genome scan for loci involved in cleft lip with or without cleft palate, in Chinese multiplex families. Am J Hum Genet. 71:349-64.
Marazita M L, Field L L, Cooper M E, et al. 2002. Nonsyndromic cleft lip with or without cleft palate in China: assessment of candidate regions. Cleft Palate Craniofac J. 39:149-56.
Marazita M L, Hu D N, Spence M A, Liu Y E, Melnick M. 1992. Cleft lip with or without cleft palate in Shanghai, China: evidence for an autosomal major locus. Am J Hum Genet 51:648-53.
Matzuk, M. M. et al. 1995. Functional analysis of activins during mammalian development, Nature 374, 354-356.
McGrath, J. A. et al. 2001. Hay-Wells syndrome is caused by heterozygous missense mutations in the SAM domain of p63. Hum. Mol. Genet. 10, 22-229.
Mitchell L E, Murray J C, O'Brien S, Christensen K. 2003. Retinoic acid receptor alpha gene variants, multivitamin use, and liver intake as risk factors for oral clefts: a population-based case-control study in Denmark, 1991-1994. Am J Epidemiol 158:69-76.
Moreno L M, Arcos-Burgos M, Marazita M L, et al. 2003. Genetic analysis of candidate loci in non-syndromec cleft lip families from Antioquia-Colombia and Ohio. Am J Med Genet, in press.
Mossey P A and Little J. 2002. Epidemiology of Oral Clefts: An International Perspective. In: Wyszynski D F, ed. Cleft Lip & Palate from Origin to Treatment: Oxford University Press, 127-158.
Murray J C, Daack-Hirsch S, Buetow K H, et al. 1997. Clinical and epidemiologic studies of cleft lip and palate in the Philippines. Cleft Palate Craniofac J. January;34(1):7-10.
Murray, J. C., D. Y. Nishimura, K. H. Buetow, H. H. Ardinger, M. A. Spence, R. S. Sparkes, R. E. Falk, P. M. Falk, R. J. Gardner, E. M. Harkness 1990. Linkage of an autosomal dominant clefting syndrome (Van der Woude) to loci on chromosome 1q. Am. J. Hum. Genet. 46: 486-491.
Nieto, M. A., Patel, K. & Wilkinson, D. G. 1996. In situ hybridization analysis of chick embryos in whole mount and tissue sections. Methods Cell Biol. 51, 219-235.
O'Connell J R, Weeks D E. 1998. PedCheck: a program for identification of genotype incompatibilities in linkage analysis. Am J Hum Genet 63:259-66.
O'Connell J R, Weeks D E. 1995. The VITESSE algorithm for rapid exact multilocus linkage analysis via genotype set-recoding and fuzzy inheritance. Nat Genet 11:402-8.
Page G P, George V, Go R C, Page P Z, Allison D B. 2003. “Are we there yet?”: Deciding when one has demonstrated specific genetic causation in complex diseases and quantitative traits. Am J Hum Genet 73:711-9.
Pritchard J K, Cox N J. 2002. The allelic architecture of human disease genes: common disease-common variant . . . or not? Hum Mol Genet 11:2417-23.
Proetzel, G. et al. 1995. Transforming growth factor-β3 is required for secondary palate fusion. Nature Genet. 11, 409-414.
Rabinowitz D, Laird N. 2000. A unified approach to adjusting association tests for population admixture with arbitrary pedigree structure and arbitrary missing marker information. Hum Hered 50:211-23.
Ray A K, Field L L, Marazita M L. 1993. Nonsyndromic cleft lip with or without cleft palate in West Bengal, India: evidence for an autosomal major locus. Am J Hum Genet 52:1006-11.
Romitti P A, Lidral A C, Munger R G, Daack-Hirsch S, Burns T L, Murray J C. 1999. Candidate genes for nonsyndromic cleft lip and palate and maternal cigarette smoking and alcohol consumption: evaluation of genotype-environment interactions from a population-based case-control study of orofacial clefts. Teratology, 59:39-50.
Sachidanandam, R. et al. 2001. A map of human genome sequence variation containing 1.42 million single nucleotide polymorphisms. Nature 409, 928-933.
Sambrook, J., E. F. Fritsch, and T. Maniatis. 1989. Molecular cloning: A laboratory manual. Cold Spring Harbor Laboratory Press, Cold Spring Harbor, N.Y.
Sander, A., R. Schmelzle, and J. Murray. 1994. Evidence for a microdeletion in 1q32-41 involving the gene responsible for Van der Woude syndrome. Hum. Mol. Genet. 3: 575-578.
Sanford, L. P. et al. 1997. TGFβ2 knockout mice have multiple developmental defects that are non-overlapping with other TGFβ knockout phenotypes. Development 124, 2659-2670.
Satokata I, Maas R. 1994. Msx1 deficient mice exhibit cleft palate and abnormalities of craniofacial and tooth development. Nat Genet 6:348-56.
Schaid D J, Jacobsen S J. 1999. Biased tests of association: comparisons of allele frequencies when departing from Hardy-Weinberg proportions. Am J Epidemiol 149:706-11.
Schliekelman P, Slatkin M. 2002. Multiplex relative risk and estimation of the number of loci underlying an inherited disease. Am J Hum Genet 71:1369-85.
Schultz R E, Cooper M E, Daack-Hirsch S, et al. 2003. A targeted scan of fifteen regions for nonsyndromic cleft lip and palate in Filipino families. Am J Med Genet, in press.
Schutte, B. C. et al. 2000. A preliminary gene map for the Van der Woude syndrome critical region derived from 900 kb of genomic sequence at 1q32-q41 Genome Res. 10, 81-94.
Schutte, B. C., A. M. Basart, Y. Watanabe, J. J. S. Laffin, K. Coppage, B. C. Bjork, S. Daack-Hirsch, S. Patil, M. J. Dixon, and J. C. Murray. 1999. Microdeletions at chromosome bands 1q32-q41 as a cause of Van der Woude syndrome. Am. J. Med. Genet. 84: 145-150.
Shi M, Caprau D, Romitti P, Christensen K, Murray J C. 2003. Genotype frequencies and linkage disequilibrium in the CEPH human diversity panel for variants in folate pathway genes MTHFR, MTHFD, MTRR, RFC1, and GCP2. Birth Defects Res Part A. 67:545-549.
Sobel E, Lange K. 1996. Descent graphs in pedigree analysis: applications to haplotyping, location scores, and marker-sharing statistics. Am J Hum Genet 58:1323-37.
Stanier P F S, Amason A, Bjornsson A, Sveinbjornsdottir E, Williamson R, Moore G. 1993. The localization of a gene causing X-linked cleft palate and ankyloglossia (CPX) in an Icelandic kindred is between DXS326 and DXYS1X. Genomics 17:549-555.
Taniguchi, T., Ogasawara, K., Takaoka, A. & Tanaka, N. 2001. IRF family of transcription factors as regulators of host defense. Annu. Rev. Immunol 19, 623-655.
Terwilliger J D, Off J. 1994. Handbook of Human Genetic Linkage. Johns Hopkins University Press, Baltimore.
van den Boogaard, M. J., Dorland, M., Beemer, F. A. & van Amstel, H. K. 2000. MSX1 mutation is associated with orofacial clefting and tooth agenesis in humans. Nature Genet. 24,342-343.
Van der Woude, A. 1954. Fistula labii inferioris congenita and its association with cleft lip and palate. Am. J. Hum. Genet. 6: 244-256.
Vieira A R, Orioli I M, Castilla E E, Cooper M E, Marazita M L, Murray J C. 2003. MSX1 and TGFB3 contribute to clefting in South America. J Dent Res 82:289-92.
Weinberg C R. 1999. Allowing for missing parents in genetic studies of case-parent triads. Am J Hum Genet 64:1186-93.

1. A method of diagnosing cleft lip and/or palate or other disease states associated with IRF6 dysfunction or dysregulation in a subject, comprising:
obtaining a biological sample from said subject; and
detecting a polymorphism in a IRF6 nucleic acid, wherein the presence of a polymorphism associated with cleft lip and/or palate or other disease state
associated with an IRF6 dysfunction or dysregulation.

2. The method of claim 1, wherein said polymorphism is in exons 2-9 of said IRF6 encoding nucleic acid.

3. The method of claim 1, wherein said nucleic acid is a sequence which encodes a polypeptide sequence comprising amino acids 13-113 of SEQ ID NO:2, wherein said polypeptide comprises at least one polymorphism associated with an IRF6 dysfunction or dysregulation.

4. The method of claim 1, wherein said nucleic acid is a sequence which encodes a polypeptide sequence comprising amino acids 226-394 of SEQ ID NO:2, wherein said polypeptide comprises at least one polymorphism associated with an IRF6 dysfunction or dysregulation.

5. The method of claim 3, wherein said polymorphism is a change from an arginine to cysteine at amino acid residue 84 (Arg84Cys).

6. The method of claim 3, wherein said polymorphism is a change from an arginine to histidine at amino acid residue 84 (Arg84His).

7. The method of claim 5, wherein said polymorphism is characterized by a loss of contact with a DNA binding domain of IRF6.

8. The method of claim 6, wherein said polymorphism is characterized by a loss of contact with a DNA binding domain of IRF6.

9. A method of diagnosing an IRF6 dysfunction or dysregulation in a subject, said method comprising:
obtaining a biological sample from said subject;
detecting the presence of at least one polymorphism as set forth in Table 1 in IRF6 in said sample, wherein the presence of said polymorphism is indicative of said subject having an IRF6 dysfunction or dysregulation.

10. A method of diagnosing a susceptibility or propensity to Van der Woude syndrome, Poplitieal pterygium syndrome, or isolated cleft lip and/or palate in a subject, comprising:
obtaining a biological sample from said subject; and
detecting a polymorphism in an IRF6 encoding nucleic acid present in said sample, wherein the presence of said polymorphism in is indicative of said subject being susceptible to or having a propensity for Van der Woude syndrome, Poplitieal pterygium syndrome, or isolated cleft lip and/or palate.

11. A method of diagnosing a susceptibility to Van der Woude syndrome, Poplitieal pterygium syndrome, or isolated cleft lip and/or palate, comprising
obtaining a biological sample from said subject; and
detecting an alteration in the activity of a polypeptide encoded by an IRF6 gene in a test sample, in comparison with the activity of a polypeptide encoded by IRF6 gene in a control sample, wherein the presence of an alteration in activity of the polypeptide in the test sample is indicative of a susceptibility to Van der Woude syndrome, Poplitieal pterygium syndrome, or isolated cleft lip and/or palate.

12. A method for screening a subject predisposed or susceptible an IRF6-related disorder associated with a genetic polymorphism in the IRF6 gene, said method comprising:
providing a biological sample from the subject; and
testing the sample for the presence of one or more nucleotide mutations from nucleotide positions 1-2171 of SEQ ID NO: 1, wherein, the presence of the mutation indicates that the subject is genetically predisposed to an IRF6-related disorder.

13. A method of diagnosing susceptibility to isolated cleft lip and/or palate in a subject, comprising:
obtaining a biological sample from said subject; and
detecting a polymorphism in a IRF6 gene product, wherein the presence of an amino acid change from a valine to an isoleucine at amino acid residue 274 (Val274Ile) in said gene product is indicative of said subject having a susceptibility to isolated cleft lip and/or palate.

14. A method for diagnosing susceptibility to isolated cleft lip and/or palate in an individual, comprising:
screening for an at-risk genotype in a IRF6 nucleic acid that is more frequently present in an individual susceptible to isolated cleft lip and/or palate (affected), compared to the frequency of its presence in a healthy individual (control), wherein the presence of the at-risk genotype is indicative of a susceptibility to isolated cleft lip and/or palate.

15. The method of claim 14, wherein said at-risk genotype is characterized as a VV genotype at allele 274 of an IRF6 gene product.

16. A method for diagnosing an individual at-risk for isolated cleft lip and/or palate associated with a V allele in an IRF6 gene product comprising:
providing a biological sample from said individual;
determining the genotype of said individual at nucleic acid corresponding to a V274I site of an IRF6 gene product, wherein an individual with two valine encoding alleles at the nucleic acid corresponding to said V274I site possess an at-risk genotype associated with isolated cleft lip and/or palate.

17. The method of claim 16, wherein a valine/valine (VV) encoding genotype is at an increased risk of isolated cleft lip and/or palate than an individual with an isoleucine/isoleucine (I/I) encoding genotype.

18. The method of claim 16, wherein a valine/valine (VV) encoding genotype is at an increased risk of isolated cleft lip and/or palate than an individual with a valine/isoleucine (VI) encoding genotype.

19. A method for screening a subject predisposed or susceptible to Van der Woude syndrome, Poplitieal pterygium syndrome, or isolated cleft lip and/or palate associated with a genetic polymorphism in the IRF6 gene, said method comprising:
providing a biological sample from the subject; and
testing the sample for the presence of one or more nucleotide mutations from nucleotide positions 1-2171 of SEQ ID NO: 1, wherein, the presence of the mutation as shown in Table 1 indicates that the subject is genetically predisposed to Van der Woude syndrome, Poplitieal pterygium syndrome, or isolated cleft lip and/or palate.

20. A method of determining a risk for an IRF6 dysfunction or dysregulation or propensity thereto in a subject, said method comprising:
obtaining a biological sample from said subject;
analyzing the IRF6 nucleic acid in said sample; and
determining the presence of at least one mutation as set forth in Table 1 of IRF6, wherein the presence of said mutation is indicative of a risk for an IRF6 dysfunction or dysregulation or propensity thereto in said subject.

21. The method of claim 20, wherein said IRF6 dysfunction or dysregulation is selected from the group consisting of: Van der Woude syndrome, Poplitieal pterygium syndrome, and isolated cleft lip and/or palate.

22. A method of identifying a polymorphism associated with an IRF6 dysfunction or dysregulation comprising:
isolating a nucleic acid of SEQ ID NO:1 from a plurality of subgroup of subjects, wherein one subgroup has no prevalence for a IRF6 associated disease and at least one or more subgroups which do have a prevalence for a IRF6 associated disease; and
identifying a polymorphism as set forth in Table 1 by comparing the nucleotide sequence of the nucleic acid of the one subgroup having no prevalence for an IRF6 associated disease with the at least one or more subgroups having a prevalence for an IRF6 associated disease.

23. A method of detecting a polymorphism associated with an IRF6 dysfunction or dysregulation comprising:
obtaining a biological sample from said subject; and
detecting a nucleic acid molecule comprising an IRF6 nucleic acid having the nucleotide sequence of SEQ ID NO:1 and comprising at least one polymorphism as shown in Table 1.

24. The method of claim 23, wherein said IRF6 dysfunction or dysregulation is selected from the group consisting of: Van der Woude syndrome, Poplitieal pterygium syndrome, and isolated cleft lip and/or palate.

25. A method of identifying an agent which modulates activity of a polypeptide encoded by an IRF6 nucleic acid, wherein said polypeptide comprises an amino acid sequence as depicted in SEQ ID NO:2 and comprises at least one polymorphism as shown in Table 1 comprising:
contacting said polypeptide variant with an agent to be tested;
assessing the level of activity of the IRF6 variant polypeptide; and
comparing the level of activity with a level of activity of said variant polypeptide in the absence of the agent, wherein if the level of activity of the polypeptide in the presence of the agent differs, by an amount that is statistically significant from the level in the absence of the agent, then the agent is an agent that modulates activity of a variant IRF6 polypeptide.

26. A method of identifying an agent which alters expression of a variant IRF6 nucleic acid, comprising:
contacting a variant nucleic acid comprising SEQ ID NO:1 and comprising at least one polymorphism at shown in Table 1 with an agent to be tested;
assessing the level of expression of said variant nucleic acid; and
comparing the level of expression with a level of expression of said variant nucleic acid in the absence of the agent, wherein if the level of expression of the nucleotide in the presence of the agent differs, by an amount that is statistically significant, from the expression in the absence of the agent, then the agent is an agent that alters expression of a variant IRF6 nucleic acid.

27. A method of identifying and obtaining an inhibitor of the activity of a polypeptide, or a derivative, or fragment thereof comprising the amino acid sequence of SEQ ID NO:2 which comprises at least one polymorphism as shown in Table 1, comprising:
contacting said polypeptide, or derivative, or fragment thereof with a test agent for inhibiting activity, in the presence of compounds that provide a detectable symbol in response to test agent activity; and
detecting the presence or absence of a signal or increase or decrease of a signal generated from inhibiting activity, wherein the absence or decrease of the signal is indicative inhibiting activity of said polypeptide.

28. The method of claim 1, wherein said detecting comprising contacting said sample with a solid support under conditions allowing interaction of said IRF6 nucleic acid to said immobilized targets on said solid support.

29. The method of claim 28, wherein a binding of a variant IRF6 nucleic acid to said immobilized targets on said solid support is indicative of the presence, absence, or prevalence of an IRF6 dysfunction or dysregulation.

30. The method of claim 1, wherein said detecting comprising contacting said sample with a solid support under conditions allowing interaction of an IRF6 nucleic acid comprising the nucleotide sequence of SEQ ID NO:1 and comprising at least one polymorphism as shown in Table 1.

31. The method of claim 1, wherein said detecting comprising contacting said sample with a solid support under conditions allowing interaction of a vector, wherein said vector comprises an isolated nucleic acid comprising SEQ ID NO:1 and comprising at least one polymorphism as shown in Table 1, operatively linked to a regulatory sequence to said immobilized targets on said solid support.

32. The method of claim 1, wherein said detecting comprising contacting said sample with a solid support under conditions allowing interaction of a recombinant host cell, present in said sample.

33. The method of claim 1, wherein said detecting comprising contacting said sample with a solid support under conditions allowing interaction of an isolated polypeptide encoded by an IRF6 nucleic acid present in said sample, said polypeptide comprising:
(a) the amino acid sequence as depicted in SEQ ID NO:2 which comprises at least one polymorphism as shown in Table 1;
(b) the amino acid sequence as depicted in SEQ ID NO:2 which comprises at least two polymorphisms as shown in Table 1;
(c) an amino acid sequence which is greater than about 90% identical to an amino sequence of SEQ ID NO:2 and comprising at least one polymorphism as shown in Table 1; and
(d) an amino acid sequence which is greater than about 95% identical to an amino acid sequence of SEQ ID NO:2 and comprising at least one polymorphism as shown in Table 1.

34. The method of claim 1, wherein said detecting comprising contacting said sample with a solid support under conditions allowing interaction of an antibody, or a fragment thereof, that specifically binds to the polypeptide encoded by an IRF6 nucleic acid comprising:
(a) the amino acid sequence as depicted in SEQ ID NO:2 which comprises at least one polymorphism as shown in Table 1;
(b) the amino acid sequence as depicted in SEQ ID NO:2 which comprises at least two polymorphisms as shown in Table 1;
(c) an amino acid sequence which is greater than about 90% identical to an amino sequence of SEQ ID NO:2 and comprising at least one polymorphism as shown in Table 1; and
(d) an amino acid sequence which is greater than about 95% identical to an amino acid sequence of SEQ ID NO:2 and comprising at least one polymorphism as shown in Table 1.

35. The method of claim 1, wherein said detecting comprising contacting said sample with a solid support under conditions allowing interaction of a nucleic acid molecule comprising exons 2-9 of IRF6 having a nucleotide sequence comprising SEQ ID NO:1 and comprising at least one polymorphism associated with Van der Woude syndrome, Poplitieal syndrome, and isolated cleft lip and/or palate in any one of said exons.

36. The method of claim 1, wherein said detecting comprising contacting said sample with a solid support under conditions allowing interaction of an IRF6 nucleic acid having a nucleotide sequence depicted in SEQ ID NO:1, wherein said nucleotide sequence encodes a polypeptide sequence comprising amino acids 13-113 of SEQ ID NO:2, wherein said polypeptide comprises at least one polymorphism as shown in Table 1.

37. The method of claim 1, wherein said detecting comprising contacting said sample with a solid support under conditions allowing interaction of an IRF6 nucleic acid having a nucleotide sequence depicted in SEQ ID NO:1, wherein said nucleotide sequence encodes a polypeptide sequence comprising amino acids 226-394 of SEQ ID NO:2, wherein said polypeptide comprises at least one polymorphism as shown in Table 1.

38. A diagnostic composition for detecting the predisposition to an IRF6 dysfunction or dysregulation, the composition comprising at a nucleic acid comprising SEQ ID NO:1 and comprising at least one polymorphism, and a carrier.

39. The diagnostic composition of claim 38 comprising a nucleic acid sequence comprising SEQ ID NO:1 and comprising at least one polymorphism as shown in Table 1, and a carrier.

40. A transgenic non-human animal comprising
(a) a nucleic acid molecule comprising an IRF6 nucleic acid having the nucleotide sequence having SEQ ID NO:1 and comprising at least one polymorphism as shown in Table 1;
(b) a nucleic acid molecule comprising an IRF6 gene, the nucleotide sequence of SEQ ID NO:1 encoding a polypeptide comprising an amino acid sequence as depicted in SEQ ID NO:2 and comprises at least one polymorphism associated as shown in Table 1.

41. An isolated nucleic acid molecule comprising:
(a) a nucleic acid molecule comprising an IRF6 nucleic acid having the nucleotide sequence of SEQ ID NO:1 and comprising at least one polymorphism as shown in Table 1;
(b) a nucleic acid molecule comprising an IRF6 nucleic acid having the nucleotide sequence of SEQ ID NO:1 and comprising at least two polymorphism as shown in Table 1; and
(c) a nucleic acid molecule comprising an IRF6 gene, the nucleotide sequence of SEQ ID NO:1 encoding a polypeptide comprising an amino acid sequence as depicted in SEQ ID NO:2 and comprises at least one polymorphism associated as shown in Table 1.

42. A vector comprising an isolated nucleic acid molecule comprising SEQ ID NO:1 and comprising at least one polymorphism shown in Table 1, operatively linked to a regulatory sequence.

43. A recombinant host cell comprising the vector of claim 42.

44. An isolated nucleic molecule comprising an IRF6 nucleic acid having a nucleotide sequence depicted in SEQ ID NO:1, wherein said nucleotide sequence encodes a polypeptide sequence comprising amino acids 13-113 of SEQ ID NO:2, wherein said polypeptide comprises at least one polymorphism as shown in Table 1.

45. An isolated nucleic molecule comprising an IRF6 nucleic acid having a nucleotide sequence depicted in SEQ ID NO:1, wherein said nucleotide sequence encodes a polypeptide sequence comprising amino acids 226-394 of SEQ ID NO:2, wherein said polypeptide comprises at least one polymorphism as shown in Table 1.

46. The isolated nucleic acid molecule of claim 44, said polymorphism is a change from arginine to a cysteine at amino acid residue 84 (Arg84Cys).

47. The isolated nucleic acid molecule of claim 46, said polymorphism is a change from a cytosine to a thymine at nucleotide position 250 (C250T).

48. The isolated nucleic acid molecule of claim 44 said polymorphism is a change from an arginine to a histidine at amino acid residue 84 (Arg84His).

49. The isolated nucleic acid molecule of claim 48, said polymorphism is a change from a guanine to an adenine at nucleotide position 251 (G251A).

50. An isolated nucleic acid molecule comprising: exons 2-9 of an IRF6 nucleic acid comprising SEQ ID NO:1, wherein at least one has at least one polymorphism as shown in Table 1.

51. An isolated polypeptide encoded by an IRF6 nucleic acid comprising:
(a) the amino acid sequence as depicted in SEQ ID NO:2 which comprises at least one polymorphism as shown in Table 1;
(b) the amino acid sequence as depicted in SEQ ID NO:2 which comprises at least two polymorphisms as shown in Table 1;
(c) an amino acid sequence which is greater than about 90% identical to an amino sequence of SEQ ID NO:2 which comprises at least one polymorphism as shown in Table 1; and
(d) an amino acid sequence which is greater than about 95% identical to an amino acid sequence of SEQ ID NO:2 which comprises at least one polymorphism as shown in Table 1.

52. A fusion protein comprising the polypeptide of claim 51.

53. An antibody, or an antibody fragment thereof, that specifically binds to the polypeptide of claim 51.

54. An antibody, or antibody fragment thereof that specifically binds to the polypeptide of claim 44.

55. An antibody or antibody fragment thereof that specifically binds to the polypeptide of claim 45.

56. An isolated nucleic acid comprising at least 20-50 contiguous nucleotides, wherein said nucleic acid comprises the nucleotide sequence of SEQ ID NO:1 and comprises at least one polymorphism as shown in Table 1.

57. The nucleic acid molecule of claim 41, wherein the nucleic acid is DNA.

58. The nucleic acid molecule of claim 41, wherein the nucleic acid is RNA.

59. The nucleic acid of claim 56, wherein the nucleic acid is DNA.

60. The nucleic acid of claim 56, wherein the nucleic acid is RNA.

61. An isolated polypeptide encoded by an IRF6 nucleic acid comprising: the amino acid sequence as depicted in SEQ ID NO:2, wherein said polypeptide sequence comprises a polymorphism of valine to isoleucine at amino acid residue 274 (V274Ile).

62. A pharmaceutical composition comprising the polymorphic nucleic acid of claim 41 and a physiologically acceptable carrier.

63. A pharmaceutical composition comprising the vector of claim 42, and a physiologically acceptable carrier.

64. A pharmaceutical composition comprising the polypeptide of claim 51, and a physiologically acceptable carrier.

65. A pharmaceutical composition comprising the fusion protein of claim 52, and a physiologically acceptable carrier.

66. A pharmaceutical composition comprising the antibody of claim 53 and a physiologically acceptable carrier.


SEQUENCE LISTING (4 sequences)

SEQ ID NO: 1 - DNA, 2171 bp, Homo sapiens
  5′UTR (215)..(215)
  variation (216)..(216): Frame shift to encode a MET.  A to T (exon 2).
gagctcggcgcacctgggctgggcaggtaagggctggtgcgggacggggagaggaacctgcagtccctacttgggtagagccaggcgccccttggctaagacgtcgaggagcgtggtagcgacgggtgatcttcgctgcggacttggttcggagggacgtccgcttctggtggacagattgagcaaagaatctttgagcggtcaagggaaagacaagccgactcttcagatccctgtggacacactgcctgctcttccatatcatggccctccacccccgcagagtccggctaaagccctggctggtggcccaggtggatagtggcctctaccctgggctcatctggctacacagggactctaaacgcttccagattccctggaaacatgccacccggcatagccctcaacaagaagaggaaaataccatttttaaggcctgggctgtagagacagggaagtaccaggaaggggtggatgaccctgacccagctaaatggaaggcccagctgcgctgtgctctcaataagagcagagaattcaacctgatgtatgatggcaccaaggaggtgcccatgaacccagtgaagatatatcaagtgtgtgacatccctcagccccagggctcgatcattaacccaggatccacagggtctgctccctgggatgagaaggataatgatgtggatgaagaagatgaggaagatgagctggatcagtcgcagcaccatgttcccatccaggacaccttccccttcctgaacatcaatggttctcccatggcgccagccagtgtgggcaattgcagtgtgggcaactgcagcccggaggcagtgtggcccaaaactgaacccctggagatggaagtaccccaggcacctatacagcccttctatagctctccagaactgtggatcagctctctcccaatgactgacctggacatcaagtttcagtaccgtgggaaggagtacgggcagaccatgaccgtgagcaaccctcagggctgccgactcttctatggggacctgggtcccatgcctgaccaggaggagctctttggtcccgtcagcctggagcaggtcaaattcccaggtcctgagcatattaccaatgagaagcagaagctgttcactagcaagctgctggacgtcatggacagaggactgatcctggaggtcagcggtcatgccatttatgccatcaggctgtgccagtgcaaggtgtactggtctgggccatgtgccccatcacttgttgctcccaacctgattgagagacaaaagaaggtcaagctattttgtctggaaacattccttagcgatctcattgcccaccagaaaggacagatagagaagcagccaccgtttgagatctacttatgctttggggaagaatggccagatgggaaaccattggaaaggaaactcatcttggttcaggtcattccagtagtggctcggatgatctacgagatgttttctggtgatttcacacgatcctttgatagtggcagtgtccgcctgcagatctcaaccccagacatcaaggataacatcgttgctcagctgaagcagctgtaccgcatccttcaaacccaggagagctggcagcccatgcagcccacccccagcatgcaactgccccctgccctgcctccccagtaattgtgaatgccatcttcttccttctcttttttataatattgtacatatggatttttttattgtttagatttaaccagcttttaaatctctcttttctctaacagtgttagaagtttgtgattctccaaatatgcctagatttaaagctgatttaatttatggaaaaatcacccttcagactttgcttttctttttcaaatctcctaatggtagtatgatatagcatagtagaaggagatttggcctgggagtttggacaccaaagttctagctgcagctttgcttccaatgtgaccttgaacaagtcctttaacctctgggcttcagatttattgcttataaagtgaagagattggagtagtgcctgaaattgcatccagctttagaacggactcaatgaccttcttctacttgtacaaggctaaactgcctggaacagaatccttctgcattgttcttgtaccacatttttccttggttttgttaaagtttcctcaagcacta

SEQ ID NO: 2 - protein, 467 aa, Homo sapiens
MALHPRRVRLKPWLVAQVDSGLYPGLIWLHRDSKRFQIPWKHATRHSPQQEEENTIFKAWAVETGKYQEGVDDPDPAKWKAQLRCALNKSREFNLMYDGTKEVPMNPVKIYQVCDIPQPQGSIINPGSTGSAPWDEKDNDVDEEDEEDELDQSQHHVPIQDTFPFLNINGSPMAPASVGNCSVGNCSPEAVWPKTEPLEMEVPQAPIQPFYSSPELWISSLPMTDLDIKFQYRGKEYGQTMTVSNPQGCRLFYGDLGPMPDQEELFGPVSLEQVKFPGPEHITNEKQKLFTSKLLDVMDRGLILEVSGHAIYAIRLCQCKVYWSGPCAPSLVAPNLIERQKKVKLFCLETFLSDLIAHQKGQIEKQPPFEIYLCFGEEWPDGKPLERKLILVQVIPVVARMIYEMFSGDFTRSFDSGSVRLQISTPDIKDNIVAQLKQLYRILQTQESWQPMQPTPSMQLPPALPPQ

SEQ ID NO: 3 - protein, 467 aa, Homo sapiens; identical to SEQ ID NO: 2
  MISC_FEATURE: 5′ UTR to Met

SEQ ID NO: 4 - DNA, 19 bp, Homo sapiens
  misc_feature: 870del(CACTAGCAAGCTGCTGGAC)lns (A)
cactagcaagctgctggac